(19) United States
(12) Patent Application Publication
(10) Pub. No.: US 2007/0065443 A1
(43) Pub. Date: Mar. 22, 2007

(54) Fructoseamine 3 kinase and the formation of collagen and elastin

(76) Inventors: Annette Tobia, Wyndmoor, PA (US); Francis Kappler, Philadelphia, PA (US); Michael L. Schwartz, Palmyra, NJ (US)

Correspondence Address: Kathryn Doyle, Ph.D., J.D.;Drinker Biddle & Reath, One Logan Square, 18th & Cherry Streets, Philadelphia, PA, 19103, US

(21) Appl. No.: 11/499,497

(22) Filed: Aug. 4, 2006

Related U.S. Application Data
(63) Continuation of application No. PCT/US05/05082, filed on Feb. 17, 2005.
(60) Provisional application No. 60/545,035, filed on Feb. 17, 2004.
(60) Provisional application No. 60/545,036, filed on Feb. 17, 2004.

(51) Int. Cl. A61K 38/55; A61K 39/395; A61K 31/275; A61K 31/22; A61K 31/18; A61K 31/16

(52) U.S. Cl. 424/146.1; 514/18; 514/602; 514/616; 514/702; 514/566; 514/521; 514/546

(57) ABSTRACT

The invention relates to the discovery that levels of collagen and elastin can be modulated by changing the flux through the Amadori Pathway and that copper containing compounds and complexes inhibit the enzyme fructoseamine-3-kinase.

### BACKGROUND OF THE INVENTION

Tissue flexibility and extensibility have been essential requirements in the evolution of multicellular organisms. Collagen and elastic fibers are the major components of the insoluble extracellular matrix (ECM) that endows connective tissues with tensile strength and resilience, permitting long-range deformability and passive recoil without energy input. These properties are critical to the function of arteries, which undergo repeated cycles of extension and recoil, and to the lungs, skin and all other dynamic connective tissues.

Collagens are insoluble, extracellular glycoproteins that are found in all animals and are the most abundant proteins in the human body. They are essential structural components of all connective tissues, such as cartilage, bone, tendons, ligaments, fascia and skin. Collagens are centrally involved in the formation of fibrillar and microfibrillar networks of the extracellular matrix, basement membranes as well as other structures of the extracellular matrix (Gelse, K. et al., 2003, Adv Drug Deliv Rev 55:1531-46).

Collagens are the main proteins responsible for the structural integrity of vertebrates and many other multicellular organisms. In tissues like skin, tendons, bone and cartilage, collagen fibrils provide resistance to tensile stress. Depending on the tissue, fibrils are arranged with different suprafibrillar architectures and with diameters up to 500 nm. Small diameter fibrils are found in cartilage and also in cornea, where in the latter the highly ordered arrangement of fibrils within orthogonal lamellae is essential for optical transparency. All fibrillar collagens are synthesized and secreted into the extracellular matrix in the form of soluble precursors called procollagens. Fibril-forming collagens (type I, II, III, V and XI) account for only 5 of more than 20 different genetic types of collagen in humans. All collagens are modular proteins consisting of three polypeptide chains with at least one stretch of triple helix.

Of the collagens found in humans, types I-IV are the most abundant. Type I is the chief component of tendons, ligaments, and bone. Type II collagen represents more than 50% of the protein in cartilage. It is also used to build the notochord of vertebrate embryos. Type III strengthens the walls of hollow structures like arteries, the intestine, and the uterus. Type IV forms the basal lamina of epithelia which is often called the basement membrane. A meshwork of type IV collagen provides the filter for blood capillaries and kidney glomeruli. The other 15 types are probably equally important but they are much less abundant.

The basic collagen unit is a polypeptide consisting of the repeating sequence (glycine (Gly)-X-Y)ₙ, where X is often proline (Pro) and Y is often hydroxyproline (proline to which an —OH group is added after synthesis of the polypeptide). To form the secondary and tertiary structure, the molecule twists into an elongated, left-handed helix. When synthesized, the N- and C-terminii of the polypeptide have globular domains, which keep the molecule soluble. As they pass through the endoplasmic reticulum (ER) and Golgi apparatus, the molecules are glycosylated, and hydroxyl groups are added to produce the “Y” amino acid. Interchain disulfide bonds covalently link three chains and the three molecules twist together to form a triple helix.When the triple helix is secreted from the cell, usually by a fibroblast, the globular ends are cleaved off. The resulting linear, insoluble molecules assemble into collagen fibers. They assemble in a staggered pattern that gives rise to the striations seen in electron micrographs. Type IV collagens are an exception because they form a meshwork rather than striated fibers.

In some collagens (e.g., type II), the three molecules are identical (the product of a single gene). In other collagens (e.g., type I), two polypeptides of one kind (gene product) assemble with a second, quite similar, polypeptide, that is the product of a second gene.

In skin, the dermis layer is composed largely of collagen bundles running horizontally, which are buried in a jelly-like material called the ground substance. Collagen is the main component of the dermis constituting 75% of the dry weight. More than 70% is type I collagen and 15% is type III collagen. The size and arrangement of the collagen fibers distinguishes two dermal regions in adult skin. The papillary dermis, which interdigitates with the epidermis is a well-vascularized area composed mainly of type III collagen, also known as reticulin. The collagen fibers are narrow, short, loosely interwoven, randomly oriented and embedded within the ground substance. The reticular dermis is composed mainly of type I collagen, with collagen fibers that are wider and tightly packed together in large, broad and wavy bundles. These bundles are loosely interwoven, arranged parallel with the skin surface and also embedded in ground substance (Lavker et al., 1987, J. Invest. Dermatol. 88:44-51).

The natural aging process decreases collagen synthesis and increases the expression of matrix metalloproteinases, whereas photo aging results in an increase of collagen synthesis and a corresponding greater amount of matrix. (Chung et al., 2001, J. Invest. Dermatol. 117:1218-24). It has also been discussed that type I collagen synthesis diminishes with age in eyelid skin (DeBacker et al., 1998, Ophthal. Plast. Reconstr. Surg. 14:13-16).

Collectively, the aging processes, whether intrinsic or extrinsic, have both quantitative and qualitative effects on collagen and elastic fibers in the skin (El-Domyati et al., 2002, Exp. Dermatol. 11:398-405). Naturally aged, sun-protected skin and photo aged skin share important molecular features including connective tissue damage, elevated matrix metalloproteinase levels, and reduced collagen production. (Varani et al., 2000, J. Invest. Dermatol. 114:480-6).

Although type IV collagen is a basement membrane component and declines with aging, the total thickness of this membrane increases, which suggests a reduction in tissue turnover (Vazquez et al., 1996, Maturitas 25:209-15). Superficial dermabrasion clinically improves photo aged skin, and this improvement correlates strongly with increased collagen I gene expression (Nelson et al., 1994, Arch. Dermatol. 130:1136-42).

Aging involves dermal changes such as damage to elastic and collagen fibers thus giving rise to thickened, tangled, and degraded non-functional fibers. Cross-linking of collagen is influenced by many factors and the crosslinking pattern may, therefore, reflect the structural status of the collagen fibrils. Collagen intermolecular crosslinks are stable and essential for stability and tensile strength. With age, skin stiffness increases, concomitantly with an increase in collagen crosslinks. Divalent crosslinks are converted into mature trivalent crosslinks of, e.g. histidinohydroxylysinonorleucine. Two mechanisms are involved: an enzyme-controlled process of maturation and a non-enzymatic glycosylation, the Maillard reaction, leading to crosslinks in proteins such as between arginine and lysine in collagen. Such may be seen with age and in diabetes mellitus. However, auto fluorescence studies have shown that UVR reduces collagen crosslinks.

The changes related to chronic UVR exposure might be due to the loss of collagen, which is compensated for either by the elastotic material that is compact and uniform or by a mixture of water and ground substance (de Rigal et al., 1989, J. Invest. Dermatol. 93:621-5). Changes in collagen composition might also play a role. In accordance it has been shown that the proportion of collagen type III is increased in photo-damaged skin (Plastow et al., 1987, J. Invest. Dermatol. 88:145-8).

Abnormal production of collagen as well as mutations in the collagen gene can result in various diseases. Collagen type VI appears to be related to a very common eye problem known as age-related macular disease (AND). AMD is a disease that affects the macula, and blurs the sharp, central vision needed for activities such as reading, sewing, and driving. Little is known about the pathogenesis of this condition, but deposits in Bruch's membrane and immediately beneath the retinal pigment epithelium are frequent findings associated with this disease. Two types of assembly are present: one exhibiting transverse double bands of protein density that are 30 nm apart and repeat axially every approximately 100 nm; the other with transverse double bands of protein density, 30 nm apart and repeating axially every approximately 50 nm. (Knupp et al., 2002, J. Struct. Biol. 137:31-40). AMD shares many clinical and pathological features with Sorsby's fundus dystrophy (SFD), an autosomal dominant disease, that is associated with mutations in the tissue inhibitor of metalloproteinase-3 (TIMP-3) gene.

Osteoarthritis is a chronic disease characterized by progressive destruction of articular cartilage and subchondral bone and synovial reaction. Osteoarthritis and intervertebral disc disease are the most common musculoskeletal disorders. Although they are associated with a number of risk factors, recent results suggest that genetic factors may play a major role in their pathogenesis. Both hyaline cartilage and intervertebral disc contain relatively few cells but an abundant extracellular matrix. Since osteoarthritis and disc disease are characterized by degeneration of hyaline cartilage and intervertebral disc, these genetic factors may include genes coding for connective tissue proteins such as collagens.

Cartilage collagens (collagens II, IX and XI) are found in hyaline cartilage and intervertebral disc. Collagen II is the most abundant protein in hyaline cartilage, with the interior structure of an intervertebral disc, the nucleus pulposus, containing 20% of its dry weight as collagen II. Collagens IX and XI are quantitatively minor components in hyaline cartilage and intervertebral disc. In addition to the nucleus pulposus, collagen IX is also found in the outer layer of the disc, the annulus fibrosis. Collagen II, together with collagens IX and XI, forms a strong framework of fibrils with a tensile strength comparable to that of steel. Collagens II and XI belong to the group of fibril-forming collagens. Mutations in collagen II have relatively severe phenotypes and can result in a spectrum of diseases varying from chondrodysplasias to osteoarthritis. This finding most likely reflects the importance of collagen H in the development and mechanical support of the tissue (Ala-Kokko et al., 1990, Proc. Natl. Acad. Sci. U.S.A. 87:6565-8). (Kotaniemi et al., 2003, Clin. Exp. Rheumatol. 21:95-8).

The myocardial collagen matrix consists of a network of fibrillar collagen which is intimately connected to the myocyte. Fibrillar collagen types I and III are the major components of the myocardial collagen matrix. They reside in parallel with myocytes, and have a wavy, taut or coiled appearance. Collagen type I has been found to represent nearly 80% of the total collagen protein, while type III collagen is present in lower proportions (approximately 11%). Cardiac fibroblasts are the cellular source of fibrillar collagen, with cardiac myocytes expressing only mRNA for type IV collagen. Collagen types I and III exhibit a high tensile strength which plays an important role in the behavior of the ventricle during the cardiac cycle. The collagen concentration and the intermolecular crosslinking of collagen increase with age. Measurements of collagen content in myocardial tissue suggest that it is the type I collagen fibers that increase in number and thickness in the aged. At the same time, electron microscopic observations have shown an increase in the number of collagen fibrils with a large diameter in the aging heart. The mechanism responsible for the myocardial fibrosis in the senescent myocardium is unclear. The collagen deposition in the myocardium could be due to the regulation of collagen biosynthesis at pre-translational levels. It is possible that the regulatory elements involved in this process are growth factors such as TGF-beta 1 and hormones and neurotransmitters. Details of the regulatory mechanisms that may come into play during aging may be elucidated by further investigations.

The accumulation of collagen within the myocardium increases muscle stiffness. Myocardial function is affected by this process; this is usually reflected by incomplete relaxation during early diastolic filling, and presumably account for the decrease in early left ventricular diastolic compliance (de Souza, 2002, Biogerontology 3:325-35). Fibrous tissue accumulation is an integral feature of the adverse structural remodeling of cardiac tissue seen with hypertensive heart disease. (Lopez et al., 2001, Circulation 104:286-91).

Aging and diabetes mellitus (DM) both affect the structure and function of the myocardium, resulting in increased collagen in the heart and reduced cardiac function. As part of this process, hyperglycemia is a stimulus for the production of advanced glycation end products (AGEs), which covalently modify proteins and impair cell function (Liu et al., 2003, Am. J. Physiol. Heart. Circ. Physiol. 285:2587-91).

Collagen levels are altered as a result of inflammatory processes. In order to investigate the properties of collagen in chronically inflamed tissue, collagen from the ear skin of mice with chronic contact dermatitis was isolated and examined for its biochemical characteristics that regulate the secretion of matrix metalloproteinase 2 and other collagen-degrading enzymes from endothelial cells and fibroblasts. Collagen in skin with chronic contact dermatitis is comprised of 60% type I collagen and 40% type III collagen, of which the latter is higher than the content in control skin. Collagen-degrading activity secreted from fibroblasts was also upregulated when cells were in contact with collagen of chronically inflamed skin. These results suggest that the collagen in chronically inflamed tissue has altered biochemical characteristics and functions, which may affect the pathogenesis of chronic skin disease (Hirota et al., 2003, J. Invest. Dermatol. 121:1317-25).

Crosslinking of collagen type I and type IV by UV irradiation was also observed. Amino acid analyses revealed that Tyr residues in both collagen types were decreased by irradiation, and losses of His and Met residues were also observed in collagen type IV. These losses of collagen type IV may be due to the degradation of Trp, which is present in collagen type IV and decreased dramatically during UV irradiation (Kato et al., 1995, Photochem. Photobiol. 61:367-72).

Another disease related to collagen abnormality is endomyocardial fibrosis. This is a distinct form of heart disease leading to restrictive ventricular filling and cardiac failure. The disease is characterized by a marked thickening of the endocardium due to the deposition of dense fibrous tissue composed of wavy bundles of collagen. (Radhakumary et al., 2001, Indian Heart J. 53:486-9).

Pulmonary fibrosis is a disorder causing a high mortality rate for which therapeutic options are limited. Therefore, the effect of halofuginone, a novel inhibitor of collagen type I synthesis, on bleomycin-induced pulmonary fibrosis was studied in rats. Halofuginone is a potent in vivo inhibitor of bleomycin-induced pulmonary fibrosis, and that it may potentially be used as a novel therapeutic agent for the treatment of this dysfunction (Nagler et al., 1996, Am. J. Respir. Crit. Care Med. 154:1082-6). Another disease, adult respiratory distress syndrome (ARDS), is an inflammation of the lungs which become stiff and fibrous and cannot exchange oxygen. (Deheinzelin et al., 1997, Chest 112:1184-8).

The development of high myopia is associated with reduced scleral collagen accumulation, scleral thinning, and loss of scleral tissue, in both humans and animal models. Reduced collagen fibril diameter is also observed in the sclera of eyes with high myopia. The majority of the collagens investigated were found to be expressed in the sclera, with 11 subtypes being identified. Collagen type I mRNA expression was reduced in the sclera of myopic eyes, however, collagen type III and type V expression was unchanged relative to control, resulting in a net increase in the ratio of expression of collagen type III/type I and collagen type V/type I. These results show that reduced scleral collagen accumulation in myopic eyes is a result of both decreased collagen synthesis and accelerated collagen degradation. Furthermore, changes in collagen synthesis are driven by reduced type I collagen production. Short term increases in the ratio of newly synthesized collagen type III/type I and type V/type I are likely to be important in the increasing frequency of small diameter scleral collagen fibrils observed in high myopia and may be important in the subsequent development of posterior staphyloma in humans with pathological myopia (Gentle et al., 2003, J. Biol. Chem. 278:16587-94). (Sagara et al., 1999, Invest. Ophthalmol. Vis. Sci. 40:2568-76).

Excessive deposition of collagen has been implied to be responsible for abnormal stiffness and altered cardiac function during hypertrophy and heart failure. Data showed that during the chronic phase of hypertrophy in spontaneous hypertensive rats (SHR) there is a gradual reduction in the type I to III ratio, primarily due to a lack of increase in type III collagen during chronic phase of hypertrophy. This suggests that quality of collagen is an important factor in determining the degree of cardiac stiffness [Yang et al., 1997, Cardiovasc. Res. 36:236-45).

Osteogenesis imperfecta (OI), commonly known as “brittle bone disease”, is a dominant autosomal disorder characterized by bone fragility and abnormalities of connective tissue. Biochemical and molecular genetic studies have shown that the vast majority of affected individuals have mutations in either the COL1A1 or COL1A2 genes that encode the chains of type I procollagen. OI is associated with a wide spectrum of phenotypes varying from mild to severe and lethal conditions. The mild forms are usually caused by mutations which inactivate one allele of COL1A1 gene and result in a reduced amount of normal type I collagen, while the severe and lethal forms result from dominant negative mutations in COL1A1 or COL1A2 which produce structural defects in the collagen molecule. The most common mutations are substitutions of glycine residues, which are crucial to formation and function of the collagen triple helix, by larger amino acids. Although type I collagen is the major structural protein of bone and skin, the mutations in type I collagen genes cause a bone disease. Some reports showed that the mutant collagen can be expressed differently in bone and in skin. Since most OI mutations are dominant negative, the gene therapy requires a fundamentally different approach from that used for genetic-recessive disorders. Antisense therapy, by reducing the expression of mutant genes, may be able to change a structural mutation into a null mutation, and thus convert severe forms of the disease into mild OI type I (Gajko-Galicka, 2002, Acta. Biochim. Pol. 49:433-41). (Cabral et al., 2003, J. Biol. Chem. 278:10006-12). (Nuytinck et al., 1997, Eur. J. Hum. Genet. 5:161-7).

Yet another disease that can be brought about by defects in collagen is Heterotopic Ossification (HO).It can occur as a consequence of several diseases and of various forms of trauma. In HO, chondrogenic cells play a central role to produce the HO phenotype due to alterations in collagen and TGF-beta 1 mRNA expression (Bosse et al., 1994, Pathologe 15:216-25).

Scleroderma or systemic sclerosis (SSc), is a chronic, autoimmune disease of the connective tissue generally classified as one of the rheumatic diseases. It is a disease in which the symptoms may either be visible, as when the skin is affected, or invisible, as when only internal organs are involved. It brings about thickening, hardening, or tightening of the skin, blood vessels and internal organs. Scleroderma is a highly-individualized disease that can be manifested from mild symptoms to life-threatening. The disease is characterized by excessive collagen synthesis by fibroblasts and by vascular hyper reactivity and obliteration phenomena. Excessive collagen production is the consequence of abnormal interactions between endothelial cells, fibroblasts and mononuclear cells. Immunological abnormalities are present very early in the development of SSc. Cytokines from mononuclear cells, particularly macrophages and T lymphocytes, play a prominent role in fibroblast activation and collagen synthesis. Lymphocytic infiltrates in the skin and in the lung are preferentially composed of CD8+ T lymphocytes that produce interleukin 4 (IL-4). The effects of IL4 combined with transforming growth factor B (TGF-B) and connective tissue growth factor (CTGF) stimulate collagen synthesis by fibroblasts. T lymphocytes also produce gamma interferon (INF-gamma), an effective inhibitor of collagen synthesis by fibroblasts. However, the inhibitory effect of INF-gamma on collagen synthesis is diminished in SSc patients. Numerous autoantibodies are also present in the serum of SSc patients (Mouthon et al., 2002, Ann. Med. Intene. 153:167-78).

Upregulation of collagen gene expression in SSc fibroblasts appears to be a critical event in the development of tissue fibrosis. The coordinate transcriptional activation of a number of extracellular matrix genes suggests a fundamental alteration in the regulatory control of gene expression in SSc fibroblasts. (Jimenez et al., 1996, Rheum. Dis. Clin. North Am. 22:647-74).

Scleroderma is characterized by fibrosis involving the skin and various internal organs. Type I collagen (Col I) is the most abundant extracellular matrix protein deposited in cutaneous involvement (Allanore et al., 2003, J. Rheumatol. 30:68-73). The synthesis of the alpha1 and alpha2 collagen polypeptides that comprise type I collagen is highly transcriptionally regulated by different cytokines. Excessive synthesis and deposition of collagen in the dermal region causes thick and hard skin, a clinical manifestation of scleroderma (Ghosh, 2002, Exp. Biol. Med. 227:301-14). Scleroderma also includes Morphea Scleroderma, or localized scleroderma.

Chronic graft-versus-host disease (cGvHD) and scleroderma share clinical characteristics, including skin and internal organ fibrosis. Fibrosis, regardless of the cause, is characterized by extracellular matrix deposition, of which collagen type I is the major constituent. The progressive accumulation of connective tissue results in destruction of normal tissue architecture and internal organ failure. In both SSc and cGvHD, the severity of skin and internal organ fibrosis correlates with the clinical course of the disease (Pines et al., 2003, Biol. Blood Marrow Transplant 9:417-25).

SSc fibroblasts expressed increased levels of TGF(beta)RI and TGF(beta)RII protein and mRNA, as well as increased levels of type I collagen protein and alpha2(I) collagen mRNA. The half-lives of TGF(beta)RI and TGF(beta)RII mRNA in SSc fibroblasts did not change compared with those in control dermal fibroblasts, however the promoter activities of both genes were both significantly increased in SSc fibroblasts. These results suggest that increased levels of TGF(beta)RI and II in SSc fibroblasts play a role in excessive collagen production, and that up-regulation of TGF(beta)R expression might occur at the transcriptional level. Protein kinase C and/or PI 3-kinase might contribute to the up-regulation of TGF(beta)R expression in SSc fibroblasts. (Yamane et al., 2002, Arthritis Rheum. 46:2421-8).

The genesis of elastic fibers in early development involves deposition of tropoelastin (the soluble precursor of mature elastin) on a preformed template of fibrillin-rich microfibrils. Mature elastic fibers are thus a composite biomaterial comprising an outer microfibrillar mantle and an inner core of amaorphous crosslinked elastin. Fibrillins and fibrillin-rich microfibrils are conserved among invertebrates and vertebrates (Reber-Muller et al., 1995, Dev. Biol. 169:662-72). Tropoelastin evolved more recently to reinforce the high-pressure closed circulatory systems of higher vertebrates. The distribution of microfibrils in dynamic elastic tissues such as blood vessels, lung, ligaments and skin implies a central biomechanical role. Microfibrils are also abundant in some flexible tissues that do not express elastin e.g ciliary zonules that hold the lens in dynamic suspension (Ashworth et al., 1999, Biochem. J. 340:171-81), which emphasizes their independent evolutionary function.

The biology of elastic fibers is complex because of their multiple components, tightly regulated developmental pattern of deposition, multi-step hierarchical assembly, unique elastomeric properties and influence on cell phenotype.

Elastic fibers are found in the extracellular matrix of connective tissue, providing elasticity and resilience to tissues that are deformed repetitively and reversibly. Fibers are organised into distinct morphologies in different tissues: small, rope-like networks in lung, skin and ligament; thin concentric sheets in blood vessels; and large three-dimensional honeycomb structures in elastic cartilage (Vrhovski et al., 1998, Eur. J. Biochem. 258:1-18). Elastin is an extremely insoluble protein due to the extensive cross-linking at Lys residues. The cross-linking is preceded by selective lysine oxidation by the enzyme lysyl oxidase to produce α-amino adipic, 5-semialdehyde. Elastin is found in all vertebrates studied except the primitive cyclostomes, but has not been identified in invertebrates.

Various acquired and inherited diseases are known to affect the structure, distribution and abundance of elastic fibers. The organs most obviously affected are those rich in elastin. Due to the complexity of the elastic fiber and the interplay of an ensemble of molecules in fiber formation and structure, most of these diseases do not involve elastin as the primary defect; yet severely affect the elastic fiber integrity.

Elastic fibers are designed to maintain elastic function for a lifetime. However, various enzymes (matrix metalloproteinases and serine proteases) are able to cleave elastic fiber molecules (Kielty et al., 1994, FEBS Lett 351:85-9). Indeed, loss of elasticity due to degenerative changes is a major contributing factor in aging of connective tissues, in the development of aortic aneurysms and emphysema, and in degenerative changes in sun-damaged skin (Watson et al., 1999, J. Invest. Dermatol. 112: 782-7). The importance of elastic fibers is further highlighted by the severe heritable connective tissue diseases caused by mutations in components of elastic fibers (Milewicz et al., 2000, Matrix Biol. 19:471-80; Robinson et al., 2000, J. Med. Genet. 37:9-25). Fibrillin-1 mutations cause Marfan syndrome, which is associated with cardiovascular, ocular and skeletal defects. Fibrillin-2 mutations cause congenital contactural arachnodactyly (CCA) with overlapping skeletal and ocular symptoms, and elastin mutations cause Williams syndrome, supravalvular stenosis (SVAS) and cutis laxa (Tassabehji et al., 1998). (Le Saux et al., 2000, Nat. Genet. 25:223-7). Pseudoxanthoma elasticum (PXE) a heritable disease associated with elastic fiber calcification, was linked to mutations in an ion channel protein (Struk et al., 2000, J. Mol. Med. 78:282-6; Le Saux et al., 2000, Nat. Genet. 25:223-7; Ringpfeil et al., 2001, Exp. Dermatol. 10:221-8).

Abnormal accumulation of elastin fibers is seen in pseudoxanthoma elasticum and Buschke-Ollendorff syndrome, while an increase in fragmentation and loss of fibers is observed in cutis laxa, Marfan syndrome and Menkes disease. Acquired diseases include emphysema, where an increased degradation of elastic fibers is seen in the lung, and atherosclerosis, where a loss of elasticity in major blood vessels is accompanied by calcium and lipid deposition. Elastin destruction is modulated by proteases such as the matrix metalloproteinases and other elastases. Some of these diseases have been linked to errors in copper metabolism, and hence to lysyl oxidase, or to errors in microfibrillar proteins. Thus, an alteration in one of many key molecules involved in elastic fiber synthesis can result in severe damage to the entire fiber and organ system affected. A more complete understanding of elastic fiber biosynthesis and function has the potential to shed light on these diseases and lead to possible therapies. Due to the extreme insolubility of elastin, research into the process of elastic fiber formation was hampered until the discovery of the soluble precursor, tropoelastin, which was first isolated from copper-deficient animals.

Expression of tropoelastin mRNA and elastic fiber synthesis is highest in early development and occurs primarily within a limited period during development, as demonstrated in chick aorta, human skin fibroblasts, and sheep nuchal ligament and rat lung. The changes in elastin synthesis appear to be a consequence of both changes in proportion and amount of elastin mRNA and a strong correlation exists between mRNA levels and tropoelastin synthesis. This indicates that tropoelastin expression is mainly under pre-translational control and both pre and post-transcriptional control mechanisms have been described.

Age-dependence expression from the human elastin promoter has been demonstrated in mice in vivo. In chick aorta cells, the decrease in elastin synthesis that occurs with age results partly from mRNA destabilisation. Growth factors and hormones such as transforming growth factor, insulin-like growth factor I, vitamin D and interleukin-1 have all affect tropoelastin synthesis at either the promoter level or post-transcriptionally by affecting the stability of tropoelastin mRNA. In addition, there is evidence that tropoelastin may be under negative feedback autoregulation whereby accumulation of tropoelastin in the extracellular matrix space may inhibit the further production of tropoelastin mRNA.

Tropoelastin undergoes very little post-translational modification and there is no evidence for glycosylation. Hydroxylation of Pro residues occurs to a variable degree with 0-20% of the total Pro hydroxylated by the enzyme prolyl hydroxylase. It appears that Pro hydroxylation is not necessary for elastic fiber synthesis and that overhydroxylation may be detrimental. Inhibition of prolyl hydroxylase does not affect tropoelastin secretion but overhydroxylation caused by the addition of ascorbate, a cofactor of prolyl hydroxylase, to cell cultures resulted in a decrease in elastin production. It has been proposed that the effect of ascorbate may be due to transcriptional regulation of elastin mRNA levels, although the mechanism is not known. Overhydroxylation may result in destabilisation of tropoelastin secondary structure, thus inhibiting coacervation and decreasing the ability of tropoelastin to form fibers at physiological temperature. Cross-linking and the formation of insoluble elastin is consequently also reduced. Hydroxylation may be a byproduct of collagen hydroxylation, which occurs in the same cellular compartment. Alternatively, the presence of hydroxyproline may be a simple consequence of minor collagen contamination of tropoelastin preparations.

Deposition of tropoelastin into the extracellular space occurs only at specific regions on the cell surface, and tropoelastin is rapidly incorporated into the forming elastic fiber without further proteolysis. Before any elastin is deposited, microfibrils are secreted into the extracellular space close to the cell surface, marking the first step in elastogenesis. The relative elastin content increases as elastin is laid down in small clumps, which gradually fuse to form amorphous fibers.

Recently, the existence of other intracellular tropoelastin-binding proteins has been demonstrated. An endoplasmic reticulum chaperone, BiP, and FKBP65, a member of the immunophilin family with peptidyl prolyl cis-trans isomerisation ability co-immunoprecipitate with tropoelastin and may be important for proper folding of tropoelastin. Their roles have not yet been elucidated but are likely to be distinct from that of EBP.

Tropoelastin is soluble in cold aqueous solutions of less than 20° C. However, on raising the temperature towards the physiological range the solution becomes cloudy as the tropoelastin molecules aggregate by interactions between hydrophobic domains, such as the oligopeptide repetitive sequences, GVGVP, GGVP and GVGVAP, in a process termed coacervation.

Coacervation of tropoelastin is considered to be an important step in fibrillogenesis and it has been suggested that coacervation both concentrates and aligns tropoelastin molecules prior to cross-linking. There is evidence from circular dichroism (CD) studies that coacervate formation of tropoelastin and a-elastin (an oxalic acid-solubilised derivative of elastin) is an ordering process whereby polypeptide molecules are converted from a state of very little order to a conformation typical of substantial levels of structure. Inappropriate tropoelastin coacervation may be detrimental to fiber formation and it appears that many different molecules may influence this process.

After secretion into the extracellular space, tropoelastin is rapidly rendered insoluble by cross-link formation without any further modifications or proteolytic processing. The initial reaction is an oxidative deamination of Lys residues by the enzyme lysyl oxidase to produce allysine, also known as a-amino adipic (5-semialdehyde). All subsequent reactions are spontaneous and involve the condensation of closely positioned Lys and allysine residues to produce cross-links such as allysine aldol, lysinonorleucine, merodesmosine, and tetrafunctional cross-links unique to elastin, such as desmosine and isodesmo-sine. Tetrafunctional desmosine and isodesmosine are thought to result from two different pathways. Lysyl oxidase is a copper-dependent, highly thermostable enzyme with a broad pH optimum. It initiates cross-link formation in both collagen and elastin. When lysyl oxidase is inhibited, cross-linking is greatly reduced and tropoelastin accumulates in tissues, demonstrating the vital importance of this enzyme in elastogenesis. Nutritional deprivation of copper in humans and animals can lead to haemorrhage and aortic aneurysms. This is the basis for most tropoelastin purification protocols; animals are either fed copper-deficient diets, thereby reducing lysyl oxidase activity, or lysyl oxidase is inhibited irreversibly by lathyrogens such as aminopropionitrile. The affinity of lysyl oxidase is higher for insoluble forms of tropoelastin and collagen than for monomers in solution, emphasising the importance of tropoelastin coacervation for subsequent biosynthetic events. Lysyl oxidase has been localised to the mature elastic fiber and it may be incorporated into the growing fiber. Most of the Lys residues in tropoelastin are incorporated into cross-links.

Desmosine and isodesmosine are formed from four Lys residues but only link two tropoelastin chains. Three allysines and one Lys residue contribute to each desmosine and isodesmosine. It is thought that the presence of an aromatic residue (Tyr or Phe) on the C-terminal side of Lys prevents oxidation by lysyl oxidase. This favours lysinonorleucine formation and thus directing desmosine and isodesmosine formation. Lys residues in Ala-rich regions are always in groups of two or three separated by either two or three Ala residues. These regions are likely to be a-helical and the separation of Lys by two or three Ala residues places the Lys residues near one another on the same side of the helix, resulting in a conformation favourable to desmosine and isodesmosine formation. Only two exons, 19 and 25, contain three Lys residues instead of two. These exons are significant in that three separate tropoelastin chains are joined using these domains. Exons 19 and 25 of two antiparallel chains are joined by a desmosine and exon 10 from a third tropoelastin chain bridges them through two lysinonorleucine cross-links utilising the remaining two Lys residues. The Lys residues in Pro-containing domains, which dominate the N-terminal half of tropoelastin, are unlikely to be a-helical and hence unlikely to form desmosine or isodesmosine. However, their specific structures and interactions have not been determined.

Insoluble elastin has a very slow turnover in normal tissues. In adult rat lung, turnover is estimated to be several years, approaching the lifetime of the organism; this also appears to be the case in the human. One of the reasons for this may be the high resistance of elastin to proteolytic degradation. The main group of proteases able to degrade insoluble elastin is collectively known as elastases and they are generally active on a large number of substrates besides elastin. The most abundant mammalian serine elastases include pancreatic elastase; polymorphonuclear leukocyte elastase (also known as neutrophil elastase) and cathepsin G. Blood monocytes also produce elastolytic matrix metalloproteinases, which include 92 kDa and 72 kDa gelatinases, matrilysin and macrophage elastase. Blood monocytes produce serine elastases but after differentiation to macrophages lose this ability and instead produce matrix metalloproteinases. An important regulator of serine elastase function, particularly in lung, is al-proteinase inhibitor.

Elastin degradation is important in many physiological processes such as growth, wound healing, pregnancy and tissue remodelling. However, inappropriate and uncontrolled elastolysis can be destructive, contributing to disorders such as emphysema in the lung and atherosclerosis in arteries. Elastolysis in arteries can be enhanced by lipids and cholesterol. Increased elastolytic activity has also been observed in skin disorders such as cutis laxa. Increased elastolysis and degradation of elastin is also a feature of normal ageing.

Repair of protease-damaged elastin can occur but does not appear to produce elastin of the same quality as when originally laid down during growth. For example, in the repair of lung tissue after experimentally induced emphysema, elastin levels can return to normal but the new elastic fibers are highly disorganised and not fully functional. Some reutilisation of elastin peptides appears to occur during repair. Rather than the complete degradation of damaged elastin and resynthesis of new fibers, the repair mechanism appears to include the reduplication and reutilisation of peptides in the fibers.

Tropoelastin is far more vulnerable than elastin to proteolysis. Purification of tropoelastin from tissues usually results in extensive degradation, which can be substantially reduced by using protease inhibitors, particularly of serine proteases. Specific degradation by metalloproteinase has also been noted in cell cultures of smooth muscle cells. Even highly purified tropoelastin has been reported to degrade into approximately five discrete bands on prolonged storage, leading to a hypothesis that it is co-purified with an intrinsic protease, which gradually breaks it down. During purification of recombinant tropoelastin specific degradation products are occasionally observed, similar to that seen after purification from tissue. Mammalian serum contains proteases, which are capable of degrading tropoelastin. Serum has also been shown to induce elastase activity in smooth muscle cells leading to degradation of elastin. Serine protease inhibitors can reduce the degradation of tropoelastin caused by serum.

Various hypotheses have been put forward as to the possible role and consequences of tropoelastin degradation. Serine proteases, in particular plasmin, modulate tropoelastin mRNA levels by suggesting that soluble tropoelastin accumulation acts as a negative feedback control mechanism for transcription. Soluble peptides produced by degradation of elastin with elastase have been demonstrated to down-regulate mRNA levels when added to undigested elastin-producing cultures, while increasing mRNA levels in damaged cultures, thus serving to localise repair to damaged tissues. Soluble elastin peptides can cause vasodilation and are chemo-attractants for monocytes and fibroblasts. This suggests that protease degradation products derived from cross-linked material play a role in cell migration and inflammation. Thus, the proteolytic degradation of tropoelastin and elastin may have important consequences for normal elastogenesis and repair processes.

The amino acid lysine is an essential amino acid in mammals, and a biochemical path exists to recover lysine so that it can be reused. Brown et. al. in U.S. Pat. No. 6,006,958, incorporated by reference and recited in its entirety herein, teaches that lysine is enzymatically recovered from fructoselysine with the concomitant production of 3 deoxyglucosone (3DG) in the Amadori Pathway. 3DG and the enzyme are also found in skin as taught in International Publication Number WO 03/089601, having an International Patent Application number of PCT/US03/12003, incorporated by reference and recited in its entirety herein. Lysine becomes glycated in the body as a result of a reversible reaction between glucose and the ε-NH2 groups of lysine-containing proteins. This process proceeds via a Schiff base intermediate which rearranges to the more stable fructoselysine (FL), an “Amadori product.” Cooked animals products introduced by diet can also contribute glycated protein. Glycated protein is eventually degraded resulting in fructoselysine (FL). Fructoseamine-3-Kinase (F3K) phosphorylates FL on its 3′-OH creating fructoselysine-3-phosphate (FL3P) which then spontaneously decomposes into lysine, Pi, and 3DG. Thus F3K allows the body to recover lysine.

Brown et al., U.S. Pat. No. 6,004,958 and International Publication Number WO 03/089601, having an International Patent Application number of PCT/US03/12003, describe compounds which inhibit the enzymatic conversion of fructoselysine to FL3P, inhibit the formation of lysine from the deglycation of fructoselysine (FL), inhibit the formation of 3DG, as well as provide for the inactivation of 3DG and detoxification of 3DG. Specific compounds which are representative of the class have also been described (Brown et al., International Publication No. WO 98/33492). For example, it was found that urinary or plasma 3DG can be reduced by meglumine, sorbitollysine, mannitollysine, and galactitollysine. Id. It was also found that diets high in glycated protein are harmful to the kidney and cause a decrease in birth rate. Id. It has also been disclosed that the fructoselysine pathway is involved in kidney carcinogenesis. Id. Further, previous studies demonstrate that diet and 3DG can play a role in carcinogenesis associated with this pathway (see International Publication Nos. WO 00/24405; WO 00/62626; WO 98/33492).

3DG is a highly reactive molecule that can be detoxified in the body by at least two pathways. In one pathway, 3DG is reduced to 3-deoxyfructose (3DF) by aldehyde reductase, and the 3DF is then efficiently excreted in urine (Takahashi et al., 1995, Biochemistry 34:1433-8). Another detoxification reaction oxidizes 3DG to 3-deoxy-2-ketogluconic acid (DGA) by oxoaldehyde dehydrogenase (Fujii et al., 1995, Biochem. Biophys. Res. Commun. 210:852-7).

Results of studies to date show that one of these enzymes, aldehyde reductase, is adversely affected in diabetes. When isolated from diabetic rat liver, this enzyme is glycated on lysine at positions 67, 84 and 140 and has a low catalytic efficiency when compared with the normal, unmodified enzyme (Takahashi et al., 1995, Biochemistry 34:1433-8). Since diabetic patients have higher ratios of glycated proteins than normoglycemic individuals, they are likely to have both higher levels of 3DG and a reduced ability to detoxify this reactive molecule by reduction to 3DF. It has also been found that overexpression of aldehyde reductase protects PC12 cells from the cytotoxic effects of methylglyoxal or 3DG (Suzuki et al., 1998, J. Biochem. 123:353-7).

The mechanism by which aldehyde reductase works has been studied. These studies demonstrated that this important detoxification enzyme is inhibited by aldose reductase inhibitors (ARIs) (Barski et al., 1995, Biochemistry 34:11264-75). ARIs are currently under clinical investigation for their potential to reduce diabetic complications. These compounds, as a class, have shown some effect on short-term diabetic complications, but they lack clinical effect on long-term diabetic complications and they worsen kidney function in rats fed a high protein diet. This finding is consistent with the newly discovered metabolic pathway for lysine recovery.

Aminoguanidine (AG), an agent that detoxifies 3DG pharmacologically via formation of rapidly excreted covalent derivatives (Hirsch et al., 1992, Carbohydr. Res. 232:125-30), has been shown to reduce AGE-associated retinal, neural, arterial, and renal pathologies in animal models (Brownlee, 1994, Diabetes 43:836-41; Brownlee et al., 1986, Science 232:1629-32; Ellis et al., 1991, Metabolism 40:1016-9; Soulis-Liparota et al., 1991, Diabetes 40:1328-34, and Edelstein et al., 1992, Diabetologia 35:96-7).

Past studies have concentrated on the role of 3DG in diabetes. It has been demonstrated that diabetic humans have detectably elevated levels of 3DG and 3-deoxyfructose (3DF), 3DG's detoxification product, in plasma (Niwa et al., 1993, Biochem. Biophys. Res. Commun. 196:837-43; Wells-Knecht et al., 1994, Diabetes 43:1152-6) and in urine (Wells-Knecht et al., 1994, Diabetes 43:1152-6), as compared with non-diabetic individuals. Furthermore, diabetics with nephropathy were found to have elevated plasma levels of 3DG compared to non-diabetics (Niwa et al., 1993, Biochem. Biophys. Res. Commun. 196:837-43).

A recent study comparing patients with insulin-dependent diabetes mellitus (IDDM) and noninsulin-dependent diabetes mellitus (NIDDM) confirmed that 3DG and 3DP levels were elevated in blood and urine from both types of patient populations. Thus the normal pathway for reductive detoxification of 3DG (conversion to 3DF) may be impaired in diabetic humans [Lal et al., 1995, Arch. Biochem. Biophys. 318:191-9). It has even been shown that incubation of glucose and proteins in vitro under physiological conditions produces 3DG.

In turn, it has been demonstrated that 3DG glycates and crosslinks protein creating detectable AGE products (Baynes et al., 1984, Methods Enzymol. 106:88-98; Dyer et al., 1991, J. Biol. Chem. 266:11654-60).

Furthermore, elevated levels of 3DG-modified proteins have been found in diabetic rat kidneys compared to control rat kidneys (Niwa et al., 1997, J. Clin. Invest. 99:1272-80). It has been demonstrated that 3DG has the ability to inactivate enzymes such as glutathione reductase, a central antioxidant enzyme. It has also been shown that hemoglobin-AGE levels are elevated in diabetic individuals (Makita et al., 1992, Science 258:651-3) and other AGE proteins have been shown in experimental models to accumulate with time, increasing from 5-50 fold over periods of 5-20 weeks in the retina, lens and renal cortex of diabetic rats (Brownlee, 1994, Diabetes 43:836-41). In addition, it has been demonstrated that 3DG is a teratogenic factor in diabetic embryopathy (Eriksson et al., 1998, Diabetes 47:1960-6).

Nonenzymatic glycation, in which reducing sugars are covalently attached to free amino groups and ultimately form AGEs, has been found to occur during normal aging and to occur at an accelerated rate in diabetes mellitus (Bierhaus et al., 1998, Cardiovasc. Res. 37:586-600). Crosslinking of proteins and the subsequent AGE formation are irreversible processes that alter the structural and functional properties of proteins, lipid components, and nucleic acids (Bierhaus et al., 1998, Cardiovasc. Res. 37:586-600). These processes have been postulated to contribute to the development of a range of diabetic complications including nephropathy, retinopathy, and neuropathy (Rahbar et al., 1999, Biochem. Biophys. Res. Commun. 262:651-6).

It has been demonstrated that inhibition of AGE formation reduced the extent of nephropathy in diabetic rats (Ninomiya et al., 2001, Diabetes 50:A178-179). Therefore, substances that inhibit AGE formation and/or oxidative stress appear to limit the progression of diabetic complications and may offer new tools for therapeutic interventions in the treatment of diabetes [Thornalley, 1996, Endocrinol. Metab. 3:149-166; Bierhaus et al., 1998, Cardiovasc. Res. 37:586-600).

Finally, a direct link between serum levels of 3DG indiabetics and the risk of development of diabetic complications has been demonstrated (Kusunoki et al., 2003, Diabetes Care 26:1889-94). The results show that the fasting serum 3DG level is elevated in diabetic patients and that the patients with relatively higher 3DG levels were prone to suffer from more severe complications, indicating a possible association of 3DG with diabetic microangiopathy.

In summary, 3DG has numerous toxic effects on cells and is present in elevated levels in several disease states. The harmful effects of 3DG include, but are not limited to, the following.

It is known that 3DG induces reactive oxygen species in human umbilical vein endothelial cells, which results in oxidative DNA damage (Shimoi et al., 2001, Mutat. Res. 480-481:371-8). Prior studies indicate that 3DG inactivates aldehyde reductase (Takahashi et al., 1995, Biochemistry 34:1433-8). This is important, since aldehyde reductase is the cellular enzyme that protects the body from 3DG. There is supportive evidence that this detoxification of 3DG to 3-deoxyfructose (3DF) is impaired in diabetic humans since their ratio of urinary and plasma 3DG to 3DF differs significantly from non-diabetic individuals (Lai et al., 1997, Arch. Biochem. Biophys. 342:254-60).

Additionally, it has been demonstrated that 3DG induced reactive oxygen species contribute to the development of diabetic complications (Araki, 1997, Nippon Ronen Igakkai Zasshi 34:716-20). Specifically, 3DG induces heparin-binding epidermal growth factor, a smooth muscle mitogen that is abundant in atherosclerotic plaques. This suggests that an increase in 3DG may trigger atherogenesis in diabetes (Taniguchi et al., 1996, Diabetes 45 Suppl. 3:S81-3; Che et al., 1997, J. Biol. Chem. 272:18453-9).

Further, 3DG is a known teratogenic factor in diabetic embryopathy leading to embryo malformation (Eriksson et al., 1998, Diabetes 47:1960-6). This appears to arise from 3DG accumulation, which leads to superoxide-mediated embryopathy.

More recently, it was demonstrated that 3DG induces apoptosis in macrophage-derived cell lines [Okado et al., 1996, Biochem. Biophys. Res. Commun. 225:219-24), and is toxic to cultured cortical neurons (Kikuchi et al., 1999, J. Neurosci. Res. 57:280-9) and PC12 cells (Suzuki et al., 1998, J. Biochem. 123:353-7). A recent study on the cause of amyotropic lateral sclerosis, a form of motor neuron disease, has suggested that accumulation of 3DG can lead to neurotoxicity as a result of ROS generation (Shinpo et al., 2000, Brain Res. 861:151-9).

Previous studies demonstrated that 3DG glycates and crosslinks protein leading to a complex mixture of compounds called advanced glycation end products (AGEs) (Baynes et al., 1984, Methods Enzymol. 106: 88-98; Dyer et al., 1991, J. Biol. Chem. 266:11654-60). AGEs have been implicated in most inflammatory diseases such as diabetes, atherosclerosis and dementia. They are most commonly formed on long-lived structural proteins such as collagen.

Hemoglobin-AGE levels are elevated in diabetic individuals (Makita et al., 1992, Science 258:651-3), and other AGE proteins have been shown in experimental models to accumulate with time, increasing from 5-50 fold over periods of 5-20 weeks in the retina, lens and renal cortex of diabetic rats (Brownlee, 1994, Diabetes 43:836-41).

AGEs have specific receptors on cells called RAGE. The activation of cellular RAGE on endothelium, mononuclear phagocytes, and lymphocytes triggers the generation of free radicals and the expression of inflammatory gene mediators (Hofmann et al., 1999, Cell 97:889-901). This increased oxidative stress leads to the activation of the transcription factor NF-kB and promotes the expression of NF-kB genes that have been associated with atherosclerosis (Bierhaus et al., 1998, Cardiovasc. Res. 37:586-600).

In relationship to cancer, blockage of RAGE activation inhibits several mechanisms linked to tumor proliferation and trans-endothelial migration of tumor cells. This also decreases growth and metastases of both spontaneous and implanted tumors (Taguchi et al., 2000, Nature 405:354-60).

Diabetic humans have elevated levels of 3DG and 3DF in plasma (Niwa et al., 1993, Biochem. Biophys. Res. Commun. 196:837-43; Wells-Knecht et al., 1994, Diabetes 43:1152-6) and urine (Niwa et al., 1993, Biochem. Biophys. Res. Commun. 196:837-43; Wells-Knecht et al., 1994, Diabetes 43:1152-6), as compared with non-diabetic individuals.

Diabetics with nephropathy were found to have elevated plasma levels of 3DG compared with other diabetics (Niwa et al., 1993, Biochem. Biophys. Res. Commun. 196:837-43). Elevated levels of 3DG-modified proteins are found in diabetic versus control rat kidneys (Niwa et al., 1997, J. Clin. Invest. 99:1272-80). In addition, the fasting serum 3-DG level is elevated in diabetic patients and that the patients with relatively higher 3-DG levels were prone to suffer from more severe complications, indicating a possible association of 3-DG with diabetic microangiopathy (Kusunoki et al., 2003, Diabetes Care 26:1889-94).

To date, no one has identified a useful or promising method of intervention for regulation for collagen or elastin in mammals, and in particular, in humans. Therefore, the role of the regulation of collagen and elastin levels in connective tissue-related diseases, disorders, or conditions has not been elucidated. There is a long-felt need to identify methods of treating and/or alleviating such disease states, such as diabetes. Further, skin aging, wrinkling, and the like, are the subject of much research and there is a long felt need in the art for the development of new methods to treat wrinkling or aging skin, as well as diseased skin. The present invention satisfies these needs.

### BRIEF SUMMARY OF THE INVENTION

The present invention includes a method of decreasing desmosine levels in a mammal in need thereof, the method comprising administering to a mammal a composition comprising an inhibitor of the Amadorase pathway. In one embodiment, the inhibitor inhibits fructoseamine kinase. In another embodiment, the composition further comprises an inhibitor of 3DG. In one aspect, the mammal is a human. In another aspect, a human has at least one disease selected from the group consisting of diabetes and lung fibrosis.

The invention also-includes a method of stabilizing desmosine levels in a mammal in need thereof, comprising administering to the mammal a composition comprising an inhibitor of the Amadorase pathway. In one embodiment, the composition comprises an inhibitor of fructoseamine kinase. In another embodiment, the composition further comprises an inhibitor of 3DG.9. In one aspect, the mammal is a human. In another aspect, a human has at least one disease selected from the group consisting of diabetes and lung fibrosis.

In one embodiment of a method of the invention, the desmosines are in at least one of the locations selected from the group consisting of the extracellular matrix, lung, kidney, skin, heart, arteries, ligament and elastic cartilage.

In another embodiment of a method of the invention, an inhibitor of fructoseamine kinase is administered to a mammal via a route selected from the group consisting of topical, oral, rectal, vaginal, intramuscular, subcutaneous, and intravenous.

In yet another embodiment of a method of the invention, an inhibitor of fructoseamine kinase is an antibody.

In still another embodiment of a method of the invention, the fructoseamine kinase is encoded by a nucleic acid comprising a nucleic acid encoding the amino acid sequence set forth in SEQ ID NO:2.

In one embodiment of the present invention, a method of decreasing desmosine levels in a mammal in need thereof comprises administering to the mammal a composition comprising an inhibitor of the Amadorase pathway, wherein the inhibitor is a compound comprising the formula of formula XIX:

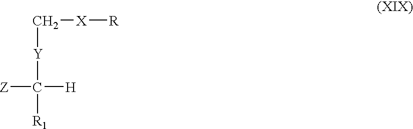

a. wherein X is —NR′—, —S(O)—, —S(O)2—, or —O—, R′ being selected from the group consisting of H, linear or branched chain alkyl group (C1-C4), CH2(CHOR2)nCH2OR2 where n=1-5 and R2 is H, alkyl (C1-C4) or an unsubstituted or substituted aryl group (C6-C10) or aralkyl group (C7-C10), CH(CH2OR2)(CHOR2)nCH2OR2 where n=1-4 and R2 is H, alkyl (C1-C4) or an unsubstituted or substituted aryl group (C6-C10) or aralkyl group (C7-C10), an unsubstituted or substituted aryl group (C6-C10), and an unsubstituted or substituted aralkyl group (C7-C10);

b. R is a substituent selected from the group consisting of H, an amino acid residue, a polyaminoacid residue, a peptide chain, a linear or branched chain aliphatic group (C1-C8), which is unsubstituted or substituted with at least one nitrogen- or oxygen-containing substituent, a linear or branched chain aliphatic group (C1-C8), which is unsubstituted or substituted with at least one nitrogen- or oxygen-containing substituent and interrupted by at least one —O—, —NH—, or —NR″— moiety;

c. R″ being linear or branched chain alkyl group (C1-C6) and an unsubstituted or substituted aryl group (C6-C10) or aralkyl group (C7-C10), with the proviso that when X represents —NR′—, R and R′, together with the nitrogen atom to which they are attached, may also represent a substituted or unsubstituted heterocyclic ring having from 5 to 7 ring atoms, with at least one of nitrogen and oxygen being the only heteroatoms in said ring, said aryl group (C6-C10) or aralkyl group (C7-C10) and said heterocyclic ring substituents being selected from the group consisting of H, alkyl (C1-C6), halogen, CF3, CN, NO2 and —O-alkyl (C1-C6); R1 is a polyol moiety having 1 to 4 linear carbon atoms, Y is a hydroxymethylene moiety —CHOH—; Z is selected from the group consisting of —H, —O-alkyl (C1-C6), -halogen —CF3, —CN, —COOH, and —SO3H2, and optionally —OH;

d. The isomers and pharmaceutically acceptable salts of the compound, except that X-R in the above formula does not represent hydroxyl or thiol.

In one aspect of the invention, the composition comprises the inhibitor from about 0.0001% to about 15% by weight. In another aspect, the composition is a pharmaceutical composition.

In another aspect of the invention, the compound comprising formula XIX is selected from the group consisting of galactitol lysine, 3-deoxy sorbitol lysine, 3-deoxy-3-fluoro-xylitol lysine, 3-deoxy-3-cyano sorbitol lysine, 3-O-methyl sorbitollysine, meglumine, sorbitol lysine and mannitol lysine. In yet another aspect, the compound is 3-O-methyl sorbitollysine.

In one embodiment, the present invention features a method of decreasing the level of mRNA for collagen in a mammal by increasing the flux through the Amadori pathway in the mammal, comprising administering to the mammal a compound comprising formula XIX(b)

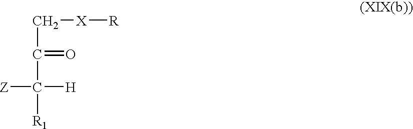

a. wherein X is —NR′—, —S(O)—, —S(O)2—, or —O—, R′ being selected from the group consisting of H or a guanidine group, linear or branched chain alkyl group (C1-C4), CH2(CHOR2)nCH2OR2 where n=1-5 and R2 is H, alkyl (C1-C4) or an unsubstituted or substituted aryl group (C6-C10) or araalkyl group (C7-C10), CH(CH2OR2)(CHOR2)nCH2OR2 where n=1-4 and R2 is H, alkyl (C1-C4) or an unsubstituted or substituted aryl group (C6-C10) or araalkyl group (C7-C10), an unsubstituted or substituted aryl group (C6-C10), and an unsubstituted or substituted aralkyl group (C7-C10);

b. R is a substituent selected from the group consisting of H, an amino acid residue, a polyaminoacid residue, a peptide chain, a linear or branched chain aliphatic group (C1-C8), which is unsubstituted or substituted with at least one nitrogen- or oxygen-containing substituent, a linear or branched chain aliphatic group (C1-C8), which is unsubstituted or substituted with at least one nitrogen- or oxygen-containing substituent and interrupted by at least one —O—, —NH—, or —NR″— moiety;

c. R″ being linear or branched chain alkyl group (C1-C6) and an unsubstituted or substituted aryl group (C6-C10) or aralkyl group (C7-C10), with the proviso that when X represents —NR′—, R and R′, together with the nitrogen atom to which they are attached, may also represent a substituted or unsubstituted heterocyclic ring having from 5 to 7 ring atoms, with at least one of nitrogen and oxygen being the only heteroatoms in said ring, said aryl group (C6-C10) or aralkyl group (C7-C10) and said heterocyclic ring substituents being selected from the group consisting of H, alkyl (C1-C6), halogen, CF3, CN, NO2 and —O-alkyl (C1-C6); R1 is a polyol moiety having 1 to 4 linear carbon atoms, Z is selected from the group consisting of —H, —O-alkyl (C1-C6), -halogen —CF3, —CN, —COOH, and —SO3H2, and optionally —OH;

d. the isomers and pharmaceutically acceptable salts of the compound, except that X-R in the above formula does not represent hydroxyl or thiol.

In one aspect of the invention, the collagen is Type I collagen. In another aspect, the compound is a substrate for fructoseamine kinase. In one aspect, the compound is fructoselysine.

In one embodiment, the present invention features a method of treating scleroderma in a mammal, comprising administering to the mammal a composition comprising a compound that increases the flux through the Amadorase Pathway in the mammal, thereby decreasing the levels of mRNA for collagen Type I.

In another embodiment, the present invention features a method of treating keloids in a mammal, the method comprising administering to the mammal a composition comprising a compound that increases the flux through the Amadorase Pathway in said mammal, thereby decreasing the levels of mRNA for collagen Type I. In one aspect, the compound stimulates fructoseamine kinase. In another aspect, the compound is selected from the group consisting of fructose lysine 3 phosphate and an analog of fructose lysine 3 phosphate.

In one embodiment, the present invention features a method of treating scleroderma in a mammal, comprising the administration to the mammal of a composition comprising a first compound that stimulates the flux through the Amadorase pathway and a second compound that inactivates 3DG. In one aspect, the second compound is structural formula I: 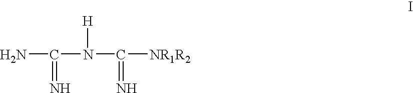
wherein R1 and R2 are independently selected from the group consisting of a hydrogen, a lower alkyl, a lower alkoxy and an aryl group; or wherein said R1 and said R2 together with a nitrogen atom form a heterocyclic ring containing from 1 to 2 heteroatoms and 2 to 6 carbon atoms, the second of said heteroatoms comprising nitrogen, oxygen, or sulfur; further wherein said lower alkyl group is selected from the group consisting of 1 to 6 carbon atoms; wherein said lower alkoxy group is selected from the group consisting of 1 to 6 carbon atoms; and wherein said aryl group comprises substituted and unsubstituted phenyl and pyridyl groups.

The present invention also features a method of inhibiting the reaction of at least one dicarbonyl compound with tropoelastin in a mammal, comprising administering to the mammal an effective amount of an inhibitor of an alpha-dicarbonyl sugar function. In one aspect, the dicarbonyl compound is 3DG. In another aspect, the inhibitor chelates 3DG. In yet another aspect, the inhibitor detoxifies 3DG. In an aspect of the invention, the inhibitor is selected from the group consisting of structural formulas I-XVII and XVIII. In another aspect of the invention, the inhibitor is structural formula I: 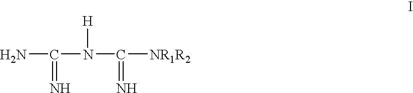
wherein R1 and R2 are independently selected from the group consisting of a hydrogen, a lower alkyl, a lower alkoxy and an aryl group; or wherein said R1 and said R2 together with a nitrogen atom form a heterocyclic ring containing from 1 to 2 heteroatoms and 2 to 6 carbon atoms, the second of said heteroatoms comprising nitrogen, oxygen, or sulfur; further wherein said lower alkyl group is selected from the group consisting of 1 to 6 carbon atoms; wherein said lower alkoxy group is selected from the group consisting of 1 to 6 carbon atoms; and wherein said aryl group comprises substituted and unsubstituted phenyl and pyridyl groups.

In another aspect of the invention, the compound is selected from the group consisting of N, N-dimethylimidodicarbonimidic diamide, imidodicarbonimidic diamide, N-phenylimidodicarbonimidic diamide, N-(aminoiminomethyl)-4-morpholinecarboximidamide, N-(aminoiminomethyl)-4-thiomorpholinecarboximidamide, N-(aminoiminomethyl)-4-methyl-1-piperazinecarboximidamide, N-(aminoiminomethyl)-1-piperidinecarboximidamide, N-(aminoiminomethyl)-1-pyrrolidinecarboximidamide, N-(aminoiminomethyl)-I-hexahydroazepinecarboximidamide, (aminoiminomethyl)-I-hexahydroazepinecarboximidamide, N-4-pyridylimidodicarbonimidic diamide, N, N-di-n-hexylimidodicarbonimidic diamide, N,N-di-n-pentylimidodicarbonimidic diamide, N,N-d-n-butylimidodicarbonimidic diamide, N,N-dipropylimidodicarbonimidic diamide, and N,N-diethylimidodicarbonimidic diamide.

In another aspect of the invention, the structural formula is structural formula II: 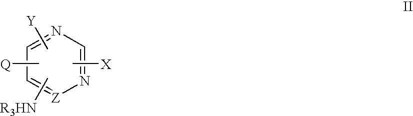
wherein Z is N or CH; wherein X, Y, and Q each independently is selected from the group consisting of a hydrogen, an amino, a heterocyclo, an amino lower alkyl, a lower alkyl, and a hydroxy group; further wherein R3 comprises a hydrogen or an amino group or their corresponding 3-oxides; wherein said lower alkyl group is selected from the group consisting of 1 to 6 carbon atoms; wherein said heterocyclic group is selected from the group consisting of 3 to 6 carbon atoms; and wherein X, Y, and Q can each be present as a hydroxy variant on a nitrogen atom.

In another aspect of the invention, the compound is selected from the group consisting of 4,5-diaminopyrimidine, 4-amino-5-aminomethyl-2-methylpyrimidine, 6-(piperidino)-2,4-diaminopyrimidine 3-oxide, 4,6-diaminopyrimidine, 4,5,6-triaminopyrimidine, 4,5-diamino-6-hydroxy pyrimidine, 2,4,5-triamino-6-hydroxypyrimidine, 2,4,6-triaminopyrimidine, 4,5-diamino-2-methylpyrimidine, 4,5-diamino-2,6-dimethylpyrimidine, 4,5-diamino-2-hydroxy-pyrimidine, and 4,5-diamino-2-hydroxy-6-methylpyrimidine.

In another aspect of the invention, the structural formula is structural formula III: 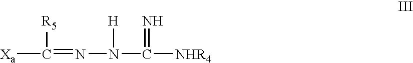
wherein R4 is hydrogen or acyl, R5 is hydrogen or lower alkyl, Xa is a substituent selected from the group consisting of a lower alkyl, a carboxy, a carboxymethyl, an optionally substituted phenyl and an optionally substituted pyridyl group, wherein said optional substituent is selected from the group consisting of a halogen, a lower alkyl, a hydroxy lower alkyl, a hydroxy, and an acetylamino group; further wherein, when X is a phenyl or pyridyl group, optionally substituted, R5 is hydrogen; and wherein, said lower alkyl group is selected from the group consisting of 1 to 6 carbon atoms.

In another aspect of the invention, the compound is selected from the group consisting of N-acetyl-2-(phenylmethylene)hydrazinecarboximidamide, 2-(phenylmethylene)hydrazinecarboximidamide, 2-(2,6-dichlorophenylmethylene)hydrazinecarboximidamide pyridoxal guanylhydrazone, pyridoxal phosphate guanylhydrazone, 2-(1-methylethylidene)hydrazinecarboximidamide, pyruvic acid guanylhydrazone, 4-acetamidobenzaldehyde guanylhydrazone, 4-acetamidobenzaldehyde N-acetylguanylhydrazone, and acetoacetic acid guanylhydrazone.

In another aspect of the invention, the structural formula is structural formula IV: 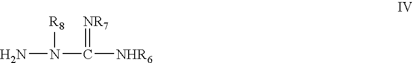
wherein, R6 is selected from the group consisting of a hydrogen, a lower alkyl group, and a phenyl group, further wherein said phenyl group is optionally substituted by a structure selected from the group consisting of a 1-3 halo, an amino, a hydroxy, and a lower alkyl group, wherein when said phenyl group is substituted, a point of said substitution is selected from the group consisting of an ortho, a meta, and a para point of attachment of said phenyl ring to a straight chain of said structural formula IV; R7 is selected from the group consisting of a hydrogen, a lower alkyl group, and an amino group; R8 is hydrogen or a lower alkyl group; further wherein said lower alkyl group is selected from a lower alkyl group consisting of 1 to 6 carbon atoms.

In another aspect of the invention, the compound is selected from the group consisting of equival n-butanehydrazonic acid hydrazide, 4-methylbenzamidrazone, N-methylbenzenecarboximidic acid hydrazide, benzenecarboximidic acid 1-methylhydrazide, 3-chlorobenzamidrazone, 4-chlorobenzamidrazone, 2-fluorobenzamidrazone, 3-fluorobenzamidrazone, 4-fluorobenzamidrazone, 2-hydroxybenzamidrazone, 3-hydroxybenzamidrazone, 4-hydroxybenzamidrazone, 2-aminobenzamidrazone, benzenecarbohydrazonic acid hydrazide, and benzenecarbohydrazonic acid 1-methylhydrazide.

In another aspect of the invention, the structural formula is structural formula V:

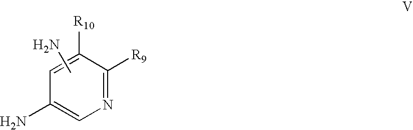

wherein R9 and R10 are independently selected from the group consisting of a hydrogen, a hydroxy, a lower alkyl, and a lower alkoxy, further wherein a “floating” amino group is adjacent to a fixed amino group; said lower alkyl group is selected from a lower alkyl group consisting of 1 to 6 carbon atoms; and said lower alkoxy group is selected from a lower alkoxy group consisting of 1 to 6 carbon atoms.

In another aspect of the invention, the compound is selected from the group consisting of 3,4-diaminopyridine, 2,3-diaminopyridine, 5-methyl-2,3-diaminopyridine, 4-methyl-2,3-diaminopyridine, 6-methyl-2,3-pyridinediamine, 4,6-dimethyl-2,3-pyridinediamine, 6-hydroxy-2,3-diaminopyridine, 6-ethoxy-2,3-diaminopyridine, 6-dimethylamino-2,3-diaminopyridine, diethyl 2-(2,3-diamino-6-pyridyl) malonate, 6 (4-methyl-l-pyperazinyl)-2,3-pyridinediamine, 6-(methylthio)-5 (trifluoromethyl)-2,3-pyridinediamine, 5-(trifluoromethyl)-2,3-pyridinediamine, 6-(2,2,2-trifluorethoxy)-5- (trifluoromethyl)-2,3-pyridinediamine, 6-chloro-5-(trifluoromethyl)-2,3-pyridinediamine, 5-methoxy-6-(methylthio)-2,3-pyridinediamine, pyridinediamine, 5-bromo4-methyl-2,3-pyridinediamine, 5-(trifluoromethyl-2,3-pyridinediamine, 6-bromo-4-methyl-2,3-pyridinediamine, 5-bromo-6-methyl-2,3-pyridinediamine, 6-methoxy-3,4-pyridinediamine, 2-methoxy-3,4-pyridinediamine, 5-methyl-3,4-pyridinediamine, 5-methoxy-3,4-pyridinediamine, 5-bromo-3,4-pyridinediamine, 2,3,4-pyridinetriamine, 2,3,5-pyridinetriamine, 4-methyl-2,3,6-pyridinetriamine, 4-(methylthio)-2,3,6-pyridinetriamine, 4-ethoxy-2,3,6-pyridinetriamine, 2,3,6-pyridinetriamine, 3,4,5-pyridinetriamine, 4-methoxy-2,3-pyridinediamine, 5-methoxy-2,3-pyridinediamine, and 6-methoxy-2,3-pyridinediamine.

In another aspect of the invention, the structural formula is structural formula VI: 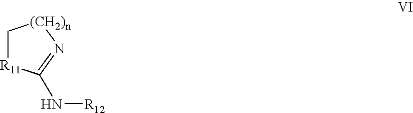
wherein n is 1 or 2, R 11is an amino group or a hydroxyethyl group, and R12 is selected from the group consisting of an amino group, a hydroxyalkylamino group, a lower alkyl group, and a group of the formula alk-Ya, further wherein alk is a lower alkylene group and Ya is selected from the group consisting of a hydroxy, a lower alkoxy group, a lower alkylthio group, a lower alkylamino group, and a heterocyclic group, wherein said heterocyclic group contains 4 to 7 ring members and 1 to 3 heteroatoms; further wherein, when said R11 is a hydroxyethyl group then said R12 is an amino group; said lower alkyl group is selected from the group consisting of 1 to 6 carbon atoms, said lower alkylene group is selected from the group consisting of 1 to 6 carbon atoms, and said lower alkoxy group is selected from the group consisting of 1 to 6 carbon atoms.

In another aspect of the invention, the compound is selected from the group consisting of 1-amino-2-[2-(2-hydroxyethyl) hydrazino]-2-imidazoline, 1-amino-[2-(2-hydroxyethyl) hydrazino]-2-imidazoline, 1-amino-2-(2-hydroxyethylamino)-2-imidazoline, 1-(2-hydroxyethyl)-2-hydrazino-1,4,5,6-tetrahydropyrimidine, I-(2-hydroxyethyl) 2-hydrazino-2-imidazoline, 1-amino-2-([2-(4-morpholino)ethyl]amino)imidazoline, ([2-(4-morpholino)ethyl]amino)imidazoline, 1 -amino-2-([3 -(4-morpholino) propyl]amino)imidazoline, 1 -amino-2-([3-(4-methylpiperazin-1-yl)propyl]-amino)imidazoline; 1-amino-2-([3-(dimethylamino)propyl] amino)imidazoline, 1-amino-2-[(3-ethoxypropyl)amino] imidazoline, 1-amino-2-([3-(1-imidazolyl)propyl] amino)imidazoline, 1-amino-2-(2-methoxyethylamino)-2-imidazoline, (2-methoxyethylamino)-2-imidazoline, 1-amino-2-(3-isopropoxypropylamino)-2-imidazoline, 1-amino-2-(3-methylthiopropylamino)-2-imidazoline, 1-amino-2 [3-(l-piperidino)propylamino)imidazoline, l-amino-2-[2, 2-dimethyl-3-(dimethylamino) propylamino]-2-imidazoline, and 1-amino-2-(neopentylamino)-2-imidazoline.

In another aspect of the invention, the structural formula is structural formula VII: 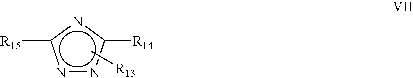
wherein, R13 is selected from the group consisting of a hydrogen and an amino group, R14 and R15 are independently selected from the group consisting of an amino group, a hydrazino group, a lower alkyl group, and an aryl group, further wherein, one of said R13, R14, and R15 must be an amino group or a hydrazino group; wherein said aryl group is selected from the group consisting of 6 to 10 carbon atoms, and said lower alkoxy group is selected from the group consisting of 1 to 6 carbon atoms.

In another aspect of the invention, the compound is selected from the group consisting of 3,4-diamino-5-methyl-1,2,4-triazole, 3,5-dimethyl-4H-1,2,4-triazol-4-amine, 4-triazol-4-amine, 4-triazol-4-amine, 4-triazol-4-amine, 2, 4-triazole-3,4-diamine, 5-(1-ethylpropyl)-4H-1,2,4-triazole-3,4-diamine, 5-isopropyl-4H-1,2,4-triazole-3,4-diamine, 5-cyclohexyl-4H-1,2,4-triazole-3,4-diamine, 5-methyl-4H-1,2,4-triazole-3,4-diamine, 5-phenyl-4H-1,2,4-triazole-3,4-diamine, 5-propyl-4H-1,2,4-triazole-3,4-diamine, and 5-cyclohexyl-4H-1,2,4-triazole-3,4-diamine.

In another aspect of the invention, the structural formula is structural formula VIII: 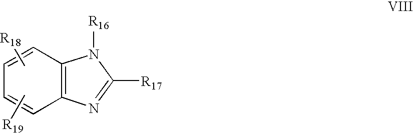
wherein, R16 is selected from the group consisting of a hydrogen-and an amino group; R17 is selected from the group consisting of an amino group or a guanidino group, further wherein when said R16 is hydrogen, said R17 is a guanidino group or an amino group, and when said R16 is an amino group, said R17 is an amino group; R18 and R19 are independently selected from the group consisting of a hydrogen, a hydroxy, a lower alkyl group, a lower alkoxy group, and an aryl group; further wherein, said lower alkoxy group is selected from the group consisting of 1 to 6 carbon atoms, and said aryl group is selected from the group consisting of 6 to 10 carbon atoms.

In another aspect of the invention, the compound is selected from the group consisting of 2-guanidinobenzimidazole, 1,2-diaminobenzimidazole, 1,2-diaminobenzimidazole hydrochloride, 5-bromo-2-guanidinobenzimidazole, 5-methoxy-2-guanidinobenzimidazole, 5-methylbenzimidazole-1,2-diamine, 5-chlorobenzimidazole-1,2-diamine, and 2,5-diaminobenzimidazole.

In another aspect of the invention, the structural formula is structural formula IX:
R₂₀—CH—(NHR₂₁)—CO₂H IX
wherein, R20 is selected from the group consisting of a hydrogen, a lower alkyl group, a lower alkylthiol group, a carboxy group, an aminocarboxy group and an amino group; R21 is selected from the group consisting of a hydrogen and an acyl group; further wherein said lower alkyl group is selected from the group consisting of 1 to 6 carbon atoms and said acyl group is selected from the group consisting of 2 to 10 carbon atoms.

In another aspect of the invention, the compound is selected from the group is consisting of lysine, 2,3-diaminosuccinic acid, and cysteine.

In another aspect of the invention, the compound is a compound comprising the formula of said structural formula X: 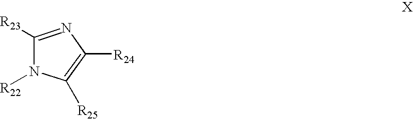
wherein R22 is selected from the group consisting of a hydrogen, an amino group, a mono-amino lower alkyl group, and a di-amino lower alkyl group; R23 is selected from the group consisting of a hydrogen, an amino group, a mono-amino lower alkyl group, and a di-amino lower alkyl group; R24 is selected from the group consisting of a hydrogen, a lower alkyl group, an aryl group and an acyl group; R25 is selected from the group consisting of a hydrogen, a lower alkyl group, an aryl group and an acyl group; further wherein, one of said R22 or R23 must be an amino group, or a mono- or di-amino lower alkyl group; said lower alkyl group is selected from the lower alkyl group consisting of 1 to 6 carbon atom; said mono- or di-amino alkyl groups are lower alkyl groups substituted by one or two amino groups; said aryl group is selected from the aryl group consisting of 6 to 10 carbon atoms; said acyl group is selected from the group consisting of a lower alkyl group, an aryl group, and a heteroaryl carboxylic acid containing 2 to 10 carbon atoms; and said lower alkoxy group is selected from the group consisting of 1 to 6 carbon atoms.

In another aspect of the invention, the compound is selected from the group consisting of 1,2-diamino-4-phenyl[1H]imidazole, 1,2-diaminoimidazole, 1-(2, 3-diaminopropyl)imidazole trihydrochloride, 4-(4-bromophenyl)imidazole-1,2-diamine, 4-(4-chlorophenyl)imidazole-1,2-diamine, 4-(4-hexylphenyl)imidazole-1,2-diamine, 4-(4-methoxyphenyl)imidazole-1,2-diamine, 4-phenyl-5-propylimidazole -1,2-diamine, 1,2-diamino-4-methylimidazole, 1,2-diamino-4,5-dimethylimidazole, and 1,2-diamino-4-methyl-5-acetylimidazole.

In another aspect of the invention, the structural formula is structural formula XI: 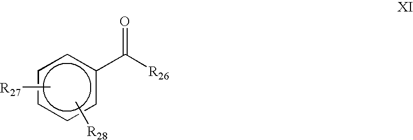
wherein R26 is selected from the group consisting of a hydroxy, a lower alkoxy group, an amino group, an amino lower alkoxy group, a mono-lower alkylamino lower alkoxy group, a di-lower alkylamino lower alkoxy group, a hydrazino group, and the formula NR29R30; R29 is selected from the group consisting of a hydrogen and a lower alkyl group; R30 is selected from the group consisting of an alkyl group of 1 to 20 carbon atoms,-an aryl group, a hydroxy lower alkyl group, a carboxy lower alkyl group, a cyclo lower alkyl group and a heterocyclic group containing 4 to 7 ring members and 1 to 3 heteroatoms; further wherein, said R29, R30, and nitrogen form a structure selected from the group consisting of a morpholino, a piperidinyl, and a piperazinyl; R27 is selected from the group consisting of 0 to 3 amino groups, 0 to 3 nitro groups, 0 to 1 hydrazino group, a hydrazinosulfonyl group, a hydroxyethylamino group, and an amidino group; R28 is selected from the group consisting of a hydrogen, a one-fluoro, a two-fluoro, a hydroxy, a lower alkoxy, a carboxy, a lower alkylamino, a di-lower alkylamino and a hydroxy lower alkylamino group; further wherein, when said R26 is a hydroxy or a lower alkoxy, then said R27 is a non-hydrogen substituent; further wherein, when R26 is hydrazino, there must be at least two non-hydrogen substituents on said formula XI's phenyl ring; when said R28 is hydrogen, said R30 is selected from the group consisting of an alkyl group of 1 to 20 carbon atoms, an aryl group, a hydroxy lower alkyl group, a carboxy lower alkyl group, a cyclo lower alkyl group, a heterocyclic group containing 4 to 7 ring members and 1 to 3 heteroatoms, an aminoimino group, a guanidyl group, an aminoguanidinyl group, and a diaminoguanidyl group; said lower alkyl group is selected from the group consisting of 1 to 6 carbon atoms; and said cycloalkyl group is selected from the group consisting of 4 to 7 carbon atoms.

In another aspect of the invention, the compound is selected from the group consisting of 4-(cyclohexylamino-carbonyl)-o-phenylene diamine hydrochloride, 3,4-diaminobenzhydrazide, 4-(n-butylamino-carbonyl)-o-phenylene -diamine dihydrochloride, 4-(ethylamino-carbonyl)-o-phenylene-diamine dihydrochloride, 4-carbamoyl-o-phenylene diamine hydrochloride, 4-(morpholino -carbonyl)-o-phenylene-diamine hydrochloride, 4-[(4-morpholino)hydrazino -carbonyl]-o-phenylenediamine, 4-(1-piperidinylamino-carbonyl)-o-phenylenediamine dihydrochloride, 2,4-diamino-3-hydroxybenzoic acid, 4,5-diamino-2-hydroxybenzoic acid, 3,4-diaminobenzamide, 3,4-diaminobenzhydrazide, 3,4-diamino-N,N-bis(1-methylethyl)benzamide, 3,4-diamino-N,N-diethylbenzamide, 3,4-diamino-N,N-dipropylbenzamide, 3,4-diamino-N-(2-furanylmethyl)benzamide, 3,4-diamino-N-(2-methylpropyl)benzamide, 3,4-diamino-N-(5-methyl-2-thiazolyl)benzamide, 3,4-diamino-N-(6-methoxy-2-benzothiazolyl)benzamide, 3,4-diamino-N-(6-methoxy-8-quinolinyl)benzamide, 3,4-diamino-N-(6-methyl-2-pyridinyl)benzamide, 3,4-diamino-N-(1H-benzimidazol-2-yl)benzamide, 3,4-diamino-N-(2-pyridinyl)benzamide, 3,4diamino-N-(2-thiazolyl)benzamide, 3,4-diamino-N-(4-pyridinyl)benzamide, 3,4-diamino-N-[9H-pyrido(3,4-b)indol-6-yl]benzamide, 3,4-diamino-N-butylbenzamide, 3,4-diamino-N-cyclohexylbenzamide, 3,4-diamino-N-cyclopentylbenzamide, 3,4-diamino-N-decylbenzamide, 3,4-diamino-N-dodecylbenzamide, 3,4-diamino-N-methylbenzamide, 3,4-diamino-N-octylbenzamide, 3,4-diamino-N-pentylbenzamide, 3,4-diamino-N-phenylbenzamide, 4-(diethylamino-carbonyl)-o-phenylene diamine, 4-(tert-butylamino-carbonyl)-o-phenylene diamine, 4-isobutylamino-carbonyl)-o-phenylene diamine, 4-(neopentylamino-carbonyl)-o-phenylene diamine, 4-(dipropylamino-carbonyl)-o-phenylene diamine, 4-(n-hexylamino-carbonyl)-o-phenylene diamine, 4-(n-decylamino-carbonyl) -o-phenylene diamine, 4-(n-dodecylamino-carbonyl)-o-phenylene diamine, 4-(1-hexadecylamino-carbonyl)-o-phenylene diamine, 4-(octadecylamino-carbonyl) -o-phenylene diamine,4-(hydroxylamino-carbonyl)-o-phenylene diamine, 4-(2-hydroxyethylamino-carbonyl)-o-phenylene, 4-[(2-hydroxyethylamino)ethylamino-carbonyl]-o-phenylene diamine, 4-[(2-hydroxyethyloxy)ethylamino-carbonyl]-o-phenylene diamine, 4-(6-hydroxyhexylamino-carbonyl)-o-phenylene diamine, 4-(3-ethoxypropylamino-carbonyl) -o-phenylene diamine, 4-(3-isopropoxypropylamino-carbonyl)-o-phenylene diamine, 4-(3-dimethylaminopropylamino-carbonyl)-o-phenylene diamine, 4-[4-(2-aminoethyl)morpholino-carbonyl]-o-phenylene diamine, 4-[4-(3-aminopropyl) morpholino-carbonyl]-o-phenylene diamine, 4-N-(3-aminopropyl)pyrrolidino-carbonyl]-o-phenylene diamine, 4-[3-(N-piperidino)propylamino-carbonyl]-o-phenylene diamine, 4-[3-(4-methylpiperazinyl)propylamino-carbonyl]-o-phenylene diamine, 4-(3-imidazoylpropylamino-carbonyl)-o-phenylene diamine, 4-(3-phenylpropylamino-carbonyl)-o-phenylenediamine, 4-[2-(N, N-diethylamino) ethylamino-carbonyl]-o-phenylene diamine, 4-(imidazolylamino-carbonyl)-o-phenylene diamine, 4-(pyrrolidinyl-carbonyl)-o-phenylene diamine, 4-(piperidino-carbonyl) -o-phenylene diamine, 4-(1-methylpiperazinyl-carbonyl)-o-phenylene diamine, 4-(2,6-dimethylmorpholino-carbonyl)-o-phenylenediamine, 4-(pyrrolidin-1-ylamino-carbonyl)-o-phenylene diamine, 4-(homopiperidin-1-ylamino-carbonyl)-o-phenylene diamine, 4-(4-methylpiperazine-1-ylamino-carbonyl)-o-phenylene diamine; 4-(1,2,4-triazol- 1-ylamino-carbonyl)-o-phenylene diamine, 4-(guanidinyl-carbonyl) -o-phenylene diamine, 4-(guanidinylamino-carbonyl)-o-phenylene diamine, 4-aminoguanidinylamino-carbonyl)-o-phenylene diamine, 4-(diaminoguanidinylamino-carbonyl) -o-phenylene diamine, 3,4-aminosalicylic acid 4-guanidinobenzoic acid, 3,4-diaminobenzohydroxamic acid, 3,4,5-triaminobenzoic acid, 2,3-diamino-5-fluoro-benzoic acid, and 3,4-diaminobenzoic acid.

In another aspect of the invention, the structural formula is structural formula XII: 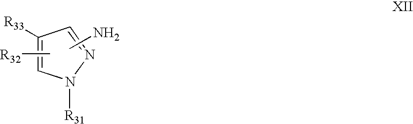
wherein R31 is selected from the group consisting of a hydrogen, a lower alkyl group and a hydroxy group; R32 is selected from the group consisting of a hydrogen, a hydroxy lower alkyl group, a lower alkoxy group, a lower alkyl group, and an aryl group; R33 is selected from the group consisting of a hydrogen and an amino group; said lower alkyl group is selected from the group consisting of 1 to 6 carbon atoms; said lower alkoxy group is selected from the group consisting of 1 to 6 carbon atoms; said hydroxy lower alkyl group is selected from the group consisting of primary, secondary and tertiary alcohol substituent patterns; said aryl group is selected from the group consisting of 6 to 10 carbon atoms; and a halo atom, wherein said halo atom is selected from the group consisting of a fluoro, a chloro, a bromo, and an iodo.

In another aspect of the invention, the compound is selected from the group consisting of 3,4-diaminopyrazole, 3,4-diamino-5-hydroxypyrazole, 3,4-diamino-5-methylpyrazole, 3,4-diamino-5-methoxypyrazole, 3,4-diamino-5-phenylpyrazole, 1-methyl-3-hydroxy-4,5-diaminopyrazole, 1-(2-hydroxyethyl)-3-hydroxy-4,5-diaminopyrazole, 1-(2-hydroxyethyl)-3-phenyl-4,5-diaminopyrazole, 1-(2-hydroxyethyl)-3-methyl-4,5-diaminopyrazole, 1-(2-hydroxyethyl)-4,5-diaminopyrazole, 1-(2-hydroxypropyl)-3-hydroxy-4,5-diaminopyrazole, 3-amino-5-hydroxypyrazole, and 1-(2-hydroxy-2-methylpropyl)-3-hydroxy-4,5-diaminopyrazole.

In another aspect of the invention, the structural formula is structural formula XIII: 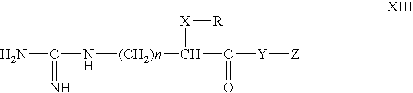
wherein n=1-6; X is selected from the group consisting of —NR1—, —S(O)—, —S(O)2—, and —O—, further wherein R1 is selected from the group consisting of H, linear chain alkyl group (C1-C6) and branched chain alkyl group (C1-C6); Y is selected from the group consisting of —N—, —NH—, and —O—; Z is selected from the group consisting of H, linear chain alkyl group (C1—C6), and branched chain alkyl group (C1-C6).

In another aspect of the invention, the structural formula is structural formula XIV: 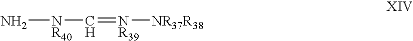
wherein R37 is selected from the group consisting of a lower alkyl group and a group of the formula NR41NR42; further wherein R41 and R42 together are selected from the group consisting of R41 is hydrogen and R42 is a lower alkyl group, R41 is hydrogen and R42 is a hydroxy (lower) alkyl group, and R41 and R42 together with said nitrogen atom form a heterocyclic group, further wherein said heterocyclic group contains 4 to 6 carbon atoms and 0 to 1 additional atoms selected from the group consisting of oxygen, nitrogen and sulfur; R38 is selected from the group consisting of a hydrogen and an amino group; R39 is selected from the group consisting of a hydrogen and an amino group; R40 is selected from the group consisting of a hydrogen and a lower alkyl group; further wherein at least one of said R38, R39, and R40 is other than hydrogen and one of said R37 and said R38 cannot be an amino group; said lower alkyl group is selected from the group consisting of 1 to 6 carbon atoms; said heterocyclic group formed by the NR41R42 group is a 4 to 7 membered ring containing 0 to 1 additional heteroatoms.

In another aspect of the invention, the compound is selected from the group consisting of 2-(2-hydroxy-2-methylpropyl)hydrazinecarboximidic hydrazide, N-(4-morpholino)hydrazinecarboximidamide, 1-methyl-N-(4-morpholino)hydrazinecarboximidamide, 1-methyl-N-(4-piperidino)hydrazinecarboximidamide, 1-(N-hexahydroazepino) hydrazinecarboximidamide, N,N-dimethylcarbonimidic dihydrazide, 1-methylcarbonimidic dihydrazide, 2-(2-hydroxy-2-methylpropyl) carbohydrazonic dihydrazide, and N-ethylcarbonimidic dihydrazide.

In another aspect of the invention, the structural formula is structural formula V: 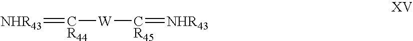
wherein R43 is selected from the group consisting of a pyridyl, a phenyl, and a carboxylic acid substituted phenyl group; wherein R46 is selected from the group lo consisting of a hydrogen, a lower alkyl group, and a water-solubilizing moiety; wherein W is selected from the group consisting of a carbon-carbon bond and an alkylene group of 1 to 3 carbon atoms; R44 is selected from the group consisting of a lower alkyl group, an aryl group, and a heteroaryl group; R45 is selected from the group consisting of a hydrogen, a lower alkyl group, an aryl group, and a heteroaryl group; said lower alkyl group is selected from the group consisting of 1 to 6 carbon atoms; said alkylene group is selected from the group consisting of a straight chain and a branched chain; said aryl group is selected from the group consisting of 6 to 10 carbon atoms; a halo atom is selected from the group consisting of a fluoro, a chloro, a bromo, and an iodo; said lower alkoxy group is selected from the group consisting of 1 to 6 carbon atoms; and said heteroaryl group is selected from the group consisting of 1 heteroatom and 2 heteroatoms.

In another aspect of the invention, the compound is selected from the group consisting of methylglyoxal bis-(2-hydrazino-benzoic acid)hydrazone, methylglyoxal bis-(dimethyl-2-hydrazinobenzoate)hydrazone, methylglyoxal bis-(phenylhydrazine) hydrazone, methyl glyoxal bis-(dimethyl-2-hydrazinobenzoate) hydrazone, methylglyoxal bis-(4-hydrazinobenzoic acid) hydrazone, methylglyoxal bis-(dimethyl4-hydrazinobenzoate)hydrazone, methylglyoxal bis-(2-pyridyl)hydrazone, methylglyoxal bis-(diethyleneglycol methylether-2-hydrazinobenzoate)hydrazone, methylglyoxal bis-[1-(2,3-dihydroxypropane)-2-hydrazinebenzoatehydrazone, methyl glyoxal bis-[1-(2-hydroxyethane)-2-hydrazinobenzoate]hydrazone, methylglyoxal bis-[(1-hydroxymethyl-l-acetoxy))-2-hydrazino-2-benzoate]hydrazone, methylglyoxal bis-[(4-nitrophenyl) -2-hydrazinobenzoate]hydrazone, methylglyoxal bis-[(4-methylpyridyl)-2-hydrazinobenzoate]hydrazone, methylglyoxal bis-(triethylene glycol 2-hydrazinobenzoate)hydrazone, and methylglyoxal bis-(2-hydroxyethylphosphate-2-hydrazinebenzoate)hydrazone.

In another aspect of the invention, the structural formula is structural formula XVI: 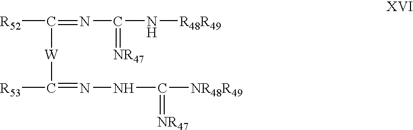
wherein R47 is selected from the group consisting of hydrogen and together with R48 an alkylene group of 2 to 3 carbon atoms; wherein said R48 is selected from the group consisting of hydrogen and alk-N-R5051, when said R47 is a hydrogen; further wherein, said alk is a straight or branched chain alkylene group of 1 to 8 carbon atoms, said R50 and R51 are independently each a lower alkyl group of 1 to 6 carbon atoms, or said R50 and said R5 1 together with said nitrogen atom form a group selected from the group consisting of a morpholino, a piperdinyl and a methylpiperazinyl; R49 is a hydrogen or said R49 is a hydroxyethyl when said R47 and said R48 are together an alkylene group of 2-3 carbon atoms; W is selected from the group consisting of a carbon-carbon bond, an alkylene group of 1 to 3 carbon atoms, a 1,2-, 1,3- or 1,4- phenylene group, a 2,3-naphthylene group, a 2,5-thiophenylene group, a 2,6-pyridylene group, an ethylene group, an ethenylene group, and a methylene group; R52 is selected from the group consisting of a lower alkyl group, an aryl group, and a heteroaryl group; R53 is selected from the group consisting of a hydrogen, a lower alkyl group, an aryl group, and a heteroaryl group; further wherein, when W is a carbon-carbon bond, R52 and R53 together can also be a 1,4-butylene group, or when W is a 1,2-, 1,3-, or 1,4-phenylene group, optionally substituted by one or two lower alkyl or amino groups, R52 and R53 are both hydrogen or a lower alkyl group; when W is an ethylene group, R52 and R53 together are an ethylene group; when W is a methylene group and R52 and R53 together are a group of the formula ═C (—CH3)-N-(H3C—) C═or-C-W-C-, then R52 and R53 together form a bicyclo-(3,3,1)-nonane or a bicyclo-3,3,1-octane group and R47 and R48 are together an alkylene group of 2-3 carbon atoms and R49 is hydrogen; said lower alkyl group is selected from the group consisting of 1 to 6 carbon atoms and said group may be optionally substituted by a halo hydroxy, an amino group or lower alkylamino group; said alkylene group is selected from the group consisting of straight and branched chain; said aryl group is selected from the group consisting of 6 to 10 carbon atoms; a halo atom, selected from the group consisting of a fluoro, a chloro, a bromo and an iodo; said lower alkoxy group is selecting from the group consisting of 1 to 6 carbon atoms, and said heteroaryl group is selected from the group consisting of 1 to 2 heteroatoms.

In another aspect of the invention, the compound is selected from the group consisting of methyl glyoxal bis(guanylhydrazone), methyl glyoxal bis(2-hydrazino-2-imidazoline-hydrazone), terephthaldicarboxaldehyde bis(2-hydrazino-2-imidazoline hydrazone), terephaldicarboxaldehyde bis(guanylhydrazone), phenylglyoxal bis(2-hydrazino-2-imidazoline hydrazone), furylglyoxal bis(2-hydrazino-2-imidazoline hydrazone), methyl glyoxal bis (1-(2-hydroxyethyl)-2-hydrazino-2-imidazoline hydrazone), methyl glyoxal bis (1-(2-hydroxyethyl)-2-hydrazino-1,4,5,6-tetrahydropyrimidine hydrazone), phenyl glyoxal bis (guanylhydrazone), phenyl glyoxal bis (1-(2-hydroxyethyl)-2-hydrazino-2-imidazoline hydrazone), furyl glyoxal bis (1-(2-hydroxyethyl)-2-hydrazino-2-imidazoline hydrazone), phenyl glyoxal bis (1- (2-hydroxyethyl)-2-hydrazino-1,4,5,6-tetrahydropyrimidine hydrazone), furyl glyoxal bis (1-(2-hydroxyethyl)-2-hydrazino -1,4,5,6-tetrahydropyrimidine hydrazone), 2,3-butanedione bis (2-hydrazino-2-imidazoline hydrazone), 1,4-cyclohexanedione bis(2-hydrazino-2-imidazoline hydrazone), o-phthalic dicarboxaldehyde bis(2-hyd carboximidamide hydrazone), furylglyoxal bis(guanyl hydrazone)dihydrochloride dihydrate, 2,3-pentanedione bis(2-tetrahydropyrimidine)hydrazone dihydrobromide, 1,2-cyclohexanedione bis(2-tetrahydropyrimidine)hydrazone dihydrobromide, 2,3-hexanedione bis(2-tetrahydropyrimidine)hydrazone dihydrobromide, 1,3-diacetyl bis(2-tetrahydropyrimidine)hydrazone dihydrobromide, 2,3-butanedione bis(2-tetrahydropyrimidine)hydrazone dihydrobromide, 2,6-diacetylpyridine-bis-(2-hydrazino-2-imidazoline hydrazone)dihydrobromide; 2,6-diacetylpyridine-bis-(guanyl hydrazone)dihydrochloride,2,6-pyridine dicarboxaldehyde-bis-(2-hydrazino-2-imidazoline hydrazone)dihydrobromide trihydrate), 2,6-pyridine dicarboxaldehyde -bis (guanyl hydrazone)dihydrochloride; 1,4-diacetyl benzene-bis-(2-hydrazino-2-imidazoline hydrazone)dihydrobromide dihydrate, 1,3-diacetyl benzene-bis-(2-hydrazin o-2-imidazoline)hydrazone dihydrobromide, 11,3-diacetyl benzene-bis (guanyl)-hydrazone dihydrochloride, isophthalaldehyde-bis-(2-hydrazino-2-imidazoline) hydrazone dihydrobromide, isophthalaldehyde-bis-(guanyl)hydrazone dihydrochloride, 2,6-diacetylaniline bis-(guanyl)hydrazone dihydrochloride, 2,6-diacetyl aniline bis-(2-hydrazino-2-imidazoline)hydrazone dihydrobromide, 2,5-diacetylthiophene bis(guanyl)hydrazone dihydrochloride, 2,5-diacetylthiophene bis-(2-hydrazino-2-imidazoline)hydrazone dihydrobromide, 1,4-cyclohexanedione bis(2-tetrahydropyrimidine)hydrazone dihydrobromide, 3,4-hexanedione bis(2-tetrahydropyrimidine)hydrazone dihydrobromide, methylglyoxal-bis-(4-amino-3-hydrazino-1,2,4-triazole)hydrazone dihydrochloride, methylglyoxal-bis-(4-amino-3-hydrazino-5-methyl- 1,2,4-triazole)hydrazone dihydrochloride, 2,3-pentanedione-bis-(2-hydrazino-3-imidazoline)hydrazone dihydrobromide, 2,3-hexanedione-bis-(2-hydrazino-2-imidazoline)hydrazone dihydrobromide, 3-ethyl-2,4-pentane dione-bis-(2-hydrazino-2-imidazoline)hydrazone dihydrobromide, methylglyoxal-bis-(4-amino-3-hydrazino-5-ethyl-1,2,4-triazole)hydrazone dihydrochloride, methylglyoxal-bis-(4-amino-3-hydrazino-5-isopropyl-1,2,4-triazole)hydrazone dihydrochloride, methyl glyoxal-bis-(4-amino-3-hydrazino-5-cyclopropyl-1,2,4-triazole)hydrazone dihydrochlorimethylglyoxal-bis-(4-amino-3-hydrazino-5-cyclobutyl-12,4-triazole) hydrazone dihydrochloride, 1,3-cyclohexanedione-bis-(2-hydrazino-2-imidazoline) hydrazone dihydrobromide, 6-dimethyl pyridine bis(guanyl)hydrazone dihydrochloride, 3,5-diacetyl-1,4-dihydro-2,6-dimethylpyridine bis-(2-hydrazino-2-imidazoline hydrazone dihydrobromide, bicyclo-(3,3,1)nonane-3,7-dione bis-(2-hydrazino-2-imidazoline)hydrazone dihydrobromide, and cis-bicyclo-(3,3, 1 )octane-3,7-dione bis-(2-hydrazino-2-imidazoline)hydrazone dihydrobromide.

In another aspect of the invention, the structural formula is structural formula XVII: 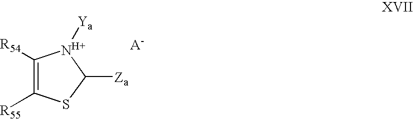
wherein R54 is selected from the group consisting of a hydrogen, a hydroxy (lower) alkyl group, a lower acyloxy (lower) alkyl group, and a lower alkyl group; R55 is selected from the group consisting of a hydrogen, a hydroxy (lower) alkyl group, a lower acyloxy (lower) alk-yl group, and a lower alkyl group; further wherein R54 and R55 together with their ring carbons may be an aromatic fused ring; Za is hydrogen or an amino group; Ya is selected from the group consisting of a hydrogen, a group of the formula -CH2C(═O)—R56and a group of the formula —CHR′, further wherein, when said Ya is a group of said formula —CH2C(═O)—R56said R is selected from the group consisting of a lower alkyl group, an alkoxy group, a hydroxy, an amino group, and an aryl group; wherein when said Ya is a group of said formula —CHR′, said R′ is selected from the group consisting of a hydrogen, a lower alkyl group, a lower alkynyl group, and an aryl group; wherein A is selected from the group consisting of a halide, a tosylate, a methanesulfonate, and a mesitylenesulfonate ion; said lower alkyl group is selected from the group consisting of 1-6 carbon atoms; said lower alkynyl group is selected from the group consisting of 2 to 6 carbon atoms; said lower alkoxy group is selected from the group consisting of 1 to 6 carbon atoms; said lower acyloxy (lower) alkyl group contains an acyloxy portion and a lower alkyl portion, further wherein said acyloxy portion is selected from the group consisting of 2 to 6 carbon atoms and said lower alkyl portion is selected from the group consisting of 1 to 6 carbon atoms; said aryl group is selected from the group consisting of 6 to 10 carbon atoms; and a halo atom of formula XVII is selected from the group consisting of a fluoro, a chloro, a bromo, and an iodo.

In another aspect of the invention, the compound is selected from the group consisting of 3-aminothiazolium mesitylenesulfonate, 3-amino-4,5-dimethylaminothiazolium mesitylenesulfonate, 2,3-diaminothiazolinium mesitylenesulfonate, 3-(2-methoxy-2-oxoethyl)-thiazolium bromide, 3-(2-methoxy-2-oxoethyl)-4,5-dimethylthiazolium bromide, 3-(2-methoxy-2-oxoethyl)-4-methylthiazolium bromide, 3-(2-phenyl-2-oxoethyl)-4-methylthizolium bromide, 3-(2-phenyl-2-oxoethyl)-4,5-dimethylthiazolium bromide, 3-amino-4-methylthiazolium mesitylenesulfonate, 3-(2-methoxy-2-oxoethyl)-5-methylthiazolium bromide, 3-(3-(2-phenyl-2-oxoethyl)-5-methylthiazolium bromide, 3-[2-(4′-bromophenyl)-2-oxoethyl] thiazolium bromide, 3- [2-(4′-bromophenyl)-2-oxoethyl]-4-methylthiazolium bromide, 3-[2-(4′-bromophenyl)-2-oxoethyl]-5-methylthiazolium bromide, 3-[2-(4′bromophenyl)-2-oxoethyl]-4,5-dimethylthiazolium bromide, 3-(2-methoxy-2-oxoethyl)-4-methyl-5-(2-hydroxyethyl) thiazolium bromide, 3-(2-phenyl-2-oxoethyl)-4-methyl-5-(2-hydroxyethyl) thiazolium bromide, 3-[2-(4′-bromophenyl)-2-oxoethyl]methyl-5-(2-hydroxyethyl) thiazolium bromide, 3,4-dimethyl-5-(2-hydroxyethyl) thiazolium iodide, 3-ethyl-5-(2-hydroxyethyl)4-methylthiazolium bromide, 3-benzyl-5-(2-hydroxyethyl)-4-methylthiazolium chloride, 3-(2-methoxy-2-oxoethyl)benzothiazolium bromide, 3-(2-phenyl-2-oxoethyl)benzothiazolium bromide, 3-[2-(4′bromophenyl)-2-oxoethyl] benzothiazolium bromide, 3-(carboxymethyl) benzothiazolium bromide, 2,3-(diamino) benzothiazolium mesitylenesulfonate, 3-(2-amino-2-oxoethyl) thiazolium bromide, 3-(2-amino-2-oxoethyl)-4-methylthiazolium bromide, 3-(2-amino-2-oxoethyl)-5-methylthiazolium bromide, 3-(2-amino-2-oxoethyl) 4,5-dimethylthiazolium bromide, 3-(2-amino-2-oxoethyl)benzothiazolium bromide, 3-(2-amino-2- oxoethyl) 4-methyl-5-(2-hydroxyethyl)thiazolium bromide, 3-amino-5-(2-hydroxyethyl)-4-methylthiazolium mesitylenesulfonate, 3-(2-methyl-2-oxoethyl)thiazolium chloride, 3-amino-4-methyl -5-(2-acetoxyethyl)thiazolium mesitylenesulfonate, 3-(2-phenyl-2-oxoethyl)thiazolium bromide, 3-(2-methoxy-2-oxoethyl)-4-methyl-5-(2-acetoxyethyl) thiazoliumbromide, 3-(2-amino-2-oxoethyl)-4-methyl-5- (2-acetoxyethyl)thiazolium bromide, 2-amino-3-(2-methoxy-2-oxoethyl) thiazolium bromide, 2-amino-3-(2-methoxy-2-oxoethyl) benzothiazolium bromide, 2-amino-3-(2-amino-2-oxoethyl)thiazolium bromide, 2-amino-3-(2-amino-2-oxoethyl)benzothiazolium bromide, 3-[2-(4′-methoxyphenyl)-2-oxoethyl]-thiazolium bromide, 3-[2-(2′, 4′-dimethoxyphenyl)-2-oxoethyl]-thiazolinium bromide, 3-[2-(4′-fluorophenyl)-2-oxoethyl]-thiazolinium bromide, 3-[2-(2′, 4′-difluorophenyl)-2-oxoethyl]-thiazolinium bromide, 3-[2-(4′-diethylaminophenyl)-2-oxoethyl]-thiazolinium bromide, 3-propargyl-thiazolinium bromide, 3-propargyl-4-methylthiazolinium bromide, 3-propargyl-5-methylthiazolinium bromide, 3-propargyl-4,5-dimethylthiazolinium bromide, and 3-propargyl-4-methyl-5-(2-hydroxyethyl)-thiazolinium bromide.

In another aspect of the invention, the structural formula is structural formula XVIII: 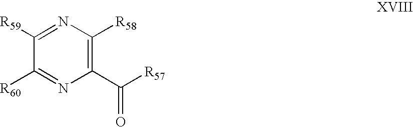
wherein, R57 is selected from the group consisting of a hydroxy, a NHCONCR6IR62and a N═C(NR61R62)2; R61 and R62 are each independently selected from the group consisting of a hydrogen, a 1 to 10 carbon atom straight chain alkyl, a 1 to 10 carbon atom branched chain alkyl, an aryl 1 to 4 carbon atom alkyl, a mono-substituted aryl 1 to 4 carbon alkyl, and a di-substituted aryl 1 to 4 carbon atom alkyl, wherein said substituents are selected from the group consisting of a fluoro, a chloro, a bromo, an iodo, a 1 to 10 carbon atom alkyl straight chain, and a 1 to 10 carbon atom alkyl branched chain; wherein R58 is selected from the group consisting of a hydrogen, an amino, a mono-substituted amino and a di-substituted amino, and R59 is selected from the group consisting of a hydrogen, an amino, a mono-substituted amino and a di-substituted amino; further wherein, when R58 and R59 are not both amino or substituted amino, the substituents are selected from the group consisting of a 1 to 10 carbon atom straight chain alkyl, a 1 to 10 carbon atom branched chain alkyl, and a 3 to 8 carbon atom cycloalkyl; and wherein R60 is selected from the group consisting of a hydrogen, a trifluoromethyl, a fluoro, a chloro, a bromo, and an iodo.

The present invention also features a method of treating a mammal having a disease selected from the group consisting of scleroderma, keloids, and scarring, wherein the mammal is in need of such treatment, comprising administering to the mammal an effective amount of a composition comprising at least one compound capable of disrupting a crosslinkage between crosslinked proteins. In one aspect, the compound is selected from the group consisting of compounds of the formula XXV: 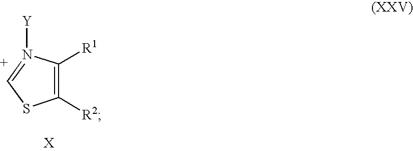
wherein R.sup.1 and R.sup.2 are independently selected from the group consisting of hydrogen and an alkyl group, which can be substituted by a hydroxy group; Y is a group of the formula —CH.sub.2 C(═O)R wherein R is a heterocyclic group other than alkylenedioxyaryl containing 4-10 ring members and 1-3 heteroatoms selected from the group consisting of oxygen, nitrogen and sulfur, the heterocyclic group can be substituted by one or more substituents selected from the group consisting of alkyl, oxo, alkoxycarbonylalkyl, aryl, and aralkyl groups; and said one or more substituents can be substituted by one or more alkyl or alkoxy groups; or group of the formula —CH.sub.2 C(.dbd.O)—NHR′ wherein R′ is a heterocyclic group other than alkylenedioxyaryl containing 4-10 ring members and 1-3 heteroatoms selected from the group consisting of oxygen, nitrogen, and sulfur, the heterocyclic group can be substituted by one or more alkoxycarbonylalkyl groups; and X is a pharmaceutically acceptable ion; and a carrier therefor.

The present invention also features a method of treating a mammal having a disease selected from the group consisting of scleroderma, keloids, and scarring, wherein the mammal is in need of such treatment, the method comprising administering to the mammal an effective amount of a composition comprising at least one compound capable of preventing protein crosslinkage. In another embodiment, the invention features a method of treating a mammal comprising administering to said mammal an effective amount of a composition comprising: at least one compound capable of preventing protein crosslinkage and at least one compound capable of disrupting a crosslinkage between crosslinked proteins.

In one embodiment, the invention features a method of preventing the crosslinking of collagen in a patient in need thereof, the method comprising administering to the patient a composition comprising a compound that inactivates 3DG. In one aspect, the compound inhibits the formation of 3DG. In another aspect, the compound is selected from the group consisting of compounds having structural formula I: 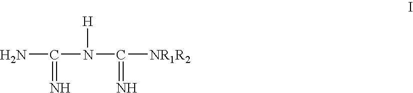
wherein R1 and R2 are independently selected from the group consisting of a hydrogen, a lower alkyl, a lower alkoxy and an aryl group; or wherein said R1 and said R2 together with a nitrogen atom form a heterocyclic ring containing from 1 to 2 heteroatoms and 2 to 6 carbon atoms, the second of said heteroatoms comprising nitrogen, oxygen, or sulfur; further wherein said lower alkyl group is selected from the group consisting of 1 to 6 carbon atoms; wherein said lower alkoxy group is selected from the group consisting of 1 to 6 carbon atoms; and wherein said aryl group comprises substituted and unsubstituted phenyl and pyridyl groups.

In another aspect of the invention, the compound is selected from the group consisting of meglumine, sorbitollysine, mannitollysine and galactitollysine. In another aspect of the invention, a patient has at least one disease selected from the group consisting of scleroderma, keloids and scarring.

The present invention also features a method of inhibiting fructosamine kinase in a mammal, the method comprising administering to the mammal a composition comprising a copper-containing compound. In one aspect, the copper-containing compound is selected from the group consisting of a copper-salicylic acid conjugate, a copper-peptide conjugate, a copper-amino acid conjugate, and a copper salt. In another aspect, the copper-containing compound is selected from the group consisting of a copper-lysine conjugate and a copper-arginine conjugate. In one embodiment of the invention, the mammal has a disease associated with at least one diabetic complication. In an aspect, the diabetic complication is selected from the group consisting of retinopathy, neuropathy, cardiovascular disease, dementia, and nephropathy.

In an embodiment, the invention features a method of increasing the production of collagen in a mammal by administering to the mammal a composition that inhibits the Amadorase pathway, wherein the composition comprises a copper-containing compound, thereby increasing the production of collagen in the mammal. In one aspect, the copper-containing compound inhibits fructoseamine kinase. In another aspect, the collagen is Type I collagen. In yet another aspect, the collagen is Type III collagen. In still another aspect, the collagen comprises Type I and Type III collagens.

In an embodiment, the invention features a method of increasing the level of mRNA for collagen in a mammal, the method comprising administering to the mammal a composition that inhibits the Amadorase pathway, the composition comprising a copper-containing compound, thereby increasing the level of mRNA collagen in the mammal. In another embodiment, the invention features a method of decreasing desmosine levels in a mammal, the method comprising administering to the mammal a composition comprising an inhibitor of the Amadorase pathway, wherein the inhibitor is a copper-containing compound. In yet another embodiment, the invention features a method of stabilizing desmosine levels in a mammal, the method comprising administering to the mammal a composition comprising an inhibitor of the Amadorase pathway, wherein the inhibitor is a copper-containing compound.

The invention also features a method of decreasing the level of mRNA for collagen in a mammal by increasing the flux through the Amadori pathway in the mammal, the method comprising administering to the mammal a composition comprising at least one copper chelator. In one aspect, the compound is selected from the group consisting of triethylenetetramine dihydrochloride (triene), penicillamine, sar, diamsar, ethylenediamine tetraacetic acid, o-phenanthroline, and histidine.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments, which are presently preferred. It should be understood, however, that the invention is not limited to the precise arrangements and instrumentalities shown.

FIG. 1 is a schematic diagram depicting the initial step involved in the multi-step reaction leading to crosslinking of proteins.

FIG. 2 is a schematic diagram, which illustrates the reactions involved in the lysine recovery pathway. Fructoselysine (FL) is phosphorylated by a fructosamine kinase such as Amadorase to form fructoselysine 3-phosphate (FL3P). FL3P spontaneously decomposes into lysine, Pi, and 3DG (Brown et al., U.S. Pat. No. 6,004,958).

FIG. 3 is a graph representing a urinary profile showing the variation over time of 3DF, 3DG and FL from a single individual fed 2 grams of FL and followed for 24 hours.

FIG. 4 is a graph representing 3DF excretion in urine over time from seven volunteers fed 2 grams of fructoselysine.

FIG. 5 graphically compares 3DF and N-acetyl-β-glucosaminidase (NAG) levels in control animals and an experimental group maintained on feed containing 0.3% glycated protein (Brown et al., U.S. Pat. No. 6,004,958)

FIG. 6 is a graph that demonstrates the linear relationship between 3DF and 3DG levels in urine of rats fed either a control diet or a diet enriched in glycated protein (Brown et al., U.S. Pat. No. 6,004,958).

FIG. 7, comprising FIG. 7A and FIG. 7B, graphically depicts fasting levels of urinary 3DG in normal subjects and in diabetic patients, plotted against the fasting level of 3DF.

FIG. 8, comprising FIG. 8A and FIG. 8B, depicts images of photomicrographs illustrating the effects of a diet containing high levels of glycated protein on the kidney. Periodic acid and Schiff (PAS) stained kidney sections were prepared from a rat fed a diet enriched in mildly glycated protein (FIG. 8A) and a rat fed a normal diet (FIG. 8B). In this experiment, non-diabetic rats were fed a diet containing 3% glycated protein for 8 months. This diet substantially elevated levels of FL and its metabolites (>3-fold in the kidney). FIG. 8A is an image of a photomicrograph of a glomerulus from a rat fed the glycated diet for 8 months. The glomerulus shows segmental sclerosis of the glomerular tuft with adhesion of the sclerotic area to Bowman's capsule (lower left). There is also tubular metaplasia of the parietal epithelia from approximately 9 to 3 o'clock. These sclerotic and metaplastic changes are reminiscent of the pathologies observed in diabetic kidney disease. FIG. 8B is an image from a rat on the control diet for 8 months, comprising a histologically normal glomerulus.

FIG. 9 is a graphic comparison of 3DG and 3DF levels in glomerular and tubular fractions from rat kidneys after FL feeding.

FIG. 10 is an image depicting the nucleic acid sequence (SEQ ID NO: 1) of human Amadorase (fructosamine-3-kinase), NCBI accession number NM_{—}022158. The accession number for the human gene on chromosome 17 is NT_{—}010663.

FIG. 11 is an image depicting the amino acid sequence (SEQ ID NO:2) of human Amadorase (fructosamine-3-kinase), NCBI accession number NP_{—}071441.

FIG. 12 is an image of a polyacrylamide gel demonstrating the effects of 3DG on collagen crosslinking and the inhibition of 3DG induced crosslinking by arginine. Collagen type I was treated with 3DG in the-presence or absence of arginine. The samples were subjected to cyanogen bromide (CNBr) digestion, electrophoresed on a 16.5% SDS Tris-tricine gel, and then the gels were processed using silver stain techniques to visualize the proteins. Lane 1 contains molecular weight marker standards. Lanes 2 and 5 contain 10 and 20 μl of the collagen mixture following CNBr digestion. Lanes 3 and 6 contain the collagen mixture treated with 3DG and then digested with CNBr, and loaded at 10 and 20 μl, respectively. Lanes 4 and 7 contain the mixture of collagen incubated with 5 mM 3DG and 10 mM arginine and then digested with CNBr, and loaded at 10 and 20 μl, respectively.

FIG. 13 is an image of an agarose gel demonstrating that the mRNA for Amadorase/fructosamine kinase is present in human skin. RT-PCR was utilized and published Amadorase sequences were used as the basis for preparing templates for PCR. Based on the primers used (see Examples) for the PCR reaction, the presence of a 519 bp fragment in the gel indicates the presence of Amadorase mRNA. Expression of Amadorase, as based on the presence of Amadorase mRNA indicated by a 519 bp fragment, was found in the kidney (lane 1) and in the skin (lane 3). No 519 bp fragments were found in the control lanes, which contained primer but no template (lanes 2 and 4). Lane 5 contained DNA molecular weight markers.

FIG. 14 is a graphic illustration of the effects of DYN 12 (3-O-methylsorbitollysine) treatment on skin elasticity. Diabetic or normal rats were treated with DYN 12 (50 mg/kg daily) or saline for eight weeks and then subjected to skin elasticity tests. The four groups used included diabetic controls (saline injection; solid black bar), diabetics treated with DYN 12 (open bar), normal animal controls (saline injections; stippled bar), and normal animals treated with DYN 12 (cross-hatched bar). Data are expressed in kilopascals (kPA).

FIG. 15 is graphic illustration of the effects of DYN 12 (3-O -methylsorbitollysine)treatment on skin elasticity. Diabetic or normal rats were treated with DYN 12 (50 mg/kg daily) or saline for eight weeks and then subjected to skin elasticity tests. The four groups used included diabetic controls (saline injection; solid black bar), diabetics treated with DYN 12 (open bar), normal animal controls (saline injections; stippled bar), and normal animals treated with DYN 12 (cross-hatched bar). Data are expressed in kilopascals (kPA) and are shown as averages of the results obtained with each particular group of test subjects. Measurements were taken on the hind leg of the test subjects and were taken on an alert animal restrained by a technician.

FIG. 16 is a schematic illustration of a novel metabolic pathway in the kidney. The formation of 3DG in the kidney occurs using either endogenous glycated protein or glycated protein derived from dietary sources. By way of the endogenous pathway, the chemical combination of glucose and lysine leads to glycated protein. Alternatively, glycated protein may also be obtained from dietary sources. Catabolism of glycated proteins results in the production of fructoselysine, which is subsequently acted upon by Amadorase. Amadorase, a fructosamine-3-kinase, is part of both pathways. Amadorase phosphorylates fructoselysine to form fructoselysine-3-phosphate, which may then be converted to 3-deoxyglucosone (3DG), producing byproducts of lysine and inorganic phosphate (A very small amount of fructoselysine (<5% total fructoselysine) may be converted to 3DG by way of a non-enzymatic pathway). 3DG may then be detoxified by conversion to 3-deoxyfructose (3DF) or it may go on to produce reactive oxygen species (ROS) and advanced glycation end products (AGEs). As shown in FIG. 16, DYN 12 (3-O -methylsorbitollysine) inhibits the action of Amadorase on fructoselysine, and DYN 100 (arginine) inhibits the 3DG-mediated production of ROS and AGEs.

FIG. 17 is a schematic illustration of the disease states affected by reactive oxygen species (ROS). 3DG may produce ROS directly, or it may produce advanced glycation end products, which go on to form ROS. The ROS are then responsible for advancing various disease states as shown in the figure.

FIG. 18 is a schematic illustration of both adducts formation and inhibitions of adduct formation according to embodiments of the present invention. 3DG can form an adduct with a primary amino group on a protein. Protein-3DG adduct formation creates a Schiff base, the equilibrium of which is depicted in FIG. 18. The protein-3DG Schiff base adduct may go on to form a crosslinked protein, by formation of a second protein-3DG adduct by way of the 3DG molecule involved in the first protein-3DG Schiff base adduct described above, thereby forming a “3DG bridge” between two primary amino groups of a single protein (pathway “A”). Alternatively, such crosslinking may occur between two primary amino groups of separate proteins, forming a “3DG bridge” between two primary amino groups of two separate proteins, resulting in a crosslinked pair of protein molecules. The first protein-3DG Schiff base adducts may be prevented from going on to form such crosslinked proteins as depicted in pathway “A.” For example, such protein crosslinking may be inhibited by nucleophilic agents such as glutathione or penicillamine, as illustrated in FIG. 18 by pathway “B.” Such nucleophilic agents react with the 3DG carbon atom responsible for forming the second Schiff base, preventing that carbon atom from forming a Schiff base protein-3DG adduct and thereby preventing crosslinking of the protein.

FIG. 19 is a Northern blot with samples probed for Col 1A1 and GAPDH RNAs.

FIG. 20 is a graphic illustration of the effect of copper on the activity of Amadorase. The data are plotted as percent amadorase activity (y-axis) as a condition of copper sulfate concentration (x-axis). No copper added is 100% activity. As copper concentration increases, Amadorase activity is inhibited.

FIG. 21 is a plot of the effect of fructose lysine on collagen production in human dermal fibroblasts. Fibroblasts were treated with fructoselysine or magnesium ascorbate (As-PM) for 72 hr. Each bar represents the mean +SD of type I collagen concentration, and the line graph represents the mean of number of cells n=3). *P<0.05, ***P<0.001 vs control (Dunnett multiple comparison test).

FIG. 22 is a plot of DYN-12 on type I collagen production in human dermal fibroblasts. Fibroblasts were treated with DYN-12 or magnesium ascorbate (As-PM) for 72 hr. Each bar represents the mean±SD of type I collagen concentration, and line graph represents the mean of number of cells n=3). *P<0.05, ***P<0.001 vs control (Dunnett multiple comparison test).

### DETAILED DESCRIPTION OF THE INVENTION

The present invention, as described for the first time in the disclosure provided herein, is based on the surprising discovery that altering the flux through the Amadorase pathway results in changes in mRNA for collagen and the formation of desmosines, the essential components of elastin.

The invention therefore encompasses compositions and methods to decrease levels of mRNA for collagen by increasing the flux through the Amadorase pathway, which compositions and methods include administering compounds to a mammal that act as substrates to FL3K, upregulate FL3K, and generate free lysine.

The invention further encompasses treatment of diseases associated with excessive production of mRNA for collagen, by the administration of compounds that increase the flux through the Amadorase pathway and thereby decrease the levels of mRNA for collagen. Diseases associated with excessive levels of collagen type I include scleroderma, endomyocardial fibrosis, ARDS and lung fibrosis. The invention also encompasses the removal of 3DG produced by the increased flux through the Amadorase pathway, to protect from the toxic effects of 3DG.

The invention further encompasses treatment of diseases associated with decreased or low levels of mRNA for collagens. These diseases include aging, especially in skin and arteries and myopia, with regard to type I collagen, osteoarthritis and intervertebral disc disease with regard to type II collagen. The invention therefore encompasses compositions and methods to increase levels of mRNA for collagens by decreasing the flux through the Amadorase pathway, which compositions and methods include administering compounds to a mammal that act as substrates for FL3K that do not result in the production of 3DG and/or free lysine, compounds that inhibit FL3K and otherwise decrease the flux through the Amadorase pathway.

The invention encompasses compositions and methods to enhance success of collagen implants comprising the addition of compounds that increase levels of mRNA for collagens by decreasing the flux through the Amadorase pathway, which compositions and methods include administering said compounds to a mammal that has received a collagen implant or integrating said compounds into an implant prior to insertion into a mammal. The compounds encompassed in the invention include substrates for FL3K that do not result in the production of 3DG and/or free lysine, compounds that inhibit FL3K and compounds that otherwise decrease the flux through the Amadorase pathway.

The invention further includes the discovery that the levels of desmosines, in diabetes, are elevated, and that these levels can be reduced by methods and compounds that affect the Amadorase Pathway. The invention therefore encompasses compositions and methods to inhibit the flux through the enzyme fructoseamine 3 kinase, inhibiting the enzyme fructoseamine 3 kinase and inhibiting the formation of 3DG, as well as inactivating 3DG. Compounds that inhibit the enzyme and compounds that inactivate 3DG are set forth in detail elsewhere herein, and are referenced, in part, International Patent Application number of PCT/US03/12003 (Publication Number WO 03/089601) and in U.S. Pat. No. 6,006,958incorporated herein by reference.

The invention further encompasses compositions and methods to inhibit 3DG formation or to remove 3DG from organs containing elastin, as well as compositions and methods to increase the rate of detoxification and removal of 3DG from organs containing elastin.

The invention is further based on the concept that development of inelastic aged skin and inelastic elastin containing organs can be prevented and reversed by compositons and methods that inhibit the formation of desmosines by inhibiting the flux through the enzyme fructoseamine 3 kinase, inhibiting the enzyme fructoseamine 3 kinase and inhibiting the formation of 3DG, as well as inactivating 3DG. In elastin containing organs include the extracellular matrix that forms the internal structure of the body and its organs, and more specifically skin, lungs, ligament, blood vessels, and elastic cartilage.

The invention also encompasses methods and compositions to prevent and treat certain elastin related disease. Elastin related diseases include atherosclerosis, Buscke-Oljlendorff syndrome, cutis laxa, emphysema, Marfan syndrome, Menkes syndrome, pseudoxanthoma elasticum, supravalvular aortic stenosis and Williams syndrome.

Therefore the invention encompasses methods and compositions to inhibit the increased production of desmosines in elastin containing organs and methods and compositions to remove 3DG from said elastin containing organs. The invention also encompasses compositions of copper and copper containing compounds that inhibit the enzyme fructosamine 3 phosphate kinase.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “accumulation of 3DG” or “accumulation of alpha-dicarbonyl sugars” as used herein refers to a detectable increase in the level of 3DG and/or alpha-dicarbonyl sugar overtime.

“Alpha-dicarbonyl sugar,” as used herein, refers to a family of compounds, including 3-deoxyglucosone, glyoxal, methyl glyoxal and glucosone. “Alpha-dicarbonyl sugar associated parameter of wrinkling, aging, disease or disorder of the skin,” as used herein, refers to the biological markers described herein; including 3DG levels, 3DF levels, fructosamine kinase levels, protein crosslinking, and other markers or parameters associated with alpha-dicarbonyl sugar associated wrinkling, aging, diseases or disorders of the skin.

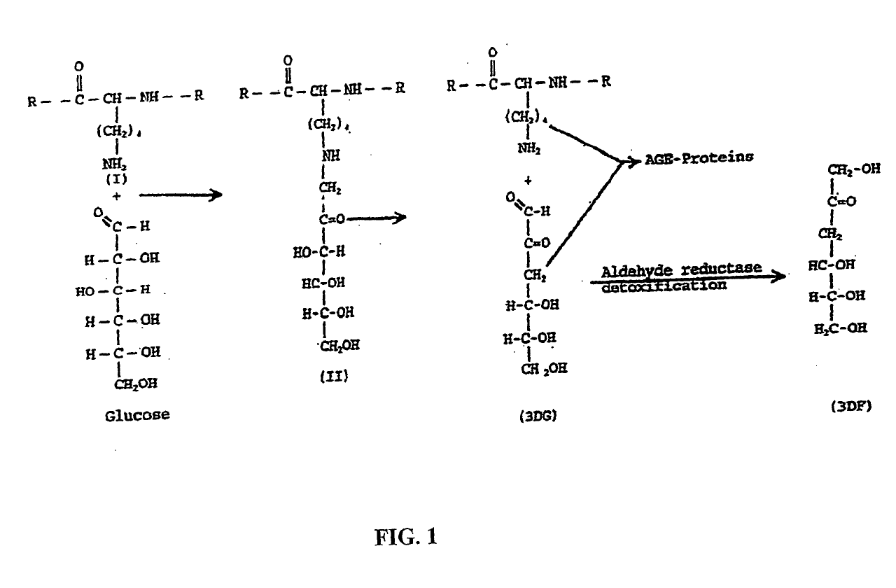

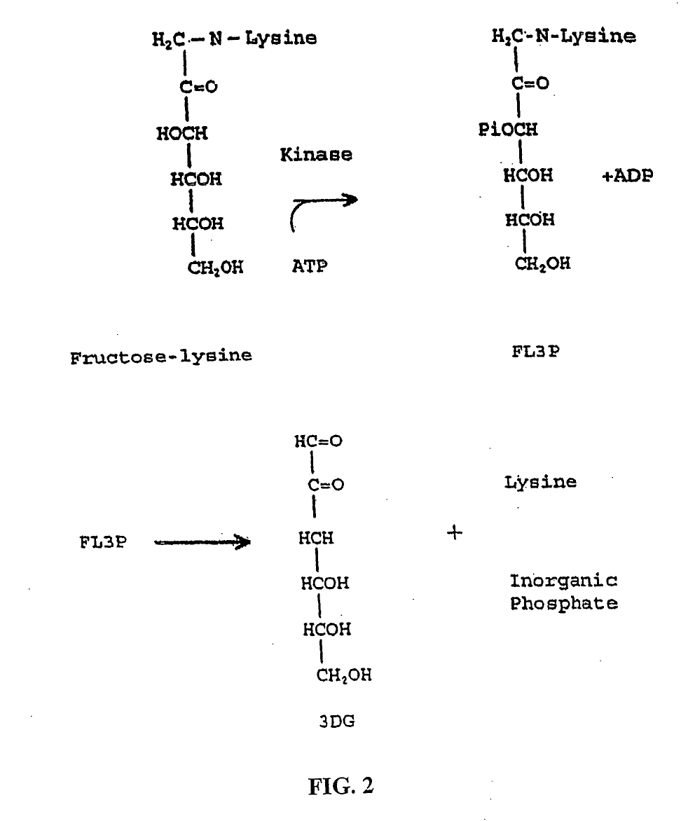

“3-Deoxyglucosone” or “3DG,” as used herein, refers to the 1,2-dicarbonyl-3-deoxysugar (also known as 3-deoxyhexulosone), which can be formed via an enzymatic pathway or can be formed via a nonenzymatic pathway. For purposes of the present description, the term 3-deoxyglucosone is an alpha-dicarbonyl sugar which can be formed by pathways including the nonenzymatic pathway described in FIG. 1 and the enzymatic pathway resulting in breakdown of FL3P described in FIG. 2. Another source of 3DG is diet. 3DG is a member of the alpha-dicarbonyl sugar family, also known as 2-oxoaldehydes.

A “3DG associated” or “3DG related” disease or disorder as used herein, refers to a disease, condition, or disorder which is caused by, indicated by or associated with 3DG, including defects related to enhanced synthesis, production, formation, and accumulation of 3DG, as well as those caused by medicated by or associated with decreased levels of degradation, detoxification, binding, and clearance of 3DG.

“A 3DG inhibiting amount” or an “alpha-dicarbonyl inhibiting amount” of a compound refers to that amount of compound which is sufficient to inhibit the function or process of interest, such as synthesis, formation accumulation and/or function of 3DG or another alpha-dicarbonyl sugar.

The term “3DG protein/peptide adducts” refers to covalent bonds formed between 3DG and amino acid residues on a protein or peptide.

“3-O-methyl sorbitollysine (3-0-Me-sorbitollysine),” is an inhibitor of fructosamine kinases, as described herein. It is used interchangeably with the term “DYN 12”.

As used herein, “alleviating a disease or disorder symptom,” means reducing the severity of the symptom.

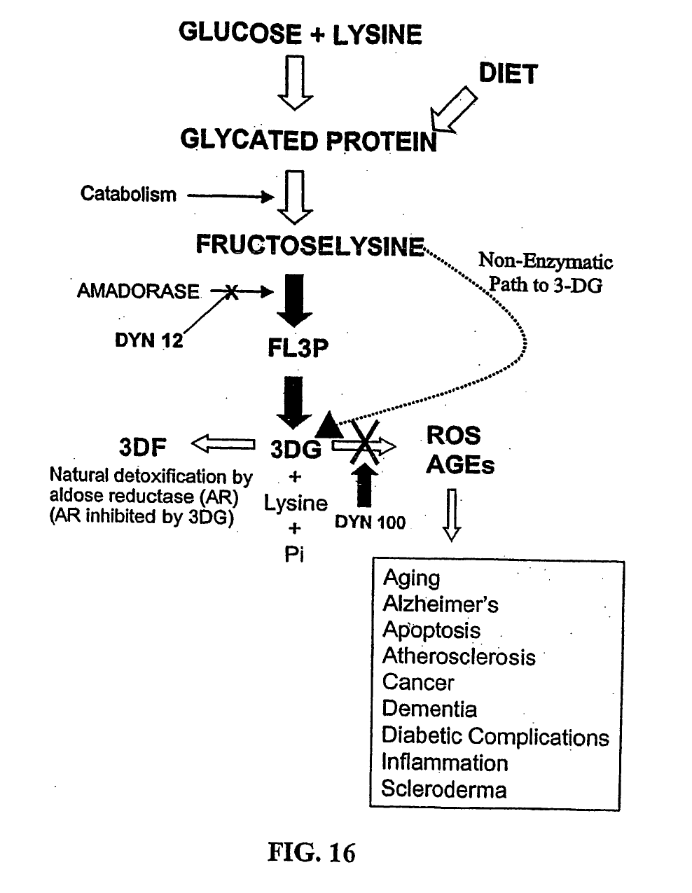

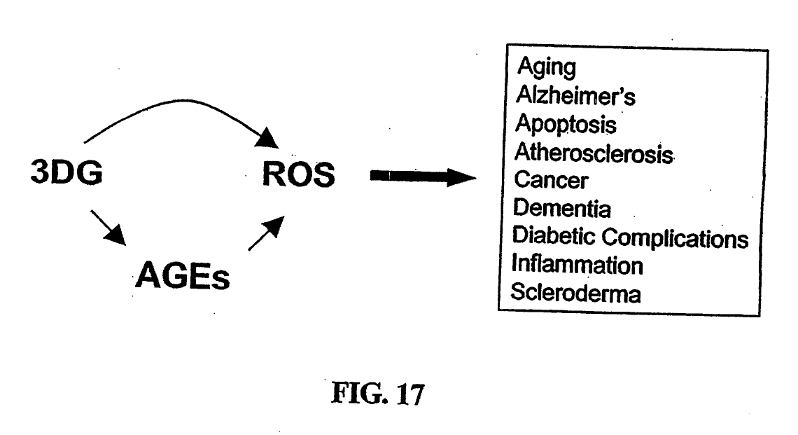

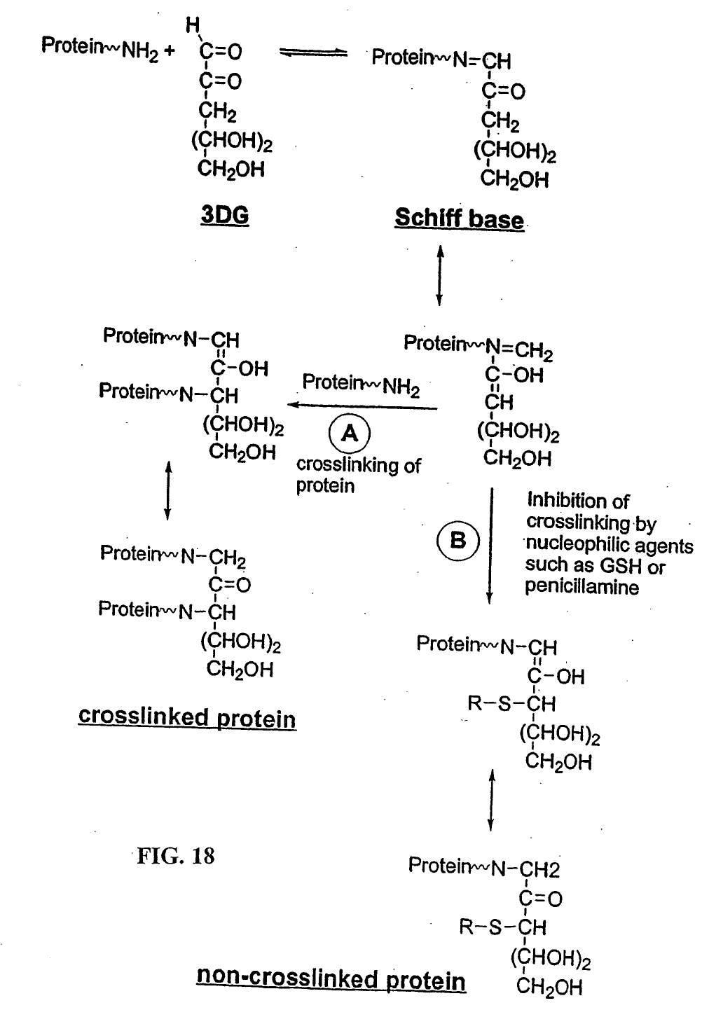

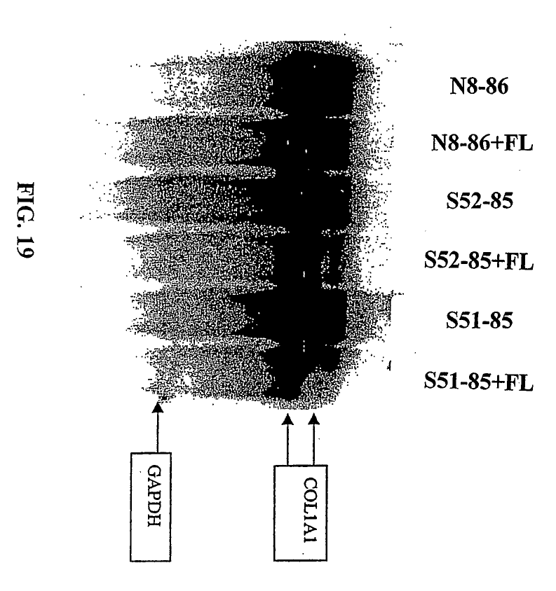

The term “AGE-proteins” (Advanced Glycation End product modified proteins), as used herein, refers to a product of the reaction between sugars and proteins [Brownlee, M. Glycation products and the pathogenesis of diabetic complications. 1992. Diabetes Care 15(12): p.1835-43; Niwa, T. et al. Elevated serum levels of 3-deoxyglucosone, a potent protein-cross-linking intermediate of the Maillard reaction, in uremic patients. 1995. Nephron 69(4): p.438-43]. For example, the reaction between protein lysine residues and glucose, this does not stop with the formation of fructoselysine (FL). FL can undergo multiple dehydration and rearrangement reactions to produce non-enzymatic 3DG, which reacts again with free amino groups, leading to crosslinking and browning of the protein involved. AGEs also include the products that form from the reaction of 3DG with other compounds, such as, but not limited to, as shown in FIG. 16.

“Amadorase,” as used herein, refers to a protein, fructosamine kinase, responsible for the production of 3DG. More specifically it refers to a protein which can enzymatically convert FL to FL3P, as defined above, when additionally supplied with a source of high energy phosphate. Additionally, this enzyme can convert fructose to fructose-3-phosphate when supplied with a source of high energy phosphate.

The term “Amadori product,” as used herein, refers to a ketoamine, such as, but not limited to, fructoselysine, comprising is a rearrangement product following glucose interaction with the ε-NH2 groups of lysine-containing proteins.

As used herein, “amino acids” are represented by the full name thereof, by the three-letter code corresponding thereto, or by the one-letter code corresponding thereto, as indicated in the following table:

| | Three-Letter | One-Letter |
|---|---|---|
| Full Name | Code | Code |
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biological sample,” as used herein, refers to samples obtained from a living organism, including skin, hair, tissue, blood, plasma, cells, sweat and urine.

The term “clearance,” as used herein refers to the physiological process of removing a compound or molecule, such as by diffusion, exfoliation, removal via the bloodstream, and excretion in urine, or via other sweat or other fluid.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug or a candidate for use as a drug, as well as combinations and mixtures of the above or modified versions or derivatives of the compound.

As used herein, the terms “conservative variation” or “conservative substitution” refer to the replacement of an amino acid residue by another, biologically similar residue. Conservative variations or substitutions are not likely to significantly change the shape of the peptide chain. Examples of conservative variations, or substitutions, include the replacement of one hydrophobic residue such as isoleucine, valine, leucine or alanine for another, or the substitution of one charged amino acid for another, such as the substitution of arginine for lysine, glutamic for aspartic acid, or glutamine for asparagine, and the like.

The term “Desmosines” as used herein refers to tetrafunctional crosslinks that are unique to elastin. Desmosine and Isodesmosine are formed from four Lys residues but only link two tropoelastin chains. Three allysines and one Lys residue contribute to each desmosine and isodesmosine. It is thought that the presence of an aromatic residue (Tyr or Phe) on the C-terminal side of Lys prevents oxidation by lysyl oxidase. This favors lysinonorleucine formation and thus directing desmosine and isodesmosine formation.

“Detoxification” of 3DG refers to the breakdown or conversion of 3DG to a form which does not allow it to perform its normal function. Detoxification can be brought about or stimulated by any composition or method, including “pharmacologic detoxification”, or metabolic pathway which can cause detoxification of 3DG. “Pharmacologic detoxification of “3DG” or other alpha-dicarbonyl sugars refers to a process in which a compound binds with or modifies 3DG, which in turn causes it to be become inactive or to be removed by metabolic processes such as, but not limited to, excretion.

The term “diabetes” as used herein refers to a metabolic disorder of multiple etiology characterized by chronic hyperglycemia with disturbances of carbohydrate, fat and protein metabolism resulting from defects in insulin secretion, insulin action, or both. “Diabetic complications” refer to retinopathy, nephropathy, neuropathy dementia and atherosclerosis.

A “disease” is a state of health of an animal wherein the animal cannot maintain homeostasis, and wherein if the disease is not ameliorated then the animal's health continues to deteriorate. As used herein, normal aging is included as a disease.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

The term “Elastin” as used herein refers to an insoluble protein found in the extracellular matrix of connective tissue, (including cartilage, bone, fat, and the tissue that supports the nerves and blood vessels throughout the body), providing elasticity and resilience to tissues that require the ability to deform repetitively and reversibly.

The term “elastin containing organs” as used herein refers to the extracellular matrix of connective tissue including by way of example the lungs, heart, intestines, blood vessels, skin and any other organ in the body that contains the protein elastin.

An “effective amount” or “therapeutically effective amount” of a compound is that amount of compound that is sufficient to provide a beneficial effect to the subject to which the compound is administered, or gives the appearance of providing a therapeutic effect as in a cosmetic.

As used herein, the term “effector domain” refers to a domain capable of directly interacting with an effector molecule, chemical, or structure in the cytoplasm which is capable of regulating a biochemical pathway.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “fibrosis” herein refers to scarring that can be the basis for one of the cardinal signs of inflammation, namely, loss of function. The loss can either be due to replacement of parenchymatous tissue (e.g., contractile heart muscle fibers) or to mechanical problems that scar tissue can produce. For example, as scar tissue matures, it contracts. It can, therefore, constrict organs that it surrounds (so-called napkin ring scarring or fibrosis of the intestine) or impede movement (e.g., when it crosses a joint).

The term “floating,” as used herein, refers to bonds of a substituent to a ring structure, such that the substituent can be attached to the ring structure at any available carbon juncture. A “fixed” bond means that a substituent is attached at a specific site.

The term “formation of 3DG” refers to 3DG, which is not necessarily formed via a synthetic pathway, but can be formed via a pathway such as spontaneous or induced breakdown of a precursor.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment,” as applied to a nucleic acid, can ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350even more preferably, at least about 350 nucleotides to about 500 nucleotides, yet even more preferably, at least about 500 to about 600even more preferably, at least about 600 nucleotides to about 620 nucleotides, yet even more preferably, at least about 620 to about 650and most preferably, the nucleic acid fragment will be greater than about 650 nucleotides in length.

The term “fructoselysine” (FL) is used herein to signify any glycated-lysine, whether incorporated in a protein/peptide or released from a protein/peptide by proteolytic digestion. This term is specifically not limited to the chemical structure commonly referred to as fructoselysine, which is reported to form from the reaction of protein lysine residues and glucose. As noted above, lysine amino groups can react with a wide variety of sugars. Indeed, one report indicates that glucose is the least reactive sugar out of a group of sixteen (16) different sugars tested [Bunn, H. F. and Higgins, P. J. Reaction of monosaccharides with proteins: possible evolutionary significance. 1981. Science 213(4504): p.222-4]. Thus, tagatose-lysine formed from galactose and lysine, analogously to glucose is included wherever the term fructoselysine is mentioned in this description, as is the condensation product of all other sugars, whether naturally-occurring or not. It will be understood from the description herein that the reaction between protein-lysine residues and sugars involves multiple reaction steps. The final steps in this reaction sequence involve the crosslinking of proteins and the production of multimeric species, known as AGE-proteins, some of which are fluorescent. Once an AGE protein forms, then proteolytic digestion of such AGE-proteins does not yield lysine covalently linked to a sugar molecule. Thus, these species are not included within the meaning of “fructoselysine”, as that term is used herein.

The term “Fructoselysine-3-phosphate,” as used herein, refers to a compound formed by the enzymatic transfer of a high energy phosphate group from ATP to FL. The term fructoselysine-3-phosphate (FL3P), as used herein, is meant to include all phosphorylated fructoselysine moieties that can be enzymatically formed whether free or protein-bound.

“Fructoselysine-3-phosphate kinase” (FL3K), as used herein, refers to one or more proteins, such as Amadorase, which can enzymatically convert FL to FL3P, as described herein, when supplied with a source of high energy phosphate. The term is used interchangeably with “fructoselysine kinase (FLK)”, fructosamine-3-kinase (F3K), and with “Amadorase”.

The term “Amadori Pathway,” or “Amadorase pathway” as used herein, refers to a lysine recovery pathway which exists in human skin, kidney, lung and other collagen containing organs, and possibly other tissues, which regenerates unmodified lysine as a free amino acid or as incorporated in a polypeptide chain or protein and includes substrates and products therefore, including methods or means which start or stimulate the pathway or events leading to the synthesis, production, or formation of lysine and 3DG. It is understood that the pathway includes the phosphorylation of fructose (fructose 3-kinase activity) without an amino acid attached to form fructose-3-phosphate, which in turn decomposes to yield 3DG.

The term “Glycated Diet,” as used herein refers to any given diet in which a percentage of normal protein is replaced with glycated protein. The expressions “glycated diet” and “glycated protein diet” are used interchangeably herein.

“Glycated lysine residues,” as used herein, refers to the modified lysine residue of a stable adduct produced by the reaction of a reducing sugar and a lysine-containing protein.

The majority of protein lysine residues are located on the surface of proteins as expected for a positively charged amino acid. Thus, lysine residues on proteins, which come in contact with serum, or other biological fluids, can freely react with sugar molecules in solution. This reaction occurs in multiple stages. The initial stage involves the formation of a Schiff base between the lysine free amino group and the sugar keto-group. This initial product then undergoes the Amadori rearrangement, to produce a stable ketoamine compound.

This series of reactions can occur with various sugars. When the sugar involved is glucose, the initial Schiff base product will involve imine formation between the aldehyde moiety on C-1 of the glucose and the lysine -amino group. The Amadori rearrangement will result in formation of lysine coupled to the C-I carbon of fructose, 1-deoxy-1- (aminolysine)-fructose, herein referred to as fructoselysine or FL. Similar reactions will occur with other aldose sugars, for example galactose and ribose [Dills, W. L., Jr. Protein fructosylation: fructose and the Maillard reaction. 1993. Am J. Clin Nutr 58(5 Suppl): p.779S-787S]. For the purpose of the present invention, the early products of the reaction of any reducing sugar and the y-amino residue of protein lysine are included within the meaning of glycated-lysine residue, regardless of the exact structure of the modifying sugar molecule.

“Guanidino” as used herein is —N(R″)—C(═NH)—NH2 where R″ represents H, or a linear or branched chain alkyl group (C1-C4).

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10are matched or homologous, the two sequences share 90% homology. By way of example, the DNA sequences 3′ATTGCC5′ and 3′TATGGC share 50% homology.

As used herein, “homologous” or homology” are used synonymously with “identity”. The determination of percent identity or homology-between two nucleotide or amino acid sequences can be accomplished using a mathematical algorithm. For example, a mathematical algorithm useful for comparing two sequences is the algorithm of Karlin and Altschul (Altschul et al., 1990Proc. Natl. Acad. Sci. U.S.A. 87:5509-13) modified as in Karlin and Altschul (Karlin et al., 1993Proc. Natl. Acad. Sci. U.S.A. 90:5873-7). This algorithm is incorporated into the NBLAST and XBLAST programs of Altschul, et al. (Altschul et al., 1990J. Mol. Biol. 215:403-10) and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0BLOSUM62 scoring matrix to obtain amino acid sequences homologous to a protein molecule described herein. To obtain gapped alignments for comparison purposes, Gapped BLAST can be utilized as described in Altschul et al. (1997(Altschul et al., 1997Nucleic Acids Res. 25:3389-402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

“Inhibiting 3DG” as described herein, refers to any method or technique, which inhibits 3DG synthesis, production, formation, accumulation, or function, as well as methods of inhibiting the induction or stimulation of synthesis, formation, accumulation, or function of 3DG. It also refers to any metabolic pathway, which can regulate 3DG function or induction. The term also refers to any composition or method for inhibiting 3DG function by detoxifying 3DG or causing the clearance of 3DG. Inhibition can be direct or indirect. Induction refers to induction of synthesis of 3DG or to induction of function. Similarly, the phrase “inhibiting alpha-dicarbonyl sugars”, refers to inhibiting members of the alpha-dicarbonyl sugar family, including 3DG, glyoxal, methyl glyoxal, and glucosone.

The term “inhibiting accumulation of 3DG,” as used herein, refers to the use of any composition or method which decreases synthesis, increases degradation, or increases clearance, of 3DG such that the result is lower levels of 3DG or functional 3DG in the tissue being examined or treated, compared with the levels in tissue not treated with the composition or method. Similarly, the phrase “inhibiting accumulation of alpha-dicarbonyl sugars”, refers to inhibiting accumulation of members of the alpha-dicarbonyl sugar family, including 3DG, glyoxal, methyl glyoxal, and glucosone, and intermediates thereof.

As used herein, an “instructional material” includes a publication, a recording, a diagram, or any other medium of expression, which can be used to communicate the usefulness of the peptide of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container that contains the identified compound invention or be shipped together with a container, which contains the identified compound. Alternatively, the instructional material can be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids that have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA, which is part of a hybrid gene encoding additional polypeptide sequence.

The term “lupus” as used herein refers to a chronic, often life-long, autoimmune disease that ranges from mild to severe and afflicts mostly women. Systemic lupus erythematosus (SLE) may affect widespread sites, but it most often manifests in the skin, joints, blood, and kidneys.

“Modified” compound, as used herein, refers to a modification or derivation of a compound, which may be a chemical modification, such as in chemically altering a compound in order to increase or change its functional ability or activity.

The term “mutagenicity” refers to the ability of a compound to induce or increase the frequency of mutation. The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequences (i.e., A, T, G, C), this also includes an RNA sequence (i.e., A, U, G, C) in which “U” replaces “T.”

The term “peptide” typically refers to short polypeptides.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Polypeptide” refers to a polymer composed of at least two amino acid residues, related naturally occurring structural variants, and synthetic non-naturally occurring analogs thereof linked via peptide bonds, related naturally occurring structural variants, and synthetic non-naturally occurring analogs thereof.

A “polynucleotide” means a single strand or parallel and anti-parallel strands of a nucleic acid. Thus, a polynucleotide may be either a single-stranded or a double-stranded nucleic acid.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence that is required for expression of a gene product operably linked to the promoter/regulator sequence. In some instances, this sequence may be the core promoter sequence and in other instances, this sequence may also include an enhancer sequence and other regulatory elements, which are required for expression of the gene product. The promoter/regulatory sequence may, for example, be one that expresses the gene product in a tissue specific manner.

A “constitutive” promoter is a promoter, which drives expression of a gene to which it is operably linked, in a constant manner in a cell. By way of example, promoters that drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

The term “protein” typically refers to large polypeptides.

The term “Reactive Oxygen Species” includes various harmful forms of oxygen are generated in the body; singlet oxygen, superoxide radicals, hydrogen peroxide, and hydroxyl radicals, all of which can cause tissue damage. A catchall term for these and similar oxygen related species is “reactive oxygen species” (ROS). The term also includes ROS formed by the internalization of AGEs into cells and the ROS that form therefrom.

“Removing 3-deoxyglucosone”, as used herein, refers to any composition or method, the use of which results in lower levels of 3-deoxyglucosone (3DG) or lower levels of functional 3DG when compared to the level of 3DG or the level of functional 3DG in the absence of the composition. Lower levels of 3DG can result from its decreased synthesis or formation, increased degradation, increased clearance, or any combination of thereof. Lower levels of functional 3DG can result from modifying the 3DG molecule such that it can function less efficiently in the process of glycation or can result from binding of 3DG with another molecule which blocks inhibits the ability of 3DG to function. Lower levels of 3DG can also result from increased clearance and excretion in urine of 3DG. The term is also used interchangeably with “inhibiting accumulation of 3DG”. Similarly, the phrase “removing alpha-dicarbonyl sugars”, refers to removal of members of the alpha-dicarbonyl sugar family, including 3DG, glyoxal, methyl glyoxal, and glucosone.

Also, the terms glycated-lysine residue, glycated protein and glycosylated protein or lysine residue are used interchangeably herein, is consistently with current usage in the art where such terms are art-recognized used interchangeably.

The term “protein crosslinking” refers to a covalent binding of a protein or peptide to itself or to one or more other proteins or peptides. These protein crosslinked bonds are not normal to the natural physiological state or function of the protein or proteins and can result in the inactivation and/or precipitation of the protein(s). These crosslinks can be broken by the use of compositions or compounds called “crosslink breakers.” An example of such a crosslink breaker is Alteon's ALT-711 (Vasan et al., 2003Arch. Biochem. Biophys. 419:89-96).

The term “scleroderma” as used herein refers to a progressive disease that affects the skin and connective tissue (including cartilage, bone, fat, and the tissue that supports the nerves and blood vessels throughout the body). Scleroderma is an autoimmune disorder, which means that the body's immune system turns against itself. In scleroderma, there is an overproduction of abnormal collagen (a type of protein fiber present in connective tissue). This collagen accumulates throughout the body, causing hardening (sclerosis), scarring (fibrosis), and other damage. The damage may affect the appearance of the skin, or it may involve only the internal organs. The symptoms and severity of scleroderma vary from person to person.

The term “skin,” as used herein, refers to the commonly used definition of skin, e.g., the epidermis and dermis, and the cells, glands, mucosal and connective tissue which comprise the skin.

The term “standard,” as used herein, refers to something used for comparison. For example, it can be a known standard agent or compound which is administered and used for comparing results when administering a test compound or it can be a standard parameter or function which is measured to obtain a control value when measuring an effect-of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not limited to, a purified marker of interest which has been labeled, such as with a radioactive isotope, allowing it to be distinguished from an endogenous substance in a sample.

A “susceptible test animal,” as used herein, refers to a strain of laboratory animal which, due to for instance the presence of certain genetic mutations, have a higher propensity toward a disease disorder or condition of choice, such as diabetes, cancer, and the like.

“Synthesis of 3DG”, as used herein refers to the formation or production of 3DG. 3DG can be formed based on an enzyme dependent pathway or a non-enzyme dependent pathway. Similarly, the phrase “synthesis of alpha-dicarbonyl sugars”, refers to synthesis or spontaneous formation of members of the alpha-dicarbonyl sugar family, including 3DG, glyoxal, methyl glyoxal, and glucosone, and adducts as disclosed herein.

“Synthetic peptides or polypeptides” mean a non-naturally occurring peptide or polypeptide. Synthetic peptides or polypeptides can be synthesized, for example, using an automated polypeptide synthesizer. Those of skill in the art know of various peptide synthesis methods.

A “therapeutic” treatment is a treatment administered to a subject who exhibits signs of pathology, for the purpose of diminishing or eliminating those signs.

By “transdermal” delivery is intended either transdermal (or “percutaneous”) and transmucosal administration, i.e., delivery by passage of a drug through the skin or mucosal tissue and into the bloodstream. Transdermal also refers to the skin as a portal for the administration of drugs or compounds by topical application of the drug or compound thereto.

The term “topical application”, as used herein, refers to administration to a surface, such as the skin. This term is used interchangeably with “cutaneous application”.

The term to “treat,” as used herein, means reducing the frequency with which symptoms are experienced by a patient or subject or administering an agent or compound to reduce the frequency with which symptoms are experienced.

As used herein, “treating a disease or disorder,” means reducing the frequency with which a symptom of the disease or disorder is experienced by a patient. Disease and disorder are used interchangeably herein.

The term tropoelastin” as used herein refers to the soluble precursor to elastin. Tropoelastin reinforces the high-pressure closed circulatory systems of higher vertebrates.

As used herein, the term “wild-type” refers to the genotype and phenotype that is a characteristic of most of the members of a species occurring naturally and contrasting with the genotype and phenotype of a mutant.

The term “modulate,” as used herein, refers to the alteration of a process or activity from one state or condition to another. For example, modulation of 3DG activity includes the increased activity of 3DG by way of an increased concentration of 3DG. At the same time, modulation of 3DG activity also includes the decreased activity of 3DG through inhibition of 3DG production. Further, the activity, level, concentration, or effect of a composition, compound, polypeptide, or the like, may be “enhanced,” as the term is used herein, if the activity, level, concentration, or effect of a composition, compound, polypeptide, or the like, is greater relative to a comparative reference value of the activity, level, concentration, or effect of a composition, compound, polypeptide, or the like.

An “analog” of a compound, as the term is used herein, refers to a second compound that has some or all of the properties of a first compound. An analog may be a functional analog, a structural analog, or both. The properties of an analog may be lesser than, equal to, or greater than the corresponding properties of the compound of which it is an analog.

“Detoxification” of 3DG, as the term is used herein, refers to the alteration, inactivation, or removal of 3DG from a mammal. For example, 3DG may be detoxified by chemical conversion of 3DG to a new chemical entity, either by addition or removal of one or more atoms or molecules to or from 3DG.

“Stabilizing” refers to the maintenance of a state or condition at or near its current status.

The invention relates generally to the novel discovery that modulating the Amadorase pathway results in changes in levels of mRNA for collagen and in the formation of desmosines, the essential elements of elastin. The invention is further based on the knowledge that the levels of mRNA for collagen type I in certain diseases, are elevated, and that these levels can be reduced by methods and compounds that affect the Amadorase pathway. By way of example only, these diseases include scleroderma, endomyocardial fibrosis, pulmonary fibrosis, ARDS, and cGvH. The invention therefore encompasses methods and compounds that increase the flux through the Amadorase pathway so that mRNA for collagen type I is reduced, thereby reducing the level of collagen type I production and the effects of any related diseases. This is especially important in the treatment of scleroderma and keloids, two diseases characterized by excessive amounts of collagen production. Following treatment with compounds to increase the flux through the Amadorase pathway, it is important to eliminate any 3DG that forms. This can be accomplished by an efficient detoxification means and/or enhancing the detoxification of 3DG or by inactivating 3DG. Preferably the methods used to increase the flux through the Amadorase pathway do not result in the formation of 3DG. Compounds that increase the flux through the Amadorase pathway include glycated proteins and Amadori compounds such as fructoselysine, tagatose lysine, and morpholinofructose and the sugar frutose.

As discussed in detail elsewhere herein, increased collagen levels characterize many diseases. Nowhere is it taught that the increased levels of collagen can be lowered by increasing the flux through the fructosamine 3 kinase pathway. It is hypothesized that in response to the body making more collagen, the fructosamine 3 kinase pathway is activated so that the levels of collagen type I mRNA are significantly decreased, resulting in less collagen. Unfortunately, the continued increase in the flux through the pathway results in the accumulation of the toxic compound 3 deoxyglucosone which causes oxidative stress, the formation of collagen crosslinks and the formation of advanced glycation end products. Compounds that cause the formation of fructose lysine 3 phosphate and fructose lysine 3 phosphate-like compounds will cause a decrease in the levels of collagen mRNA, as well as substrates for the enzyrne. However, in the event that the substrate or product of the enzyme result in the production of 3DG, it is necessary to administer another compound that inactivates 3DG, or another compound that is bifunctional. In the alternative, one could inhibit the enzyme to decrease the formation of 3DG, and not get the benefit of decreasing collagen type I mRNA.

Prior to the present invention, disclosed herein for the first time, it was not known that modulating the Amadori Pathway affects the formation of mRNA for collagen and that levels of mRNA for collagen can be modulated by changing the flux through the Amadori Pathway, so that increasing the flux results in less type I collagen production and decreasing the flux results in more type I collagen production. Also, nowhere is it disclosed that levels of mRNA for collagen can be controlled by methods and compounds to inhibit the formation of FL, the enzyme fructosamine-3-kinase, and 3DG and nowhere is it disclosed that the Amadori Pathway can be regulated to inhibit the synthesis of collagen to prevent and/or treat scleroderma and related inflammatory diseases.

When armed with the disclosure set forth herein for the first time, the skilled artisan will therefore understand that a patient having a disease resulting in an excess of collagen may benefit from administration of an activator of the Amadorase pathway. In one embodiment of the invention, an activator of the Amadorase pathway can subsequently decrease the mRNA for collagen, thereby decreasing collagen production in the patient. Alternatively, a patient having a disease resulting in a collagen deficiency may benefit from administration of an inhibitor of the Amadorase pathway. In another embodiment of the invention, an inhibitor of the Amadorase pathway can subsequently increase the mRNA for collagen, thereby increasing the collagen production in a patient.

Furthermore, it is shown herein for the first time that modulating the Amadori Pathway affects the formation of desmosines in diabetics, and that desmosine levels can be decreased by inhibition of the Amadori Pathway.

Therefore, the invention further encompasses compositions and methods to inhibit 3DG formation or to remove 3DG from the extracellular matrix and organs containing collagen, as well as compositions and methods to increase the rate of detoxification and removal of 3DG from said extracellular matrix and collagen containing organs. Furthermore, the invention encompasses compositions and methods to break apart 3DG-protein/peptide adducts present in crosslinked collagen, elastin and other proteins.

These compounds would be used in conjunction with compounds to increase the flux through the Amadorase pathway in order to decrease the chance of unwanted side effects from the 3DG that may form.

The invention therefore encompasses methods and compositions to prevent and treat the complications of certain inflammatory diseases associated with elevated levels of mRNA for collagen type I, which diseases include scleroderma and keloids. The invention also encompasses methods and compositions to prevent and treat certain collagen related disease. Collagen related diseases include those listed above.

Until the present invention, regulation of mRNA of type I was not associated with the Amadorase pathway. The data disclosed herein demonstrate, for the first time that collagen mRNA can be regulated by varying the flux through the Amadorase pathway. Increasing the flux through the Amadorase pathway results in the production of less mRNA for type I collagen in skin. Inhibiting the flux through the Amadorase pathway results in less mRNA for type I collagen and less collagen in skin.

Conditions and diseases also exist wherein the levels of type I collagen decrease, such as in aging skin and arteries. Under such circumstances, inhibiting the flux through the Amadorase pathway to increase the levels of mRNA for collagen type I would be beneficial to prevent the decrease in type I collagen and prevent, by way of example, the thinning of skin and wrinkles associated with age and the thinning of blood vessels and arteries associated with aging. The invention therefore encompasses compositions and methods to inhibit the flux through the enzyme fructosamine 3 kinase, inhibiting the enzyme fructosamine 3 kinase and inhibiting the formation of 3DG, as well as inactivating 3DG. Compounds that inhibit the enzyme and compounds that inactivate 3DG are set forth elsewhere herein, and are also referenced, in part, in International Patent Application number PCT/US03/12003 and U.S. Pat. No. 6,006,958, incorporated herein by reference.

Elastin

The invention is further based on the discovery that the levels of desmosines, in diabetes, are elevated, and that these levels can be reduced by methods and compounds that affect the Amadorase Pathway. The invention therefore encompasses compositions and methods to inhibit the flux through the enzyme fructoseamine 3 kinase, inhibiting the enzyme fructoseamine 3 kinase and inhibiting the formation of 3DG, as well as inactivating 3DG. Compounds that inhibit the enzyme and compounds that inactivate 3DG are set forth hereinafter, and referenced in International Publication Number WO 03/089601, having an International Patent Application number of PCT/US03/12003 and U.S. Pat. No. 6,006,958, incorporated herein.

The invention further encompasses compositions and methods to inhibit 3DG formation or to remove 3DG from organs containing elastin, as well as compositions and methods to increase the rate of detoxification and removal of 3DG from organs containing elastin.

The invention is further based on the concept that development of inelastic aged skin and inelastic elastin containing organs can be prevented and reversed by compositons and methods that inhibit the formation of desmosines by inhibiting the flux through the enzyme fructoseamine 3 kinase, inhibiting the enzyme fructoseamine 3 kinase and inhibiting the formation of 3DG, as well as inactivating.

Inelastic containing organs include the extracellular matrix that forms the internal structure of the body and its organs, and more specifically skin, lungs, ligament, blood vessels, and elastic cartilage.

The invention also encompasses methods and compositions to prevent and treat certain elatin related disease. Elastin related diseases include artherosclerosis, Buscke-Oljlendorff syndrome, Cutis laxa, emphesema, Marfan syndrome, Menkes syndrome, Pseudoxanthoma elasticum, Supravalvular aortic stenosis and Williams syndrome.

Methods of Inhibiting the Amadorase Pathway

One skilled in the art could conceive of many ways to modulate the Amadorase pathway. These include antibodies. The antibody can be an antibody that is known in the art or it can be an antibody prepared using known techniques and the published sequence of the fructosamine kinase/Amadorase (Accession No. NP_{—}071441). In one aspect, the antibody is selected from the group consisting of a polyclonal antibody, a monoclonal antibody, a humanized antibody, a chimeric antibody, and a synthetic antibody.

In another embodiment of the invention, fructosamine kinase function can be inhibited by using antisense or siRNA gene silencing techniques. In one embodiment, antisense nucleic acids complementary to fructosamine kinase mRNA can be used to block the expression or translation of the corresponding mRNA (see SEQ ID NO:1) (see Examples 20 and 22).

In another embodiment, an siRNA to the fructosamine kinase mRNA can be used to knockdown the expression of the protein triggered by the introduction of double-stranded RNA (dsRNA) which leads to gene silencing in a sequence-specific manner.

The invention should not be construed to include only fructosamine kinase inhibition using antisense or siRNA techniques, but should also be construed to include inhibition or upregulation of other genes and their proteins that are involved in the Amadori Pathway.

Using Compounds to decrease the levels of mRNA for collagen type I. In one embodiment the invention includes a method of increasing the levels of mRNA for collagen type I, said method comprising administering to a mammal an effective amount of an inhibitor of FL3K synthesis, or a derivative or modification thereof, thereby lowering levels of mRNA for collagen type I.

In one embodiment, the invention includes a method of inhibiting desmosine levels, said method comprising administering to a mammal an effective amount of an inhibitor of desmosine synthesis, or a derivative or modification thereof, therby inhibiting desmosine synthesis.

As discussed in detail elsewhere herein, a desmosine inhibitor can comprise from about 0.0001% to about 15% by weight of the pharmaceutical composition. In one aspect, the inhibitor is administered as a controlled-release formulation. In another aspect the pharmaceutical composition comprises a lotion, a cream, a gel, a liniment, an ointment, a paste, toothpaste, a mouthwash, an oral rinse, a coating, a solution, a powder, and a suspension. In yet another aspect, the composition further comprises a moisturizer, a humectant, a demulcent, oil, water, an emulsifier, a thickener, a thinner, a surface active agent, a fragrance, a preservative, an antioxidant, a hydrotropic agent, a chelating agent, a vitamin, a mineral, a permeation enhancer, a cosmetic adjuvant, a bleaching agent, a depigmentation agent, a foaming agent, a conditioner, a viscosifier, a buffering agent, and a sunscreen.

Also as discussed in greater detail elsewhere herein, the invention should be construed to include various methods of administration, including topical, oral, intramuscular, and intravenous.

In one aspect of the invention, the inhibitor of FL3K is a compound such as those of the formula (Formula XIX): 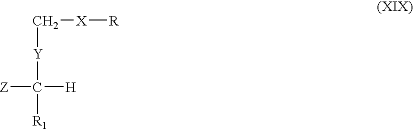
wherein X is a divalent moiety selected from the group consisting of —NR′—, —S(O)—, —S(O)2—, or —O—, R′ being selected from the group consisting of H, linear or branched chain alkyl group (C1-C4) an unsubstituted or substituted aryl group (C6-C10) or aralkyl group (C7-C10) or CH2(CHOR2)nCH2OR2 with n being 1-5 or CH(CH2OR2)(CHOR2)nCH2OR2 with n being 1-4 where R2 is H, alkyl (C1-C4) or an unsubstituted or substituted aryl group (C6-C10) or araalkyl group (C7-C10); R is a substituent selected from the group consisting of H, an amino acid residue, said amino acid including said NR′ moiety, a polyaminoacid residue, said polyamino acid including said NR′ moiety, a peptide chain, a linear or branched chain aliphatic group (C1-C8), which is unsubstituted or substituted with at least one nitrogen—or oxygen-containing substituent, a linear or branched chain aliphatic group (C1-C8), which is unsubstituted or substituted with at least one nitrogen- or oxygen-containing substituent and interrupted by at least one —O—, —NH—, or —NR3—moiety, R3 being linear or branched chain alkyl group (C1-C6) and an unsubstituted or substituted aryl group (C6-C10) or aralkyl group (C7-C10), with the proviso that when X represents—NR1-, R and R1, together with the nitrogen atom to which they are attached, may also represent a substituted or unsubstituted heterocyclic ring having from 5 to 7 ring atoms, with at least one of nitrogen and oxygen being the only heteroatoms in said ring, said aryl group (C6-C10) or aralkyl group (C7-C10) and said heterocyclic ring substituents being selected from the group consisting of H, alkyl (C1-C6), halogen, CF3, CN, NO2 and —0-alkyl (C1-C6); R1 is a polyol moiety having 1 to 4 linear carbon atoms, Y is either a carbonyl moiety or a hydroxymethylene moiety; Z is selected from a group consisting of—H, —O-alkyl (C1-C6), halogen —CF3, —CN, —COOH, and —SO3H2 and the stereoisomers and pharmaceutically acceptable salts of the said compound.

Other appropriate reactants include without limitation unsubstituted or substituted aryl (C6-C10) compounds, wherein the substituent may be alkyl (C1-C3), alkoxy, carboxy, nitro or halogen groups, unsubstituted or substituted alkanes, wherein the substituent may be at least one alkoxy group; or unsubstituted or substituted nitrogen-containing heterocyclic compounds, wherein the substituents may be alkyl (C1-C3), aryl (C6-C10), alkoxy, carboxy, nitro or halogen groups. Illustrative examples of the last-mentioned group of reactants include m-methyl-, p-methyl-, m-methoxy-, o-methoxy- and m-nitro-aminobenzenes, o- and p-aminobenzoic acids; n-propylamine, n-butylamine, 3-methoxypropylamine; morpholine and piperdine.

In one aspect of the invention, representative inhibitor compounds having the above formula include galactitol lysine, 3-deoxy sorbitol lysine, 3-deoxy-3-fluoro-xylitol lysine, and 3-deoxy-3-cyano sorbitol lysine and 3-O-methyl sorbitollysine. Examples of known compounds that may be used as inhibitors in practicing this invention include, without limitation, meglumine, sorbitol lysine, galactitol lysine, and mannitol lysine. A preferred inhibitor is 3-O-methyl sorbitollysine.

The compounds of the invention may be administered to, for example, a cell, a tissue, or a subject by any of several methods described herein and by others that are known to those of skill in the art.

The invention should not be construed to include only the modifications, derivatives, or substitutions of Formula XIX and the representative compounds described herein. The invention should also be construed to include other modifications not described herein, as well as compounds not described herein, which are representative of Formula XIX.

In another aspect of the invention, the inhibitor of fructoseamine 3 kinase activity is a compound or complex containing copper or other metal including but not limited to zinc, aluminum, indium, manganese, titanium, platinum, gold or tin. Copper containing compounds or complexes suitable as inhibitors of the enzyme fructosamine 3 kinase are referenced in [Sorenson, J R Copper complexes offer a physiological approach to treatment of chronic diseases. 1989 Prog Med Chem 26:437; Pickart LR U.S. Pat. No. 5,554,375; Konishi U.S. Pat No. 4,461,724; Fairlie DP and Whitehouse MW 1991 Drug Des Discov 8:83-102], and are incorporated herein by reference. By way of a non-limiting example copper compounds and complexes useful in the present invention include copper salts, and complexes with amino acids, peptides (Cu(II):Gly-Ser-His-Lys) and organic molecules (Cu(II): 3,5-diisopropylsalicylate).

In another aspect of the present invention, flux through the Amadorase pathway may be increased by chelating copper or a copper-containing compound, so that the copper is not available as an inhibitor of the Amadorase pathway. In one embodiment, the present invention includes compositions and methods of chelating copper, such that the copper is not available as an inhibitor of the Amadorase pathway, thereby increasing the flux through the Aniadorase pathway.

In one aspect, the invention features a method comprising administration of a composition to a patient in need of activation of the Amadorase pathway, wherein the composition comprises a copper chelator. Copper chelators useful in the present invention include, but are not limited to, triethylenetetramine dihydrochloride (triene), penicillamine, sar, diamsar, ethylenediamine tetraacetic acid, o-phenanthroline, and histidine. Other copper chelators useful in the present invention include those described in U.S. Pat. No. 6,610,693, hereby incorporated by reference.

In one aspect of the invention, an inhibitor of the invention that lowers levels of messenger RNA for collagen type I may be synthesized in vitro using techniques known in the art (see, for example, Experimental Examples 27 and 28).

Compounds and Methods for Inhibiting 3DG and 3DG Production

The present invention features compounds and methods for inhibiting 3DG and 3DG production. Such compounds and methods can be used in conjunction with compounds that increase the flux through the Amadorase pathway

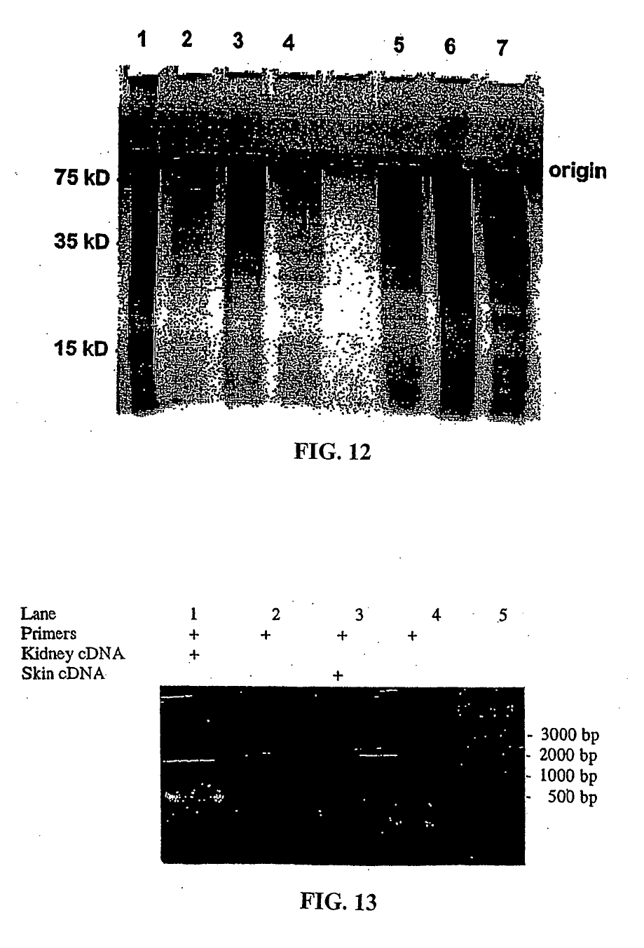

As described above, inhibition of 3DG function can be direct or indirect. Therefore, 3DG function may be inhibited or caused to decrease using many approaches as described elsewhere herein in greater detail. Inhibition of 3DG function may be assayed or monitored using techniques described herein as well as others known to those of skill in the art. Function can be measured directly or it can be estimated using techniques to measure parameters which are known to be correlative of 3DG function. For example, protein crosslinking and protein production can be measured directly using techniques such as electrophoretic analysis (see FIG. 12 and Experimental Examples 7 and 18) as well as other techniques (see Experimental Examples 21-24). The invention should be construed to include not only compounds useful for preventing 3DG induced crosslinking of molecules such procollagen and collagen but it should also be construed to include compounds which inhibit crosslinking of other molecules as well.

In one embodiment, the inhibitor comprises from about 0.0001% to about 15% by weight of the pharmaceutical composition. In one aspect, the inhibitor is administered as a controlled-release formulation. In another aspect the pharmaceutical composition comprises a lotion, a cream, a gel, a liniment, an ointment, a paste, toothpaste, a mouthwash, an oral rinse, a coating, a solution, a powder, and a suspension. In yet another aspect, the composition further comprises a moisturizer, a humectant, a demulcent, oil, water, an emulsifier, a thickener, a thinner, a surface active agent, a fragrance, a preservative, an antioxidant, a hydrotropic agent, a chelating agent, a vitamin, a mineral, a permeation enhancer, a cosmetic adjuvant, a bleaching agent, a depigmentation agent, a foaming agent, a conditioner, a viscosifier, a buffering agent, and a sunscreen.

The invention should be construed to include various methods of administration, including topical, oral, intramuscular, subcutaneous and intravenous.

By way of a non-limiting example, an inhibitor of 3DG function may be an isolated nucleic acid encoding a nucleic acid which is complementary to a fructosamine kinase mRNA and in an antisense orientation. Other inhibitors include an antisense oligonucleotide, an antibody, or other compounds or agents such as small molecules.

A method of the invention also includes use of the following compounds, as illustrated by their structural formulas, to inhibit or block 3DG function. Compounds which may be used in the practice of this invention include one or more (i. e., combinations) of the following:

Formula I comprises a structure wherein R1 and R2 are independently hydrogen, lower alkyl, lower alkoxy or an aryl group, or together with the nitrogen atom form a heterocyclic ring containing from 1 to 2 heteroatoms and 2 to 6 carbon atoms, the second of said heteroatoms being selected. from the group consisting of nitrogen, oxygen and sulfur, and includes their biocompatible and pharmaceutically acceptable acid addition salts.

The lower alkyl groups in the compounds of Formula (I) contain 1-6 carbon atoms and include methyl, ethyl, propyl, butyl, pentyl, hexyl, and the corresponding branched chain isomers thereof. The lower alkoxy groups have 1-6 carbon atoms and include methoxy, ethoxy, propoxy, butoxy, penthyloxy, and hexyloxy and branched chain isomers thereof. The aryl groups include both substituted and unsubstituted phenyl and pyridyl groups. Typical aryl group substituents are those such as lower alkyl groups, fluoro, chloro, bromo, and iodo atoms. 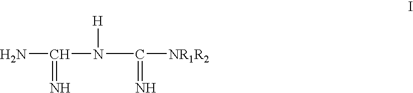
Of the compounds encompassed by Formula I, certain combinations of substituents are preferred. For instance, when R, is a hydrogen atom, then R2 is preferably hydrogen or an aryl group.

When R and R2 are both alkyl groups, then the compounds having identical R and R2 alkyl groups are preferable.

When R, and R2 together with the nitrogen atom form a heterocyclic ring containing from 1 to 2 heteroatoms, said heteroatoms being selected from the group consisting of nitrogen, oxygen and sulfur, the preferred heterocyclic rings will be morpholino, piperazinyl, piperidinyl and thiomorpholino, with the morpholino being most preferred.

Representative of the compounds of formula (I) are:
N,N-dimethylimidodicarbonimidic diamide; imidodicarbonimidic diamide;
N-phenylimidodicarbonimidic diamide;
N-(aminoiminomethyl)-4-morpholinecarboximidamide;
N-(aminoiminomethyl)-4-thiomorpholinecarboximidamide;
N-(aminoiminomethyl)-4-methyl-1-piperazinecarboximidamide;
N-(aminoiminomethyl)-1-piperidinecarboximidamide;
N-(aminoiminomethyl)-1-pyrrolidinecarboximidamide;
N-(aminoiminomethyl)-I-hexahydroazepinecarboximidamide;(aminoiminomethyl)-I-hexahydroazepinecarboximidamide
N-4-pyridylimidodicarbonimidic diamide;
N,N-di-n-hexylimidodicarbonimidic diamide;
N,N-di-n-pentylimidodicarbonimidic diamide;
N,N-d-n-butylimidodicarbonimidic diamide;
N,N-dipropylimidodicarbonimidic diamide;
N,N-diethylimidodicarbonimidic diamide; and the pharmaceutically acceptable acid addition salts thereof.

Formula II comprises a structure wherein Z is N or CH—; X, Y and Q are each independently a hydrogen, amino, heterocyclo, amino lower alkyl, lower alkyl or hydroxy group, and R3 is hydrogen or an amino group, their corresponding 3-oxides, and includes their biocompatible and pharmaceutically acceptable salts.

The compounds of Formula II, wherein the X, Y or Q substituent is on a nitrogen of the ring, exist as tautomers, i. e., 2-hydroxypyrimidine can exist also as 2 (1H)-pyrimidine. Both forms may be used in practicing this invention.

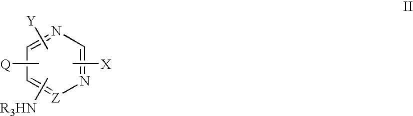

The lower alkyl groups of the compounds of formula II contain 1-6 carbon atoms and include methyl, ethyl, propyl, butyl, pentyl, hexyl, and the corresponding branched chain isomers thereof. The heterocycylic groups of the compounds of formula II contain from 3-6 carbon atoms and are exemplified by groups such as pyrrolidinyl, -methylpyrrolidinyl, piperidinol, 2-methylpiperidino morpholino, and hexamethyleneamino.

The “floating” X, Y, Q and NHR3 bonds in Formula II indicate that these variants can be attached to the ring structure at any available carbon juncture. The hydroxy variant of X, Y and Q can also be present on a nitrogen atom.

Of the compounds encompassed by Formula II, certain combinations of substituents are preferred. For instance, compounds having R3 as hydrogen, as a CH group, and at least one of X, Y or Q as another amino group, are preferred. The group of compounds where R3 is hydrogen, Z is a CH group and one of X or Y is an amino lower alkyl group are also preferred. Another preferred group of compounds is those where R is hydrogen and Z is N (nitrogen). Certain substitution patterns are preferred, i. e., the 6-position (IUPAC numbering, Z. dbd. CH) is preferably substituted, and most preferably by an amino or a nitro containing group. Also preferred are compounds where two or more of X, Y and Q are other than hydrogen.

Representative of the compounds of formula II are:
4,5-diaminopyrimidine; 4-amino-5-aminomethyl-2-methylpyrimidine; 6-(piperidino)-2,4-diaminopyrimidine 3-oxide; 4,6-diaminopyrimidine; 4,5,6-triaminopyrimidine; 4,5-diamino-6-hydroxy pyrimidine; 2,4,5-triamino-6-hydroxypyrimidine; 2,4,6-triaminopyrimidine; 4,5-diamino-2-methylpyrimidine; 4,5-diamino-2,6-dimethylpyrimidine; 4,5-diamino-2-hydroxy-pyrimidine; and 4,5-diamino-2-hydroxy-6-methylpyrimidine.

Formula III comprises a structure wherein R4 is hydrogen or acyl, R5 is hydrogen or lower alkyl, Xa is a substituent selected from the group consisting of lower alkyl, carboxy, carboxymethyl, or a phenyl or pyridyl group, optionally substituted by halogen, lower alkyl, hydroxy lower alkyl, hydroxy, or acetylamino with the proviso that when X is a phenyl or pyridyl group, optionally substituted, then R5 is hydrogen and includes their biocompatible and pharmaceutically acceptable acid addition salts.

The lower alkyl groups in the compounds of Formula III contain 1-6 carbon atoms and include methyl, ethyl, propyl, butyl, pentyl, hexyl, and the corresponding branched chain isomers thereof. The halo variants can be fluoro, chloro, bromo, or iodo substituents.

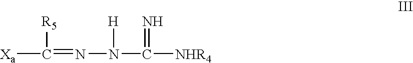

Equivalent to the compounds of Formula III for the purpose of this invention are the biocompatible and pharmaceutically acceptable salts thereof.

Such salts can be derived from a variety of organic and inorganic acids including but not limited to methanesulfonic, hydrochloric, toluenesulfonic, sulfuiric, maleic, acetic and phosphoric acids.

Of the compounds encompassed by Formula III, certain substituents are preferred. For instance, R4 is preferably a methyl group and Xa is preferably a phenyl or substituted phenyl group.

Representative of the compounds of Formula III are:

N-acetyl-2-(phenylmethylene)hydrazinecarboximidamide; 2-(phenylmethylene)hydrazinecarboximidamide; 2-(2,6-dichlorophenylmethylene) hydrazinecarboximidamide pyridoxal guanylhydrazone; pyridoxal phosphate guanylhydrazone; 2-(1-methylethylidene)hydrazinecarboximidamide; pyruvic acid guanylhydrazone; 4-acetamidobenzaldehyde guanylhydrazone; 4-acetamidobenzaldehyde N-acetylguanylhydrazone; acetoacetic acid guanylhydrazone; and the biocompatible and pharmaceutically acceptable salts thereof.

Formula IV comprises a structure wherein R6 is hydrogen or a lower alkyl group, or a phenyl group, optionally substituted by 1-3 halo, amino, hydroxy or lower alkyl groups, R7 is hydrogen, a lower alkyl group, or an amino group and R8 is hydrogen or a lower alkyl group and includes their biocompatible and pharmaceutically acceptable acid addition salts.

The lower alkyl groups in the compounds of Formula IV contain 1-6 carbon atoms and include methyl, ethyl, propyl, butyl, pentyl, hexyl, and the corresponding branched chain isomers thereof. The halo variants can be fluoro, chloro, bromo, or iodo substituents. Where the phenyl ring is substituted, the point or points of substitution may be ortho meta or para to the point of attachment of the phenyl ring to the straight chain of the molecule.

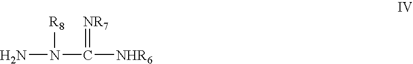

Representative of the compounds of Formula IV are: equival n-butanehydrazonic acid hydrazide; 4-methylbenzamidrazone; N-methylbenzenecarboximidic acid hydrazide; benzenecarboximidic acid 1-methylhydrazide; 3-chlorobenzamidrazone; 4-chlorobenzamidrazone; 2-fluorobenzamidrazone; 3-fluorobenzamidrazone; 4-fluorobenzamidrazone; 2-hydroxybenzamidrazone; 3-hydroxybenzamidrazone, 4-hydroxybenzamidrazone: 2-aminobenzamidrazone; benzenecarbohydrazonic acid hydrazide; benzenecarbohydrazonic acid 1-methylhydrazide; and the biocompatible and pharmaceutically acceptable salts thereof.

Formula V comprises a structure wherein R9 and R10 are independently hydrogen, hydroxy, lower alkyl or lower alkoxy, with the proviso that the “floating” amino group is adjacent to the fixed amino group, and includes their biocompatible and pharmaceutically acceptable acid addition salts.

The lower alkyl groups of the compounds of Formula V contain 1-6 carbon atoms and include methyl, ethyl, propyl, butyl, pentyl, hexyl, and the corresponding branched chain isomers thereof. Likewise, the lower alkoxy groups of the compounds of formula V contain 1-6 carbon atoms and include methoxy, ethoxy, propoxy, butoxy pentoxy, hexoxy, and the corresponding branched chain isomers thereof.

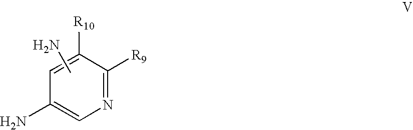

Equivalent to the compounds of Formula V for the purpose of this invention are the biocompatible and pharmaceutically acceptable salts thereof.

Such salts can be derived from a variety of organic and inorganic acids including but not limited to methanesulfonic, hydrochloric, toluenesulfonic, sulfuric, maleic, acetic and phosphoric acids.

Of the compounds encompassed by Formula V, certain substituents are preferred. For instance, when R9 is hydrogen then R10 is preferably also hydrogen. Representative of the compounds of Formula V are: 3,4-diaminopyridine; 2,3-diaminopyridine; 5-methyl-2,3-diaminopyridine; 4-methyl-2,3-diaminopyridine; 6-methyl-2,3-pyridinediamine; 4,6-dimethyl-2,3-pyridinediamine; 6-hydroxy-2,3-diaminopyridine; 6-ethoxy-2,3-diaminopyridine; 6-dimethylamino-2,3-diaminopyridine; diethyl 2-(2,3-diamino-6-pyridyl) malonate; 6 (4-methyl-1-pyperazinyl)-2,3-pyridinediamine; 6-(methylthio)-5 (trifluoromethyl)-2,3-pyridinediamine; 5-(trifluoromethyl)-2,3-pyridinediamine; 6-(2,2,2-trifluorethoxy)-5-(trifluoromethyl)-2,3-pyridinediamine; 6-chloro-5-(trifluoromethyl)-2,3-pyridinediamine; 5-methoxy-6-(methylthio)-2,3-pyridinediamine; 5-bromo-4-methyl-2,3-pyridinediamine; 5-(trifluoromethyl-2,3-pyridinediamine; 6-bromo4-methyl-2,3-pyridinedlamine; 5-bromo-6-methyl-2,3-pyridinediamine; 6-methoxy-3,4-pyridinediamine; 2-methoxy-3,4-pyridinediamine; 5-methyl-3,4-pyridinediamine; 5-methoxy-3,4-pyridinediamine; 5-bromo-3,4-pyridinediamine; 2,3,4-pyridinetriamine; 2,3,5-pyridinetriamine; 4-methyl-2,3,6-pyridinetriamine; 4-(methylthio)-2,3,6-pyridinetriamine; 4-ethoxy-2,3,6-pyridinetriamine; 2,3,6-pyridinetriamine; 3,4,5-pyridinetriamine; 4-methoxy-2,3-pyridinediamine; 5-methoxy-2,3-pyridinediamine; 6-methoxy-2,3-pyridinediamine; and the biocompatible and pharmaceutically acceptable salts thereof.

Formula VI comprises a structure wherein n is 1 or 2, R11 is an amino group or a hydroxyethyl group, and R12 is an amino, a hydroxyalkylamino, a lower alkyl group or a group of the formula alk-Ya wherein alk is a lower alkylene group and Ya is selected from the group consisting of hydroxy, lower alkoxy, lower alkylthio, lower alkylamino and heterocyclic groups containing 4-7 ring members and 1-3 heteroatoms; with the proviso that when R11 is a hydroxyethyl group then R, is an amino group; their biocompatible and pharmaceutically acceptable acid addition salts.

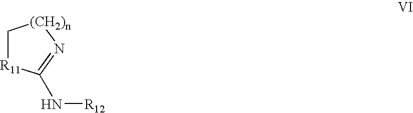

The lower alkyl, lower alkylene and lower alkoxy groups referred to herein contain 1-6 carbon atoms and include methyl, methylene, methoxy, ethyl, ethylene, ethoxy, propyl, propylene, propoxy, butyl, butylene, butoxy, pentyl, pentylene, pentyloxy, hexyl, hexylene, hexyloxy and the corresponding branched chain isomers thereof. The heterocyclic groups referred to herein include 4-7 member rings having at least one and up to 3 heteroatoms therein.

Representative heterocyclic groups are those such as morpholino, piperidino, piperazino, methylpiperazino, and hexamethylenimino.

Equivalent to the compounds of Formula VI for the purpose of this invention are the biocompatible and pharmaceutically acceptable salts thereof.

Such salts can be derived from a variety of organic and inorganic acids including but not limited to, methanesulfonic, hydrochloric, toluenesulfonic, sulfuric, maleic, acetic and phosphoric acids.

Of the compounds encompassed by Formula VI, certain combinations of substituents are preferred. For instance, when R11 is a hydroxyethyl group, then R12 is an amino group. When R11is an amino group, then R12 is preferably a hydroxy lower alkylamino, a lower alkyl group or a group of the formula alk-Y, wherein alk is a lower alkylene group and Y is selected from the group consisting of hydroxy, lower alkoxy, lower alkylthio, lower alkylamino and heterocyclic groups containing 4-7 ring members and 1-3 heteroatoms.

Representative of the compounds of Formula VI are:
1-amino-2-[2-(2-hydroxyethyl)hydrazino]-2-imidazoline; 1-amino-[2-(2-hydroxyethyl)hydrazino]-2-imidazoline; 1-amino-2-(2-hydroxyethylamino)-2-imidazoline; 1-(2-hydroxyethyl)-2-hydrazino-1,4,5,6-tetrahydropyrimidine; 1-(2-hydroxyethyl)2-hydrazino-2-imidazoline; 1-amino-2-([2-(4-morpholino) ethyl]amino)imidazoline; ([2-(4-morpholino)ethyl] amino)imidazoline; 1-amino-2-([3-(4-morpholino)propyl]amino)imidazoline; 1-amino-2-([3-(4-methylpiperazin-1-yl)propyl]-amino)imidazoline; 1-amino-2-([3-(dimethylamino)propyl]amino)imidazoline; 1-amino-2-[(3-ethoxypropyl)amino]imidazoline; 1-amino-2-([3-(1-imidazolyl)propyl]amino)imidazoline; 1-amino-2-(2-methoxyethylamino)-2-imidazoline; (2-methoxyethylamino)-2-imidazoline; 1-amino-2-(3-isopropoxypropylamino)-2-imidazoline; 1-amino-2-(3-methylthiopropylamino)-2-imidazoline; 1-amino-2[3-(1-piperidino)propylamino)imidazoline; 1-amino-2-[2,2-dimethyl-3-(dimethylamino)propylamino]-2-imidazoline; 1-amino-2-(neopentylamino)-2-imidazoline; and the biocompatible and pharmaceutically acceptable salts thereof.

Formula VII comprises a structure wherein R13 is a hydrogen or an amino group, R14 and R15 are independently an amino group, a hydrazino group, a lower alkyl group, or an aryl group with the proviso that one of R13, R14 and R15 must be an amino or a hydrazino group, and includes their biologically or pharmaceutically acceptable acid or alkali addition salts.

The lower alkyl groups referred to above preferably contain 1-6 carbon atoms and include methyl, ethyl, propyl, butyl, pentyl, hexyl, and the corresponding branched-chain isomers thereof.

The aryl groups encompassed by the Formula VII are those containing 6-10 carbon atoms, such as phenyl and lower alkyl substituted-phenyl, e. g. tolyl and xylyl, and phenyl substituted by 1-2 halo, hydroxy or lower alkoxy groups.

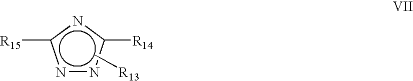

The halo atoms in the Formula VII may be fluoro, chloro, bromo, or iodo. The lower alkoxy groups contain 1-6, and preferably 1-3, carbon atoms and are illustrated by methoxy, ethoxy, n-propoxy, isopropoxy and the like.

For the purposes of this invention equivalent to the compounds of Formula VII are the biologically and pharmaceutically acceptable acid addition salts thereof. Such acid addition salts may be derived from a variety of organic and inorganic acids such as sulfuric, phosphoric, hydrochloric, hydrobromic, sulfamic, citric, lactic, maleic, succinic, tartaric, cinnamic, acetic, benzoic, gluconic, ascorbic and related acids.

Of the compounds encompassed by Formula VII, certain combinations of substituents are preferred. For instance, when R13 is hydrogen, then R14 is preferably an amino group. When R14 is a hydrazino group, then R is preferably an amino group.

Representative of the compounds of Formula VII are:
3,4-diamino-5-methyl-1,2,4-triazole; 3,5-dimethyl4H-1,2,4-triazol-4-amine; 4-triazol-4-amine; 4-triazol-4-amine; 4-triazol-4-amine; 2,4-triazole-3,4-diamine; 5-(1-ethylpropyl)4H-1,2,4-triazole-3,4-diamine; 5-isopropyl-4H-1,2,4-triazole-3,4-diamine; 5-cyclohexyl-4H-1,2,4-triazole-3,4-diamine; 5-methyl-4H-1,2,4-triazole-3,4-diamine; 5-phenyl-4H-1,2,4-triazole-3,4-diamine; 5-propyl-4H-1,2,4-triazole-3,4-diamine; 5-cyclohexyl-4H-1,2,4-triazole-3,4-diamine.

Formula VIII comprises a structure wherein R16 is hydrogen or an amino group, R17 is an amino group or a guanidino group when R16 is hydrogen, or R17 is an amino group when R16 is an amino group, R18 and R19 are independently hydrogen, hydroxy, a lower alkyl group, a lower alkoxy group, or an aryl group, and includes their biologically or pharmaceutically acceptable acid or alkali addition salts.

The lower alkyl groups in the compounds of Formula VIII preferably contain 1-6 carbon atoms and include methyl, ethyl, propyl, butyl, pentyl, hexyl, and the corresponding branched chain isomers thereof. The lower alkoxy groups likewise contain 1-6, and preferably 1-3, carbon atoms, and are illustrated by methoxy, ethoxy, n-propoxy, isopropoxy and the like.

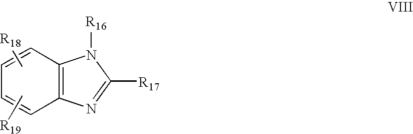

The aryl groups encompassed by the above formula are those containing 6-10 carbon atoms, such as phenyl and lower alkyl substituted-phenyl, e. g., tolyl and xylyl, and phenyl substituted by 1-2 halo, hydroxy or lower alkoxy groups.

The halo atoms in the above Formula VIII may be fluoro, chloro, bromo or iodo.

The biologically or pharmaceutically acceptable salts of the compounds of Formula VIII are those tolerated by the mammalian body and include acid addition salts derived from a variety of organic and inorganic acids such as sulfuric, phosphoric, hydrochloric, sulfamic, citric, lactic, maleic, succinic, tartaric, cinnamic, acetic, benzoic, gluconic, ascorbic and related acids. Of the compounds encompassed by Formula VIII, certain substituents are preferred. For instance, the compounds wherein R, is an amino group are preferred group.

Representative of the compounds of Formula VIII are:
2-guanidinobenzimidazole; 1,2diaminobenzimidazole; 1,2-diaminobenzimidazole hydrochloride; 5-bromo-2-guanidinobenzimidazole; 5-methoxy-2-guanidinobenzimidazole; 5-methylbenzimidazole-1,2-diamine; 5-chlorobenzimidazole-1,2-diamine; and 2,5-diaminobenzimidazole;

Formula IX, comprising R20-CH—(NHR21)—COOH(IX), is a structural formula wherein R20 is selected from the group consisting of hydrogen; lower alkyl, optionally substituted by one or two hydroxyl, thiol, phenyl, hydroxyphenyl, lower alkylthiol, carboxy, aminocarboxy or amino groups and R21, is selected from the group of hydrogen and an acyl group; and their biocompatible and pharmaceutically acceptable acid addition salts.
R₂₀—CH—(NHR₂₁)—CO₂H IX
The lower alkyl groups of the compounds of Formula IX contain 1-6 carbon atoms and include methyl, ethyl, propyl, butyl, pentyl, hexyl and the corresponding branched chain isomers thereof.

The acyl groups referred to herein are residues of lower alkyl, aryl and heteroaryl carboxylic acids containing 2-10 carbon atoms. They are typified by acetyl, propionyl, butanoyl, valeryl, hexanoyl and the corresponding higher chain and branched chain analogs thereof. The acyl radicals may also contain one or more double bonds and/or an additional acid functional group e. g., glutaryl or succinyl.

The amino acids utilized herein can possess either the L & D; stereochemical configuration or be utilized as mixtures thereof. However, the L-configuration is preferred.

Equivalent to the compounds of Formula IX for the purposes of this invention are the biocompatible and pharmaceutically acceptable salts thereof. Such salts can be derived from a variety of inorganic and organic acids such as methanesulfonic, hydrochloric, toluenesulfonic, sulfuric, maleic, acetic, phosphoric and related acids.

Representative compounds of the compounds of Formula IX are: 5 lysine; 2,3-diaminosuccinic acids; cysteine and the biocompatible and pharmaceutically acceptable salts thereof.

Formula X comprises a structure wherein R22 and R23 are independently hydrogen, an amino group or a mono-or di-amino lower alkyl group, R24 and R25 are independently hydrogen, a lower alkyl group, an aryl group, or an acyl group with the proviso one of R22 and R23 must be an amino group or an mono- or diamino lower alkyl group, and includes their biologically or pharmaceutically acceptable acid or alkali addition salts.

The lower alkyl groups of the compounds of Formula X contain 1-6 carbon atoms and include methyl, ethyl, propyl, butyl, pentyl, hexyl, and the corresponding branched-chain isomers thereof. The mono-or di-amino alkyl groups are lower alkyl groups substituted in the chain by one or two amino groups.

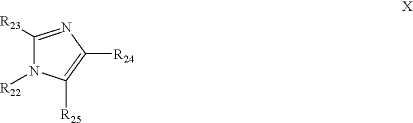

The aryl groups referred to herein encompass those containing 6-10 carbon atoms, such as phenyl and lower alkyl substituted-phenyl, e. g., tolyl and xylyl, and phenyl substituted by 1-2 halo, hydroxy and lower alkoxy groups. The acyl groups referred to herein are residues of lower alkyl, aryl and heteroaryl carboxylic acids containing 2-10 carbon atoms. They are typified by acetyl, propionyl, butanoyl, valeryl, hexanoyl and the corresponding higher chain and branched chain analogs thereof. The acyl radicals may also contain one or more double bonds and/or an additional acid functional group, e. g., glutaryl or succinyl.

The heteroaryl groups referred to above encompass aromatic heterocyclic groups containing 3-6 carbon atoms and one or more heteroatoms such as oxygen, nitrogen or sulfur.

The halo atoms in the above Formula X may be fluoro, chloro, bromo and iodo. The lower alkoxy groups contain 1-6, and preferably 1-3, carbon atoms and are illustrated by methoxy, ethoxy, propoxy, isopropoxy and the like.

The term biologically or phamiaceutically acceptable salts refers to salts which are tolerated by the mammalian body and are exemplified by acid addition salts derived from a variety of organic and inorganic acids such as sulfnric, phosphoric, hydrochloric hydrobromic, hydroiodic, sulfamic, citric, lactic, maleic, succinic, tartaric, cinnamic, acetic, benzoic, gluconic, ascorbic and related acids.

Of the compounds encompassed by Formula X, certain combinations of substituents are preferred. For instance, when R22 and R23 are both amino groups, then R24 and R25 are preferably both hydrogen atoms. When R22 or R23 is amino group and one of R24 or R25 is an aryl group, the other of R24 and R25 is preferably hydrogen.

Representative compounds of Formula X are: 1,2-diamino-4-phenyl[1H]imidazole; 1,2-diaminoimidazole; 1-(2,3-diaminopropyl)imidazole trihydrochloride; 4-(4-bromophenyl)imidazole-1,2-diamine; 4-(4-chlorophenyl)imidazole-1,2-diamine; 4-(4-hexylphenyl)imidazole-1,2-diamine; 4-(4-methoxyphenyl)imidazole-1,2-diamine; 4-phenyl-5-propylimidazole-1 ,2-diamine; 1 ,2-diamino-4-methylimidazole; 1,2-diamino-4,5-dimethylimidazole; and 1,2-diamino4-methyl-5-acetylimidazole.

Formula XI comprises a structure wherein R26 is a hydroxy, lower alkoxy, amino, amino lower alkoxy, mono-lower alkylamino lower alkoxy, di-lower alkylamino lower alkoxy or hydrazino group, or a group of the formula—N R29 R30, wherein R29 is hydrogen or lower alkyl, and R30 is an alkyl group of 1-20 carbon atoms, an aryl group, a hydroxy lower alkyl group, a carboxy lower alkyl group, cyclo lower alkyl group or a heterocyclic group containing 4-7 ring members and 1-3 heteroatoms; or R29 and R30 together with the nitrogen form a morpholino, piperidinyl, or piperazinyl group; or when R29 is hydrogen, then R30 can also be a hydroxy group; R27 is 0-3 amino or nitro groups, and/or a hydrazino group, a hydrazinosulfonyl group, a hydroxyethylamino or an amidino group; R28 is hydrogen or one or two fluoro, hydroxy, lower alkoxy, carboxy, lower alkylamino, di-lower alkylamino or a hydroxy lower alkylamino groups; with the proviso that when R26 is hydroxy or lower alkoxy, then R27 is a non-hydrogen substituent; with the further proviso that when R26 is hydrazino, then there must be at least two non-hydrogen substituents on the phenyl ring; and with the further proviso that when R28 is hydrogen, then R30 can also be an aminoirnino, guanidyl; aminoguanidinyl or diaminoguanidyl group, and includes their pharmaceutically acceptable salts and hydrates.

The lower alkyl groups of the compounds of Formula Xi contain 1-6 carbon atoms and include methyl, ethyl, propyl, butyl, pentyl, hexyl, and the corresponding branched-chain isomers thereof. The cycloalkyl groups contain 4-7 carbon atoms and are exemplified by groups such as cyclobutyl, cyclopentyl, cyclohexyl, 4-methylcyclohexyl and cycloheptyl groups.

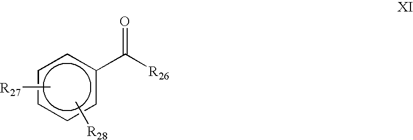

The heterocyclic groups of the compounds of Formula XI include 4-7 membered rings having at least one and up to 3 heteroatoms, e. g., oxygen, nitrogen, or sulfur, therein, and including various degrees of unsaturation.

Representatives of such heterocyclic groups are those such as morpholino, piperidino, homopiperidino, piperazino, methylpiperazino, hexamethylenimino, pyridyl, methylpyridyl, imidazolyl, pyrrolidinyl, 2,6-dimethylmorpholino, furfural, 1,2,4-triazoylyl, thiazolyl, thiazolinyl, methylthiazolyl, and the like.

Equivalent to the compounds of Formula XI for the purposes of this invention are the biocompatible and pharmaceutically acceptable salts and hydrates thereof Such salts can be derived from a variety of organic and inorganic acids, including, but not limited to, methanesulfonic, hydrochloric, hydrobromic, hydroiodic, toluenesulfonic, sulfuric, maleic, acetic and phosphoric acids.

When the compounds of Formula XI contain one or more asymmetric carbon atoms, mixtures of enantiomers, as well as the pure (R) or (S) enantiomeric form can be utilized in the practice of this invention.

In addition, compounds having a 3,4-diamino- or 2,3-diamino-5-fluoro substituent pattern on the phenyl ring are highly preferred.

Representative compounds of formula XI of the present invention are:
4-(cyclohexylamino-carbonyl)-o-phenylene diamine hydrochloride; 3,4-diaminobenzhydrazide; 4-(n-butylamino-carbonyl)-o-phenylene-diamine dihydrochloride; 4-(ethylamino-carbonyl)-o-phenylene-diamine dihydrochloride; 4-carbamoyl-o-phenyiene diamine hydrochloride; 4-(morpholino-carbonyl)-o-phenylene-diamine hydrochloride; 4-[(4-morpholino)hydrazino-carbonyl]-o-phenylenediamine; 4-(l -piperidinylamino-carbonyl)-o-phenylenediamine dihydrochloride; 2,4-diamino-3-hydroxybenzoic acid; 4,5-diamino-2-hydroxybenzoic acid; 3,4-diaminobenzamide; 3,4-diaminobenzhydrazide; 3,4-diamino-N,N-bis(1-methylethyl)benzamide; 3,4-diamino-N,N-diethylbenzamide; 3,4-diamino-N,N-dipropylbenzamide; 3,4-diamino-N-(2-furanylmethyl)benzamide 3,4-diamino-N-(2-methylpropyl)benzamide; benzamide; 3,4-diamino-N-(5-methyl-2-thiazolyl)benzamide; 3,4-diamino-N-(6-methoxy-2-benzothiazolyl)benzamide; 3,4-diamino-N-(6-methoxy-8-quinolinyl)benzamide; 3,4-diamino-N-(6-methyl-2-pyridinyl)benzamide; 3,4-diamino-N-(1H-benzimidazol-2-yl)benzamide; 3,4-diamino-N-(2-pyridinyl)benzamide; 3,4-diamino-N-(2-thiazolyl)benzamide; 3,4-diamino-N-(4-pyridinyl)benzamide; 3,4-diamino-N-[9H-pyrido(3,4-b)indol-6-yl]benzamide 3,4-diamino-N-butylbenzamide; 3,4-diamino-N-cyclohexylbenzamide; 3,4-diamino-N-cyclopentylbenzamide; 3,4-diamino-N-decylbenzamide; 3,4-diamino-N-dodecylbenzamide; 3,4-diarnino-N-methylbenzamide; 3,4-diamino-N-octylbenzamide; 3,4-diamino-N-pentylbenzamide; 3,4-diamino-N-phenylbenzamide; 4-(diethylamino-carbonyl)-o-phenylene diamine; 4-(tert-butylamino-carbonyl)-o-phenylene diamine; 4-isobutylamino-carbonyl)-o-phenylene diamine; 4-(neopentylamino-carbonyl)-o-phenylene diamine; 4-(dipropylamino-carbonyl)-o-phenylene diamine; 4-(n-hexylamino-carbonyl)-o-phenylene diamine; 4-(n-decylamino-carbonyl)-o-phenylene diamine; 4-(n-dodecylamino-carbonyl)-o-phenylene diamine; 4-(1-hexadecylamino-carbonyl)-o-phenylene diamine; 4-(octadecylamino-carbonyl)-o-phenylene diamine; 4-(hydroxylamino-carbonyl)-o-phenylene diamine; 4-(2-hydroxyethylamino-carbonyl)-o-phenylene; 4-[(2-hydroxyethylamino)ethylamino-carbonyl]-o-phenylene diamine; 4-[(2-hydroxyethyloxy)ethylamino-carbonyl]-o-phenylene diamine; 4-(6-hydroxyhexylamino-carbonyl)-o-phenylene diamine; 4-(3-ethoxypropylamino-carbonyl)-o-phenylene diamine; 4-(3-isopropoxypropylamino-carbonyl)-o-phenylene diamine; 4-(3-dimethylaminopropylamino-carbonyl)-o-phenylene diamine; 4-[4-(2-aminoethyl)morpholino-carbonyl]-o-phenylene diamine; 4-[4-(3-aminopropyl) morpholino-carbonyl]-o-phenylene diamine; 4-N-(3-aminopropyl)pyrrolidino-carbonyl]-o-phenylene diamine; 4-[3-(N-piperidino)propylamino-carbonyl]-o-phenylene diamine; 4-[3-(4-methylpiperazinyl)propylamino-carbonyl]-o-phenylene diamine; 4-(3-imidazoylpropylamino-carbonyl)-o-phenylene diamine; 4-(3-phenylpropylamino-carbonyl)-o-phenylenediamine; 4-[2-(N,N-diethylamino) ethylamino-carbonyl]-o-phenylene diamine; 4-(imidazolylamino-carbonyl)-o-phenylene diamine; 4-(pyrrolidinyl-carbonyl)-o-phenylene diamine; 4-(piperidino-carbonyl)-o-phenylene diamine; 4-(1-methylpiperazinyl-carbonyl)-o-phenylene diamine; 4-(2,6dimethylmorpholino-carbonyl)-o-phenylenediamine; 4-(pyrrolidin-1-ylamino-carbonyl)-o-phenylene diamine; 4-(homopiperidin-1-ylamino-carbonyl)-o-phenylene diamine; 4-(4-methylpiperazine-1-ylamino-carbonyl)-o-phenylene diamine; 4-(1,2,4-triazol-1-ylamino-carbonyl)-o-phenylene diamine; 4-(guanidinyl-carbonyl)-o-phenylene diamine; 4-(guanidinylamino-carbonyl)-o-phenylene diamine; 4-aminoguanidinylamino-carbonyl)-o-phenylene diamine; 4-(diaminoguanidinylamino-carbonyl)-o-phenylene diamine; 3,4-aminosalicylic acid 4-guanidinobenzoic acid; 3,4-diaminobenzohydroxamic acid; 3,4,5-triaminobenzoic acid; 2,3-diamino-5-fluoro-benzoic acid; and 3,4-diaminobenzoic acid; and their pharmaceutically acceptable salts and hydrates.

Formula XII comprises a structure wherein R3 1, is hydrogen, a lower alkyl or hydroxy group; R32 is hydrogen, hydroxy lower alkyl, a lower alkoxy group, a lower alkyl group, or an aryl group; R33 is hydrogen or an amino group; and their biologically or pharmaceutically acceptable acid addition salts.

The lower alkyl groups of the compounds of Formula XII contain 1-6 carbon atoms and include methyl, ethyl, propyl, butyl, pentyl, hexyl, and the corresponding branched-chain isomers thereof. Likewise, the lower alkoxy groups contain 1-6, and preferably 1-3, carbon atoms and include methoxy, ethoxy, isopropoxy, propoxy, and the like. The hydroxy lower alkyl groups include primary, secondary and tertiary alcohol substituent patterns.

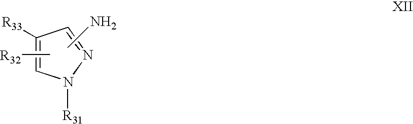

The aryl groups of the compounds of Formula XII encompass those containing 6-10 carbon atoms, such as phenyl and lower alkyl substituted-phenyl, e. g., tolyl and xylyl, and phenyl substituted by 1-2 halo, hydroxy and lower alkoxy groups.The halo atoms in the above Formula XII may be fluoro, chloro, bromo, and iodo.

The term ‘biologically or pharmaceutically acceptable salts” refers to salts which are tolerated by the mammalian body and are exemplified by acid addition salts derived from a variety of organic and inorganic acids such as sulfuric, phosphoric, hydrochloric hydrobromic, hydroiodic, sulfamic, citric, lactic, maleic, succinic, tartaric, cinnamic, acetic, benzoic, gluconic, ascorbic and related acids.

Of the compounds encompassed by Formula XII, certain substituents are preferred. For instance, the compounds wherein R32 is hydroxy and R33 is an amino group are preferred.

Representative of the compounds of Formula XII include, but should not be limited to:
3,4-diaminopyrazole; 3,4-diamino-5-hydroxypyrazole; 3,4-diamino-5-methylpyrazole 3,4-diamino-5-methoxypyrazole; 3,4-diamino-5-phenylpyrazole; 1-methyl-3-hydroxy-4,5-diaminopyrazole; 1-(2-hydroxyethyl)-3-hydroxy-4,5-diaminopyrazole; 1-(2-hydroxyethyl)-3-phenyl-4,5-diaminopyrazole; 1-(2-hydroxyethyl)-3-methyl-4,5-diaminopyrazole; 1-(2-hydroxyethyl)-4,5-diaminopyrazole; 1-(2-hydroxypropyl)-3-hydroxy-4,5-diaminopyrazole; 3-amino-5-hydroxypyrazole; and 1-(2-hydroxy-2-methylpropyl)-3-hydroxy-4,5-diaminopyrazole; and their biologically and pharmaceutically acceptable acid addition salts.

Formula XIII comprises a structure where n=1-6, wherein X is —NR1-, —S(O)—, —S(O)2-, or —O—, R1 being selected from the group consisting of H, linear chain alkyl group (C1-C6) and branched chain alkyl group (C1-C6). Y═—N—, —NH—, or —O— and Z is selected from the group consisting of H, linear chain alkyl group (C1-C6) and branched chain alkyl group (C1-C6).

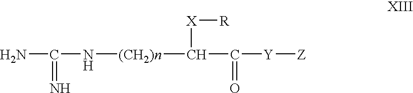

For Formula XIV, wherein R37 is a lower alkyl group, or a group of the formula NR41NR42, wherein R41 is hydrogen and R42 is a lower alkyl group or a hydroxy (lower) alkyl group; or R41 and R42 together with the nitrogen atom are a heterocyclic group containing 4-6 carbon atoms and, in addition to the nitrogen atom, 0-1 oxygen, nitrogen or sulfur atoms; R38 is hydrogen or an amino group; R39 is hydrogen or an amino group; R40 is hydrogen or a lower alkyl group; with the proviso that at least one of R38, R39, and R40 is other than hydrogen; and with the further proviso that R37 and R38 cannot both be amino groups; and their pharmaceutically acceptable acid addition salts.

The lower alkyl groups of the compounds of Formula XIV contain 1-6 carbon atoms and include methyl, ethyl, propyl, butyl, pentyl, hexyl, and the corresponding branched-chain isomers thereof.

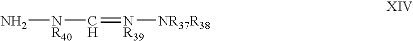

The heterocyclic groups formed by the NR41 R42 group are 4-7 membered rings having at 0-1 additional heteroatoms, e. g., oxygen, nitrogen, or sulfur, therein, and including various degrees of unsaturation. Representatives of such heterocyclic groups are those such as morpholino, piperidino, hexahydroazepino, piperazino, methylpiperazino, hexamethylenimino, pyridyl, methylpyridyl, imidazolyl, pyrrolidinyl, 2,6-dimethylmorpholino, 1,2,4-triazoylyl, thiazolyl, thiazolinyl, and the like.

Equivalent to the compounds of Formula XIV for the purposes of this invention are the biocompatible and pharmaceutically acceptable salts thereof. Such salts can be derived from a variety of organic and inorganic acids, including, but not limited to, methanesulfonic, hydrochloric, hydrobromic, hydroiodic, toluenesulfonic, sulfuric, maleic, acetic and phosphoric acids.

When the compounds of Formula XIV contain one or more asymmetric carbon atoms, mixtures of enantiomers, as well as the pure (R) or (S) enantiomeric form can be utilized in the practice of this invention.

Of the compounds encompassed by Formula XIV, certain combinations of substituents are preferred. For instance, compounds wherein R37 is a heterocyclic group, and particularly a morpholino or a hexahydroazepino group, are highly preferred.

Representative of the compounds of Formula XIV are: 2-(2-hydroxy-2-methylpropyl) hydrazinecarboximidic hydrazide; N-(4-morpholino)hydrazinecarboximidamide; 1-methyl-N-(4-morpholino)hydrazinecarboximidamide; 1-methyl-N-(4-piperidino)hydrazinecarboximidamide; 1-(N-hexahydroazepino) hydrazinecarboximidamide; N,N-dimethylcarbonimidic dihydrazide; 1-methylcarbonimidic dihydrazide; 2-(2-hydroxy-2-methylpropyl) carbohydrazonic dihydrazide; and N-ethylcarbonimidic dihydrazide.

Formula XV is a structure comprising (R43HN═) CR44-W-CR45 (═NHR43) (XV); wherein R43 is pyridyl, phenyl or a carboxylic acid substituted phenyl group of the formula; wherein R46 is hydrogen, lower alkyl or a water-solubilizing ester moiety; W is a carbon-carbon bond or an alkylene group of 1-3 carbon atoms, R44 is a lower alkyl, aryl, or heteroaryl group and R45 is hydrogen, a lower alkyl, aryl or heteroaryl group; and it includes their biologically or pharmaceutically acceptable acid addition salts.

The lower alkyl groups of the compounds of Formula XV preferably contain 1-6 carbon atoms and include methyl, ethyl, propyl, butyl, pentyl, hexyl, and the corresponding branched-chain isomers thereof. These groups are optionally substituted by one or more halo, hydroxy, amino or lower alkylamino groups.

The alkylene groups of the compounds of Formula XV likewise can be straight or branched chain, and are thus exemplified by ethylene, propylene, butylene, pentylene, hexylene, and their corresponding branched chain isomers.

In the R groups which are a carboxylic acid substituted phenyl group of the formula: 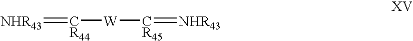
wherein R44 is hydrogen, lower alkyl or a water-solubilizing ester moiety, the water solubilizing ester moiety can be selected from a variety of such esters known in the art. Typically, these esters are derived from dialkylene or trialkylene glycols or ethers thereof, dihydroxyalkyl groups, arylalkyl group, e. g., nitrophenylalkyl and pyridylalkyl groups, and carboxylic acid esters and phosphoric acid esters of hydroxy and carboxy-substituted alkyl groups. Particularly preferred water solubilizing ester moieties are those derived from 2,3-dihydroxypropane, and 2-hydroxyethylphosphate.

The aryl groups encompassed by the above Formula XV are those containing 6-10 carbon atoms, such as phenyl and lower alkyl substituted-phenyl, e. g., tolyl and xylyl, and are optionally substituted by 1-2 halo, nitro, hydroxy or lower alkoxy groups.

Where the possibility exists for substitution of a phenyl or aryl ring, the position of the substituents may be ortho, meta, or para to the point of attachment of the phenyl or aryl ring to the nitrogen of the hydrazine group.

The halo atoms in the above Formula XV may be fluoro, chloro, bromo or iodo. The lower alkoxy groups contain 1-6, and preferably 1-3, carbon atoms and are illustrated by methoxy, ethoxy, n-propoxy, isopropoxy and the like.

The heteroaryl groups in the above Formula XV contain 1-2 heteroatoms, i. e., nitrogen, oxygen or sulfur, and are exemplified by furyl, pyrrolinyl, pyridyl, pyrimidinyl, thienyl, quinolyl, and the corresponding alkyl substituted compounds.

For the purposes of this invention equivalent to the compounds of Formula XV are the biologically and pharmaceutically acceptable acid addition salts thereof. Such acid addition salts may be derived from a variety of organic and inorganic acids such as sulfuric, phosphoric, hydrochloric, hydrobromic, sulfamic, citric, lactic, maleic, succinic, tartaric, cinnamic, acetic, benzoic, gluconic, ascorbic, methanesulfonic and related acids.

Of the compounds encompassed by Formula XV, certain substituents are preferred. For instance, the compounds wherein W is a carbon-carbon bond, R44 is a methyl group and R45 is hydrogen are preferred.

Representative of the compounds of Formula XV are: methylglyoxal bis-(2-hydrazino-benzoic acid)hydrazone; methylglyoxal bis-(dimethyl-2-hydrazinobenzoate)hydrazone; methylglyoxal bis-(phenylhydrazine)hydrazone; methyl glyoxal bis-(dimethyl-2-hydrazinobenzoate)hydrazone; methylglyoxal bis-(4-hydrazinobenzoic acid)hydrazone; methylglyoxal bis-(dimethyl-4-hydrazinobenzoate) hydrazone; methylglyoxal bis-(2-pyridyl)hydrazone; methylglyoxal bis-(diethyleneglycol methylether-2-hydrazinobenzoate)hydrazone; methylglyoxal bis-[1-(2,3-dihydroxypropane)-2-hydrazinebenzoatehydrazone; methyl glyoxal bis-[1-(2-hydroxyethane)-2-hydrazinobenzoate]hydrazone; methylglyoxal bis-[(1-hydroxymethyl-1-acetoxy))-2-hydrazino-2-benzoate]hydrazone; methylglyoxal bis-[(4-nitrophenyl)-2-hydrazinobenzoate]hydrazone; methylglyoxal bis-[(4-methylpyridyl)-2-hydrazinobenzoate]hydrazone; methylglyoxal bis-(triethylene glycol 2-hydrazinobenzoate)hydrazone; and methylglyoxal bis-(2-hydroxyethylphosphate-2-hydrazinebenzoate)hydrazone.

Formula XVI comprises a structure wherein R47 and R48 are each hydrogen or, together, are an alkylene group of 2-3 carbon atoms, or, when R47 is hydrogen, then R48 can be a group of the formula alk-N—R50 R51, wherein alk is a straight or branched chain alkylene group of 1-8 carbon atoms, and R50 and R51 are independently each a lower alkyl group of 1-6 carbon atoms, or together with the nitrogen atom form a morpholino, piperdinyl or methylpiperazinyl group; R49 is hydrogen, or when R47 and R48 are together an alkylene group of 2-3 carbon atoms, a hydroxyethyl group; W is a carbon-carbon bond or an alkylene group of 1-3 carbon atoms, and R52 is a lower alkyl, aryl, or heteroaryl group and R53 is hydrogen, a lower alkyl, aryl or heteroaryl group; with the proviso that when W is a carbon-carbon bond, then R52 and R53 together can also be a 1,4-butylene group; or W is a 1,2-, 1,3-, or 1,4-phenylene group, optionally substituted by one or two lower alkyl or amino groups, a 2,3-naphthylene group; a 2,5-thiophenylene group; or a 2,6-pyridylene group; and R52 and R53 are both hydrogen or both are lower alkyl groups; or W is an ethylene group and R52 and R53 together are an ethylene group; or W is an ethenylene group and R52 and R53 together are an ethenylene group; or W is a methylene group and R52 and R53 together are a group of the formula═C (—CH3)—N—(H3C—) C═ or —C—W—C— and R52 and R53 together form a bicyclo-(3,3,1)-nonane or a bicyclo-3,3,1-octane group and R47 and R48 are together an alkylene group of 2-3 carbon atoms and R49 is hydrogen; and their biologically or pharmaceutically acceptable acid addition salts.

The lower alkyl groups of the compounds of Formula XVI preferably contain 1-6 carbon atoms and include methyl, ethyl, propyl, butyl, pentyl, hexyl, and the corresponding branched-chain isomers thereof. These groups are optionally substituted by one or more halo hydroxy, amino or lower alkylamino groups.

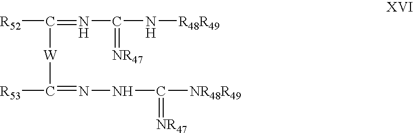

The alkylene groups of the compounds of Formula XVI likewise can be straight or branched chain, and are thus exemplified by ethylene, propylene, butylene, pentylene, hexylene, and their corresponding branched chain isomers.

The aryl groups encompassed by the above Formula XVI are those containing 6-10 carbon atoms, such as phenyl and lower alkyl substituted-phenyl, e. g. tolyl and xylyl, and are optionally substituted by 1-2 halo, hydroxy or lower alkoxy groups.

The halo atoms in the above Formula XVI may be fluoro, chloro, bromo or iodo. The lower alkoxy groups contain 1-6, and preferably 1-3, carbon atoms and are illustrated by methoxy, ethoxy, n-propoxy, isopropoxy and the like.

The heteroaryl groups in the above Formula XVI contain 1-2 heteroatoms, i. e. nitrogen, oxygen or sulfur, and are exemplified by be furyl, pyrrolinyl, pyridyl, pyrimidinyl, thienyl, quinolyl, and the corresponding alkyl substituted compounds.

For the purposes of this invention equivalent to the compounds of Formula XVI are the biologically and pharmaceutically acceptable acid addition salts thereof. Such acid addition salts may be derived from a variety of organic an inorganic acids such as sulfuric, phosphoric, hydrochloric, hydrobromic, sulfamic, citric, lactic, maleic, succinic, tartaric, cinnamic, acetic, benzoic, gluconic, ascorbic, methanesulfonic and related acids.

Of the compounds encompassed by Formula XVI, certain substituents are preferred. For instance, the compounds wherein R48 and R49 are together an alkylene group of 2-3 carbon atoms are preferred. The compounds wherein R52 and R53 together are a butylene, ethylene, or an ethenylene group and those wherein R52 and R53 are both methyl or furyl groups are also highly preferred.

Representative of the compounds of Formula XVI are: methyl glyoxal bis guanylhydrazone); methyl glyoxal bis(2-hydrazino-2-imidazoline-hydrazone);terephthaldicarboxaldehyde bis(2-hydrazino-2-imidazoline hydrazone); terephaldicarboxaldehyde bis(guanylhydrazone); phenylglyoxal bis(2-hydrazino-2-imidazoline hydrazone); furylglyoxal bis(2-hydrazino-2-imidazoline hydrazone); methyl glyoxal bis (1-(2-hydroxyethyl)-2-hydrazino-2-imidazoline hydrazone); methyl glyoxal bis(1-(2-hydroxyethyl)-2-hydrazino-1,4,5,6-tetrahydropyrimidine hydrazone); phenyl glyoxal bis (guanylhydrazone); phenyl glyoxal bis(1-(2-hydroxyethyl)-2-hydrazino-2-imidazoline hydrazone); furyl glyoxal bis(1-(2-hydroxyethyl)-2-hydrazino-2-imidazoline hydrazone); phenyl glyoxal bis(l-(2-hydroxyethyl)-2-hydrazino-1,4,5,6-tetrahydropyrimidine hydrazone); furyl glyoxal bis(1-(2-hydroxyethyl)-2-hydrazino-1,4,5,6-tetrahydropyrimidine hydrazone); 2,3-butanedione bis (2-hydrazino-2-imidazoline hydrazone); 1,4-cyclohexanedione bis(2-hydrazino-2-imidazoline hydrazone); o-phthalic dicarboxaldehyde bis(2-hyd carboximidamide hydrazone); furyglyoxal bis(guanyl hydrazone)dihydrochloride dihydrate; 2,3-pentanedione bis(2-tetrahydropyrimidine)hydrazone dihydrobromide; 1,2-cyclohexanedione bis(2-tetrahydropyrimidine)hydrazone dihydrobromide; 2,3-hexanedione bis(2-tetrahydropyrimidine)hydrazone dihydrobromide; 1,3-diacetyl bis(2-tetrahydropyrimidine)hydrazone dihydrobromide; 2,3-butanedione bis(2-tetrahydropyrimidine)hydrazone dihydrobromide; 2,6-diacetylpyridine-bis-(2-hydrazino-2-imidazoline hydrazone)dihydrobromide; 2,6-diacetylpyridine-bis-(guanyl hydrazone)dihydrochloride; 2,6-pyridine dicarboxaldehyde-bis-(2-hydrazino-2-imidazoline hydrazone)dihydrobromide trihydrate); 2,6-pyridine dicarboxaldehyde-bis(guanyl hydrazone)dihydrochloride; 1,4-diacetyl benzene-bis-(2-hydrazino-2-imidazoline hydrazone)dihydrobromide dihydrate; 1,3-diacetyl benzene-bis-(2-hydrazino-2-imidazoline)hydrazone dihydrobromide; 1,3-diacetyl benzene-bis(guanyl)-hydrazone dihydrochloride; isophthalaldehyde-bis-(2-hydrazino-2-imidazoline)hydrazone dihydrobromide; isophthalaldehyde-bis-(guanyl)hydrazone dihydrochloride; 2,6-diacetylaniline bis-(guanyl)hydrazone dihydrochloride; 2,6-diacetyl aniline bis-(2-hydrazino-2-imidazoline)hydrazone dihydrobromide; 2,5-diacetylthiophene bis(guanyl)hydrazone dihydrochloride; 2,5-diacetylthiophene bis-(2-hydrazino-2-imidazoline)hydrazone dihydrobromide; 1,4-cyclohexanedione bis(2-tetrahydropyrimidine)hydrazone dihydrobromide; 3,4-hexanedione bis(2-tetrahydropyrimidine)hydrazone dihydrobromide; methylglyoxal-bis-(4-amino-3-hydrazino-1 ,2,4-triazole)hydrazone dihydrochloride; methylglyoxal-bis-(4-amino-3-hydrazino-5-methyl-1,2,4-triazole)hydrazone dihydrochloride; 2,3-pentanedione-bis-(2-hydrazino-3-imidazoline)hydrazone dihydrobromide; 2,3-hexanedione-bis-(2-hydrazino-2-imidazoline)hydrazone dihydrobromide; 3-ethyl-2,4-pentane dione-bis-(2-hydrazino-2-imidazoline)hydrazone dihydrobromide; methylglyoxal-bis-(4-amino-3-hydrazino-5-ethyl-1,2,4-triazole)hydrazone dihydrochloride; methylglyoxal-bis-(4-amino-3-hydrazino-5-isopropyl-1,2,4-triazole)hydrazone dihydrochloride; methyl glyoxal-bis-(4-amino-3-hydrazino-5-cyclopropyl-1 ,2,4-triazole)hydrazone dihydrochlorimethylglyoxal-bis-(4-amino-3-hydrazino-5-cyclobutyl-1 ,2,4-triazole) hydrazone dihydrochloride; 1,3-cyclohexanedione-bis-(2-hydrazino-2-imidazoline) hydrazone dihydrobromide; 6-dimethyl pyridine bis(guanyl)hydrazone dihydrochloride; 3,5-diacetyl-1,4-dihydro-2,6-dimethylpyridine bis-(2-hydrazino-2-imidazoline hydrazone dihydrobromide; bicyclo-(3,3,1)nonane-3,7-dione bis-(2-hydrazino-2-imidazoline)hydrazone dihydrobromide; and cis-bicyclo-(3,3,1)octane-3,7-dione bis-(2-hydrazino-2-imidazoline)hydrazone dihydrobromide.

Figure XVII comprises a structure wherein R54 and R55 are independently selected from the group consisting of hydrogen, hydroxy (lower) alkyl, lower acyloxy (lower) alkyl, lower alkyl, or R54 and R55 together with their ring carbons may be an aromatic fused ring; Za is hydrogen or an amino group; Ya is hydrogen, or a group of the formula —CH2C (═O)— R56 wherein R is a lower alkyl, alkoxy, hydroxy, amino or aryl group; or a group of the formula —CHR′ wherein R′ is hydrogen, or a lower alkyl, lower alkynyl, or aryl group; and A is a halide, tosylate, methanesulfonate or mesitylenesulfonate ion.

The lower alkyl groups of the compounds of Formula XVII contain 1-6 carbon atoms and include methyl, ethyl, propyl, butyl, pentyl, hexyl, and the corresponding branched-chain isomers thereof. The lower alkynyl groups contain from 2 to 6 carbon atoms. Similarly, the lower alkoxy groups contain from 1 to 6 carbon atoms, and include methoxy, ethoxy, propoxy, butoxy, pentoxy, and hexoxy, and the corresponding branched-chain isomers thereof. These groups are optionally substituted by one or more halo, hydroxy, amino or lower alkylamino groups. 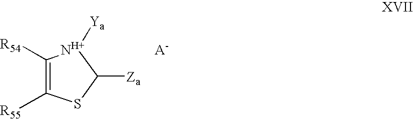
The lower acyloxy (lower) alkyl groups encompassed by the above Formula XVII include those wherein the acyloxy portion contain from 2 to 6 carbon atoms and the lower alkyl portion contains from 1 to 6 carbon atoms.

Typical acyloxy portions are those such as acetoxy or ethanoyloxy, propanoyloxy, butanoyloxy, pentanoyloxy, hexanoyloxy, and the corresponding branched chain isomers thereof. Typical lower alkyl portions are as described herein above. The aryl groups encompassed by the above formula are those containing 6-10 carbon atoms, such as phenyl and lower alkyl substituted-phenyl, e. g., tolyl and xylyl, and are optionally substituted by 1-2 halo, hydroxy, lower alkoxy or di (lower) alkylamino groups. Preferred aryl groups are phenyl, methoxyphenyl and 4-bromophenyl groups.

The halo atoms in the above Formula XVII may be fluoro, chloro, bromo, or iodo. For the purposes of this invention, the compounds of Formula XVII are formed as biologically and pharmaceutically acceptable salts. Useful salt forms are the halides, particularly the bromide and chloride, tosylate, methanesulfonate, and mesitylenesulfonate salts. Other related salts can be formed using similarly non-toxic, and biologically and pharmaceutically acceptable anions.

Of the compounds encompassed by Formula XVII, certain substituents are preferred. For instance, the compounds wherein R54 or R55 are lower alkyl groups are preferred. Also highly preferred are the compounds wherein Ya is a 2-phenyl-2-oxoethyl or a 2-[4′-bromophenyl]-2-oxoethyl group.

Representative of the compounds of Formula XVII are: 3-aminothiazolium mesitylenesulfonate; 3-amino-4,5-dimethylaminothiazolium mesitylenesulfonate; 2,3-diaminothiazolinium mesitylenesulfonate; 3-(2-methoxy-2-oxoethyl)-thiazolium bromide; 3-(2-methoxy-2-oxoethyl)-4,5-dimethylthiazolium bromide; 3-(2-methoxy-2-oxoethyl)-4-methylthiazolium bromide; 3-(2-phenyl-2-oxoethyl)-4-methylthizolium bromide; 3-(2-phenyl-2-oxoethyl)-4,5-dimethylthiazolium bromide; 3-amino4-methylthiazolium mesitylenesulfonate; 3-(2-methoxy-2-oxoethyl)-5-methylthiazolium bromide; 3-(3-(2-phenyl-2-oxoethyl)-5-methylthiazolium bromide; 3-[2-(4′-bromophenyl)-2-oxoethyl] thiazolium bromide; 3-[2-(4′-bromophenyl)-2-oxoethyl]-4-methylthiazolium bromide; 3-[2-(4′-bromophenyl)-2-oxoethyl]-5-methylthiazolium bromide; 3-[2-(4′bromophenyl)-2-oxoethyl]-4,5-dimethylthiazolium bromide; 3-(2-methoxy-2-oxoethyl)-4-methyl-5-(2-hydroxyethyl) thiazolium bromide; 3-(2-phenyl-2-oxoethyl)-4-methyl-5-(2-hydroxyethyl)thiazolium bromide; 3-[2-(4′-bromophenyl)-2-oxoethyl]-4-methyl-5-(2-hydroxyethyl)thiazolium bromide; 3,4-dimethyl-5-(2-hydroxyethyl)thiazolium iodide; 3-ethyl-5-(2-hydroxyethyl)-4-methylthiazolium bromide; 3-benzyl-5-(2-hydroxyethyl)-4-methylthiazolium chloride; 3-(2-methoxy-2-oxoethyl)benzothiazolium bromide; 3-(2-phenyl-2-oxoethyl)benzothiazolium bromide; 3-[2-(4′bromophenyl)-2-oxoethyl] benzothiazolium bromide; 3-(carboxymethyl)benzothiazolium bromide; 2,3-(diamino)benzothiazolium mesitylenesulfonate; 3-(2-amino-2-oxoethyl)thiazolium bromide; 3-(2-amino-2-oxoethyl)-4-methylthiazolium bromide; 3-(2-amino-2-oxoethyl)-5-methylthiazolium bromide; 3-(2-amino-2-oxoethyl)4,5-dimethylthiazolium bromide; 3-(2-amino-2-oxoethyl)benzothiazolium bromide; 3-(2-amino-2-oxoethyl)4-methyl-5-(2-hydroxyethyl)thiazolium bromide; 3-amino-5-(2-hydroxyethyl)-4-methylthiazolium mesitylenesulfonate; 3-(2-methyl-2-oxoethyl)thiazolium chloride; 3-amino4-methyl-5-(2-acetoxyethyl)thiazolium mesitylenesulfonate; 3-(2-phenyl-2-oxoethyl)thiazolium bromide; 3-(2-methoxy-2-oxoethyl)-4-methyl-5-(2-acetoxyethyl)thiazoliumbromide; 3-(2-amino-2-oxoethyl)-4-methyl-5-(2-acetoxyethyl)thiazolium bromide; 2-amino-3-(2-methoxy-2-oxoethyl)thiazolium bromide; 2-amino-3-(2-methoxy-2-oxoethyl) benzothiazolium bromide; 2-amino-3-(2-amino-2-oxoethyl)thiazolium bromide; 2-amino-3-(2-amino-2-oxoethyl)benzothiazolium bromide; 3-[2-(4′-methoxyphenyl)-2-oxoethyl]-thiazolinium bromide; 3-[2-(2′,4′-dimethoxyphenyl)-2-oxoethyl]-thiazolinium bromide; 3-[2-(4′-fluorophenyl)-2-oxoethyl]-thiazolinium bromide; 3-[2-(2′,4′-difluorophenyl)-2-oxoethyl]-thiazolinium bromide; 3-[2-(4′-diethylaminophenyl)-2-oxoethyl]-thiazolinium bromide; 3-propargyl-thiazolinium bromide; 3-propargyl-4-methylthiazolinium bromide; 3-propargyl-5-methylthiazolinium bromide; 3-propargyl-4,5-dimethylthiazolinium bromide; and 3-propargyl-4-methyl-5-(2-hydroxyethyl)-thiazolinium bromide.

Formula XVIII comprises a structure wherein, R57 is OH, NHCONCR61R62, or N═C(NR61R62)2; R61 and R62 are each independently selected from the group consisting of: hydrogen; C1-10 alkyl, straight or branched chain; aryl C1-4 alkyl; and mono- or di-substituted aryl C1-4 alkyl, where the substituents are fluoro, chloro, bromo, iodo or C1-10 alkyl, straight or branched chain; further wherein R58 and R59 are each independently selected from the group consisting of hydrogen, amino, and mono- or di-substituted amino where the substituents are C1-10 alkyl, straight or branched chain C3-8, cycloalkyl; provided that R58 and R59 may not both be amino or substituted amino; and R60 is hydrogen, trifluoromethyl; fluoro; chloro; bromo; or iodo; or a pharmaceutically acceptable salt thereof.

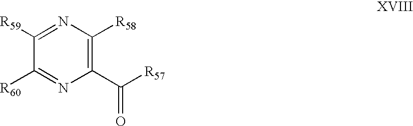

In another aspect of the invention, the inhibitor of 3DG function can be a compound such as the amino acid arginine, which reacts irreversibly with 3DG to form a five membered ring called an imidazolone. Once the reaction occurs, 3DG cannot cause crosslinking because the active crosslinker has been removed. Thus, the binding of arginine with 3DG prevents protein crosslinking (see Example 18 and FIG. 12). As described herein, treatment of collagen with 3DG causes the collagen to migrate electrophoretically as if it had a higher molecular weight, which is indicative of crosslinking. However, treatment of a sample of collagen with 3DG in the presence of arginine prevented the appearance of more slowly migrating proteins (Example 18 and FIG. 12). Arginine should be construed to inhibit other alpha-dicarbonyl sugars as well. The invention should be construed to include not just arginine, but it should also be construed to include derivatives and modifications thereof. In one aspect of the invention, arginine may be derivatized or modified to ensure greater efficiency of penetration or passage into the skin or other tissues or to ensure a more efficacious result.

The amino acid arginine has the structure:

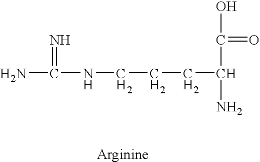

In yet another aspect of the invention, the inhibitor of 3DG or other alpha-dicarbonyl sugar function may be L-cysteine or a derivative such as an α-amino-β,β-mercapto-β,β-dimethyl-ethane, or a derivative or modification thereof. Members of the α-amino-β,β-mercapto-β,β-dimethyl-ethane family include, but are not limited to, compounds such as D-penicillamine, L-penicillamine, and D,L-penicillamine (see Jacobson et al., WO 01/78718). The functions inhibited include, but are not limited to, the various functions described herein, such as inhibiting crosslinking of proteins and other molecules, as well as other functions which cause damage to molecules such as proteins, lipid and DNA. For example, damage to lipids may include lipid peroxidation and damage to DNA may include damage such as mutagenesis.

In one aspect of the invention, an α-amino-β,β-mercapto-β,β-dimethyl-ethane may be derivatized or modified to ensure greater efficiency of penetration or passage into the skin or other tissues or to ensure greater efficiency in inhibiting the desired function of 3DG and other alpha-dicarbonyl sugars.

For example, the α-amino-β,β-mercapto-β,-dimethyl-ethane derivative, D-penicillamine, has the structure: 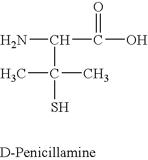
It should be understood that the compounds described herein are not the only compounds capable of inhibiting desmosine productin. It will be recognized by one of skill in the art that the various embodiments of the invention as described herein related to inhibition of desmosine function, also encompass other methods and compounds useful for inhibiting desmosine function. It will also be recognized by one of skill in the art that other compounds and techniques can be used to practice the invention.

In another embodiment of the invention, as discussed elsewhere herein, any of the compounds or methods set forth or taught herein are used to prevent or treat skin aging, wrinkling and loss of elasticity.

In one aspect of the invention, various changes in the skin can be measured following treatment with compounds that inhibit the production of desmosines, by inhibiting fructosamine 3 kinase and 3DG. The skin topography can be defined by parameters such as: (a) number of wrinkles; (b) total area of wrinkles; (c) total length of wrinkles; (d) mean length of wrinkles; and (e) mean depth of wrinkles. The type of wrinkles can be determined on the basis of depth, length, and area. These properties can be used when evaluating the changes in skin due to disease or disorder or the effects of a treatment on the skin. The effects of changes in elastin and function on various skin qualities can be determined based on techniques known in the art. Methods to measure skin quality include, but are not limited to, measuring viscoelastic properties with instruments such as a ballistometer, measuring the mechanical/vertical deformation properties of the skin with an instrument such as a cutometer, or measuring changes in skin capacitance resulting from changes in the degree of hydration using a corneometer.

The present invention also relates to the reversal of protein crosslinking in a mammal. In one embodiment, the invention relates to reversal or cleavage of cross-links formed within a single protein or between two or more proteins as a consequence of the formation of advanced glycosylation (glycation) end products. In one aspect, the present invention features compounds and methods useful in the reversal of collagen and elastin. In another embodiment, the present invention features compositions and methods useful in the reversal of protein crosslinking resulting from diabetic complications, as such complications are described in greater detail elsewhere herein.

Therefore, one embodiment of the present invention features a method of treating a mammal having a disease selected from the group consisting of scleroderma, keloids, and scarring, wherein the mammal is in need of such a treatment. The method comprises administering to the mammal an effective amount of a composition comprising at least one compound capable of disrupting a crosslinkage between crosslinked proteins. Examples of compounds and methods useful in the present invention can be found in, for example, U.S. Pat. No. 6,319,934, which is incorporated herein by reference. When armed with the disclosure set forth in the present application for the first time, the skilled artisan will know how to apply compounds and methods of U.S. Pat. No. 6,319,934 to the present invention.

In one aspect of the invention, a compound useful in the method is selected from the group consisting of compounds of the formula XXV: 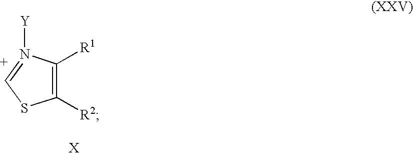
wherein R.sup.1 and R.sup.2 are independently selected from the group consisting of hydrogen and an alkyl group, which can be substituted by a hydroxy group; Y is a group of the formula —CH.sub.2 C(═O)R wherein R is a heterocyclic group other than alkylenedioxyaryl containing 4-10 ring members and 1-3 heteroatoms selected from the group consisting of oxygen, nitrogen and sulfur, the heterocyclic group can be substituted by one or more substituents selected from the group consisting of alkyl, oxo, alkoxycarbonylalkyl, aryl, and aralkyl groups; and said one or more substituents can be substituted by one or more alkyl or alkoxy groups; or group of the formula —CH.sub.2 C(.dbd.O)—NHR′ wherein R′ is a heterocyclic group other than alk-ylenedioxyaryl containing 4-10 ring members and 1-3 heteroatoms selected from the group consisting of oxygen, nitrogen, and sulfur, the heterocyclic group can be substituted by one or more alkoxycarbonylalkyl groups; and X is a pharmaceutically acceptable ion; and a carrier therefor.

The invention relates to the administration of an identified compound in a pharmaceutical or cosmetic composition to practice the methods of the invention, the composition comprising the compound or an appropriate derivative or fragment of the compound and a pharmaceutically acceptable carrier. The invention should be construed to include the use of one, or simultaneous use of more than one, lysine generation. When more than one stimulator or inhibitor is used, they can be administered together or they can be administered separately.

In one embodiment, the pharmaceutical compositions useful for practicing the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 100 g/kg/day.

Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparin sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer compounds according to the methods of the invention.

Compounds which are identified using any of the methods described herein may be formulated and administered to a mammal for treatment of skin aging, s skin wrinkling, and loss of skin elasticity.

Such a pharmaceutical composition may consist of the active ingredient alone, in a form suitable for administration to a subject, or the pharmaceutical composition may comprise at least one active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology. Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see Constanza, U.S. Pat. No. 6,323,219). The source of active compound to be formulated will generally depend upon the particular form of the compound. Small organic molecules and peptidyl or oligo fragments can be chemically synthesized and provided in a pure form suitable for pharmaceutical/cosmetic usage. Products of natural extracts can be purified according to techniques known in the art. Recombinant sources of compounds are also available to those of ordinary skill in the art.

In alternative embodiments, the topically active pharmaceutical or cosmetic composition may be optionally combined with other ingredients such as moisturizers, cosmetic adjuvants, anti-oxidants, chelating agents, bleaching agents, tyrosinase inhibitors and other known depigmentation agents, surfactants, foaming agents, conditioners, humectants, wetting agents, emulsifying agents, fragrances, viscosifiers, buffering agents, preservatives, sunscreens and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art. The compositions of this invention may also contain active amounts of retinoids (i.e., compounds that bind to any members of the family of retinoid receptors), including, for example, tretinoin, retinol, esters of tretinoin and/or retinol and the like.

The topically active pharmaceutical or cosmetic composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically-or naturally derived.

Liquid derivatives and natural extracts made directly from biological sources may be employed in the compositions of this invention in a concentration (w/v) from about 1 to about 99%. Fractions of natural extracts and protease inhibitors may have a different preferred rage, from about 0.01% to about 20% and, more preferably, from about 1% to about 10% of the composition. Of course, mixtures of the active agents of this invention may be combined and used together in the same formulation, or in serial applications of different formulations.

The composition of the invention may comprise a preservative from about 0.005% to 2.0% by total weight of the composition. The preservative is used to prevent spoilage in the case of an aqueous gel because of repeated patient use when it is exposed to contaminants in the environment from, for example, exposure to air or the patient's skin, including contact with the fingers used for applying a composition of the invention such as a therapeutic gel or cream. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound for use in the invention in the aqueous gel formulation. Preferred antioxidants for some compounds are BHT, BHA, alphatocopherol and ascorbic acid in the preferred range of about 0.01% to 0.3% and more preferably BHT in the range of 0.03% to 0. 1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Controlled-release preparations may also be used and the methods for the use of such preparations are known to those of skill in the art.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, that are adapted for controlled-release are encompassed by the present invention.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin.

Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but S are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

As used herein, an “oily” liquid is one which comprises a carbon-containing liquid molecule and which exhibits a less polar character than water.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The terms oral rinse and mouthwash are used interchangeably herein.

A pharmaceutical composition of the invention may be prepared, packaged, or sold in a formulation suitable for oral or buccal administration. Such a formulation may comprise, but is not limited to, a gel, a liquid, a suspension, a paste, toothpaste, a mouthwash or oral rinse, and a coating. For example, an oral rinse of the invention may comprise a compound of the invention at about 1.4%, chlorhexidine gluconate (0.12%), ethanol (11.2%), sodium saccharin (0.15%), FD&C Blue No. 1 (0.001%), peppermint oil (0.5%), glycerine (10.0%), Tween 60 (0.3%), and water to 100%. In another embodiment, a toothpaste of the invention may comprise a compound of the invention at about 5.5%, sorbitol, 70% in water (25.0%), sodium saccharin (0.15%), sodium lauryl sulfate (1.75%), carbopol 934, 6% dispersion in (15%), oil of spearmint (1.0%), sodium hydroxide, 50% in water (0.76%), dibasic calcium phosphate dihydrate (45%), and water to 100%. The examples of formulations described herein are not exhaustive and it is understood that the invention includes additional modifications of these and other formulations not described herein, but which are known to those of skill in the art.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets S include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject.

Douche preparations may further comprise various additional ingredients including, but not limited to, antioxidants, antibiotics, antifungal agents, and preservatives. As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a is tissue-penetrating non-surgical wound, and the like. In particular, parenteral administration is contemplated to include, but is not limited to, subcutaneous, intraperitoneal, intramuscular, intrastemal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are usefull include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or Vetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

It will be recognized by one of skill in the art that the various embodiments of the invention as described above relating to methods of inhibiting 3DG or treating 3DG related diseases or conditions, includes other diseases and conditions not described herein.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

Isolation and Identification of FL3P:

The following assays were performed in order to verify that fructoselysine (FL) could be identified in its phosphorylated state, e.g., FL3P. A 31P NMR analysis of a perchloric acid extract of diabetic rat kidneys was performed and showed a new sugar monophosphate resonance at 6.24 ppm which is not observed in non-kidney tissue and is present at greatly reduced levels in non-diabetic kidney. The compound responsible for the observed resonance was isolated by chromatography of the extract on a microcrystalline cellulose column using 1-butanol-acetic acid-water (5:2:3) as eluent. The structure was determined by proton 2D COSY to be fructoselysine 3-phosphate. This was later confirmed by injecting animals with FL, prepared as previously described (Finot and Mauson, 1969, Helv. Chim. Acta, 52:1488), and showing direct phosphorylation to FL3P.

The use of FL specifically deuterated in position-3 confirmed the position of the phosphate at carbon-3. This was performed by analyzing the 31P NMR spectra, both coupled and decoupled. The normal P—O—C—H coupling produces a doublet in FL3P with a J value of 10.3 Hz; whereas P—O—C—D has no coupling and produces a singlet both coupled and decoupled, as was found for 3-deuterated FL3P. A unique property of FL3P is that when treated with sodium borohydride it is converted into two new resonances at 5.85 and 5.95 ppm, which correspond to mannitol and sorbitol-lysine 3-phosphates.

### Example 2

Synthesis of FL3P:

1 mmol of dibenzyl-glucose 3-phosphate and 0.25 mmol of α-carbobenzoxy-lysine was refluxed in 50 ml of MeOH for 3 hours. The solution was diluted with 100 ml water and chromatographed on a Dow-50 column (2.5×20 cm) in the pyridinium form and eluted first with water (200 ml) and then with 600 ml buffer (0.1M pyridine and 0.3M acetic acid). The target compound eluted-at the end of the water wash and the beginning of the buffer wash. The results demonstrated that removal of the cbz and benzyl blocking groups with 5% Pd/C at 20 psi of hydrogen gave FL3P in 6% yield.

### Example 3

Enzymatic Production of FL3P from FL and ATP and Assay for Screening Inhibitors.

Initially, ³¹P NMR was used to demonstrate kinase activity in the kidney cortex. A 3 g sample of fresh pig kidney cortex was homogenized in 9 ml of 50 mM Tris.HCl containing 150 mM KCl, 5 mM DTT, 15 mM MgCl2, pH 7.5. This was centrifuged at 10,000 g for 30 minutes, and then the supernatant was centrifuged at 100,000 g for 60 minutes. Ammonium sulfate was added to 60% saturation. After 1 hour at 4° C. the precipitate was collected by centrifugation and dissolved in 5 ml of original buffer. A 2 ml aliquot of this solution was incubated with 10 mM ATP and 10 mM of FL (prepared as in Example 1, above) for 2 hours at 37° C. The reaction was quenched with 300 μl of perchloric acid, centrifuged to remove protein, and desalted on a column of Sephadex G 10(5×10 cm). 31P NMR analysis of the reaction mixture detected formation of FL3P.

Based on the proof of kinase activity thus obtained, a radioactive assay was developed. This assay was designed to take advantage of the binding to Dow-50 cation exchange resin by FL3P. This characteristic of FL3P was discovered during efforts to isolate it. Since most phosphates do not bind to this resin, it was suspected that the bulk of all compounds that react with ATP as well as any excess ATP would not be bound. The first step was to determine the amount of resin required to remove the ATP in the assay. This was accomplished by pipetting the mixture into a suspension of 200 mg of Dow-1 in 0.9 ml H2O, vortexing, and centrifuging to pack the resin. From this 0.8 ml of supernatant was pipetted onto 200 mg of fresh dry resin, vortexed and centrifuged. A 0.5 ml volume of supernatant was pipetted into 10 ml of Ecoscint A and counted. Residual counts were 85 cpm. This procedure was used for the assay. The precipitate from 60% ammonium sulfate precipitation of the crude cortex homogenate was redissolved in the homogenate buffer at 4° C. The assay contains 10 mM γ33P-ATP (40,000 cpm), 10 mM FL, 150 mM KCl, 15 mM MgCl2, 5 mM DTT in 0.1 ml of 50 mM Tris.HCl, pH 7.5. The relationship between rates of FL3P production and enzyme concentration was determined using triplicate determinations with 1, 2, and 4 mg of protein for 30 minutes at 37° C. Blanks run concurrently without FL were subtracted and the data recorded. The observed activity corresponds to an approximate FL3P synthesis rate of 20 nmols/hr/mg protein.

### Example 4

Inhibition of the Formation of Free Lysine as Measured by Meglumine, the Formation of 3DG and Various Polyollysines.

a. General Polyollysine Synthesis:

The sugar (11 mmoles), α-carbobenzoxy-lysine (10 mmols) and NaBH3CN (15 mmoles) were dissolved in 50 ml of MeOH—H2O (3:2) and stirred at 25° C. for 18 hours. The solution was treated with an excess of Dow-50 (H) ion exchange resin to decompose excess NaBH3CN. This mixture (liquid plus resin) was transferred onto a Dow-50 (H) column (2.5×15 cm) and washed well with water to remove excess sugar and boric acid. The carbobenzoxy-polyollysine was eluted with 5% NH4OH. The residue obtained upon evaporation was dissolved in water-methanol (9:1) and reduced with hydrogen gas (20 psi) using a 10% palladium on charcoal catalyst. Filtration and evaporation yields the polyollysine.

b. Experimental Protocol for Reduction of Urinary and Plasma 3-Deoxyglucosone by Sorbitollysine, Mannitollysine and galactitollysine:

Urine was collected from six rats for three hours. A plasma sample was also obtained. The animals were then given 10 μmols of either sorbitollysine, mannitollysine, or galactitollysine by intraperitoneal injection. Urine was collected for another three hours, and a plasma sample obtained at the end of the three hours. 3-deoxyglucosone was measured in the samples, as described in Example 5, below, and variable volumes were normalized to creatinine. The average reduction of urinary 3-deoxyglucosone was 50% by sorbitollysine, 35% by mannitollysine and 35% by galactitollysine. Plasma 3-deoxyglucosone was reduced 40% by sorbitollysine, 58% by mannitollysine and 50% by galactitollysine.

c Use of Meglumine to Reduce Urinary 3-Deoxyglucosone:

Three rats were treated as in b), immediately above, except meglumine (100 μmols) was injected intraperitoneally instead of the above-mentioned lysine derivatives. Three hours after the injection the average 3-deoxyglucosone concentrations in the urine were decreased 42%.

### Example 5

Elevation of Urinary FL, 3DG and 3DF in Humans Following Ingestion of Glycated Protein.

a. Preparation of Glycated Protein Containing Food Product:

260 g of casein, 120 g of glucose and 720 ml of water were mixed to give a homogeneous mixture. This mixture was transferred to a metal plate and heated at 65° C. for 68 hours. The resulting cake was then pulverized to a coarse powder. This powder contained 60% protein as determined by the Kjeldahl procedure.

b. Measurement of Glycated Lysine Content:

One gram of the powder prepared as in step a., above, was hydrolyzed by refluxing with 6N HCl for 20 hours. The resulting solution was adjusted to pH 1.8 with NaOH solution and diluted to 100 ml. The fructoselysine content was measured on an amino acid analyzer as furosine, the product obtained from acid hydrolysis of fructoselysine. In this way, it was determined that the cake contained 5.5% (w/w) fructoselysine.

c. Experimental Protocol:

Volunteers spent two days on a fructoselysine-free diet and then consumed 22.5 g of the food product prepared as described herein, thus effectively receiving a 2 gram dose of fructoselysine. Urine-was collected at 2 hour intervals for 14 hours and a final collection was made at 24 hours.

d. Measurement of FL, 3DG and 3DF in Urine:

FL was measured by HPLC with a Waters 996 diode Array using a Waters C18 Free Amino Acid column at 460° C. and a gradient elution system of acetonitrile-methyl alcohol-water (45:15:40) into acetonitrile-sodium acetate-water (6:2:92) at 1 ml/min. Quantitation employed an internal standard of meglumine. 3DF was measured by HPLC after deionization of the sample. Analyses were performed on a Dionex DX-500 HPLC system employing a PA1 column (Dionex) and eluting with 32 mM sodium hydroxide at 1 ml/min. Quantitation was performed from standard curves obtained daily with synthetic 3DF.

3DG was measured by GC-MS after deionization of the sample. 3DG was derivatized with a 10-fold excess of diaminonaphthalene in PBS. Ethyl acetate extraction gave a salt free fraction which was converted to the trimethyl silyl ethers with Tri-Sil (Pierce). Analysis was performed on a Hewlett-Packard 5890 selected ion monitoring GC-MS system. GC was performed on a fused silica capillary column (DB-5,25 mx.25 mm) using the following temperature program: injector port 250° C., initial column temperature 150° C. which was held for 1 minute, then increased to 290° C. at 16° C./minute and held for 15 minutes. Quantitation of 3DG employed selected ion monitoring using an internal standard of U-13C-3DG.

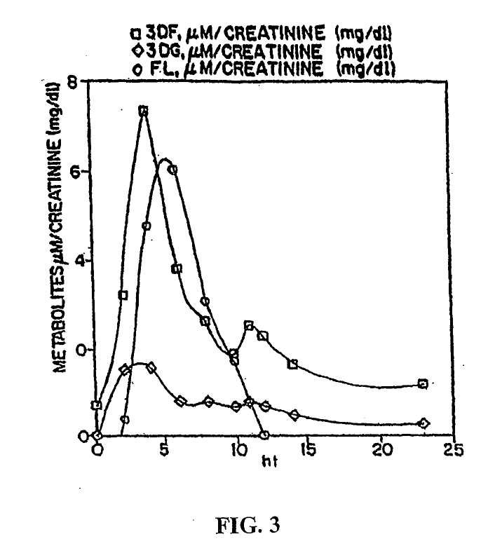

The graph depicted in FIG. 3 represents production of FL, 3DF, and 3DG in the urine of one volunteer after consuming the glycated protein. The rapid appearance of all three metabolites is clearly evident. Both 3DF and 3DG show a slight elevation even after twenty-four hours.

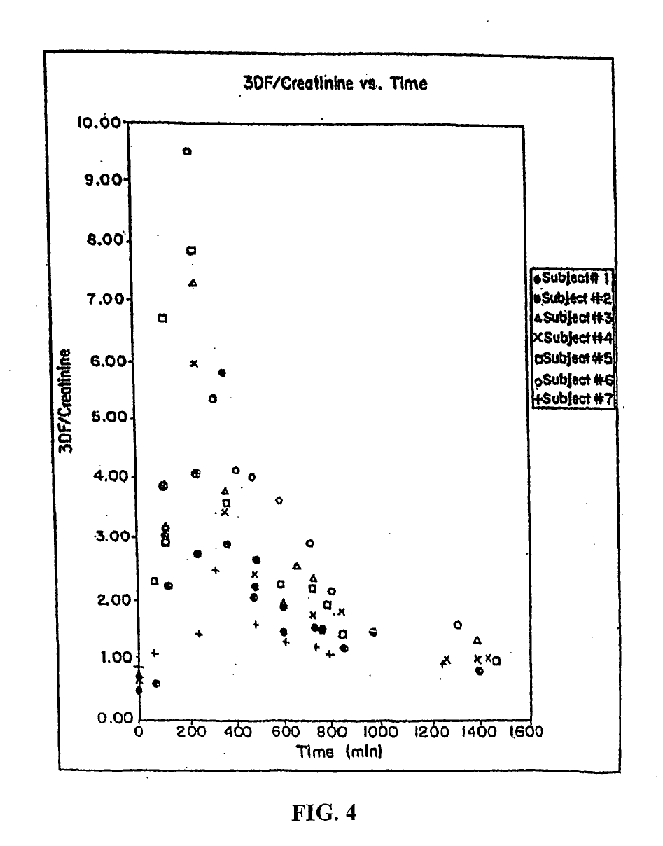

The graph shown in FIG. 4 represents the formation of 3DF in each of the members of a seven-person test group. A similar pattern was seen in all cases. As demonstrated in FIG. 4, 3DF excretion peaks about 4 hours after the FL bolus and a slight elevation of 3DF is noticeable even 24 h after the bolus.

### Example 6

Effects of Increased Dietary Uptake of Glycated Proteins.

N-acetyl-β-glucosaminidase (NAGase) is an enzyme excreted into the urine in elevated concentration in diabetics. It is thought to be an early marker of tubular damage, but the pathogenesis of increased NAGase in urine is not well understood. The increased urinary output of NAGase in diabetics has been proposed to be due to activation of lysosomes in proximal tubules induced by diabetes with an increased output into the urine rather than destruction of cells.

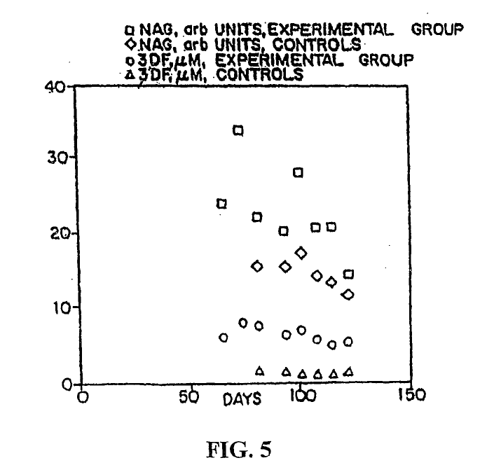

Rats were fed a diet containing 0.3% glycated protein or control feed over several months. The urinary output of NAGase and 3DF were determined at various times, as indicated in FIG. 5. The amount of 3DG excreted in urine was also determined.

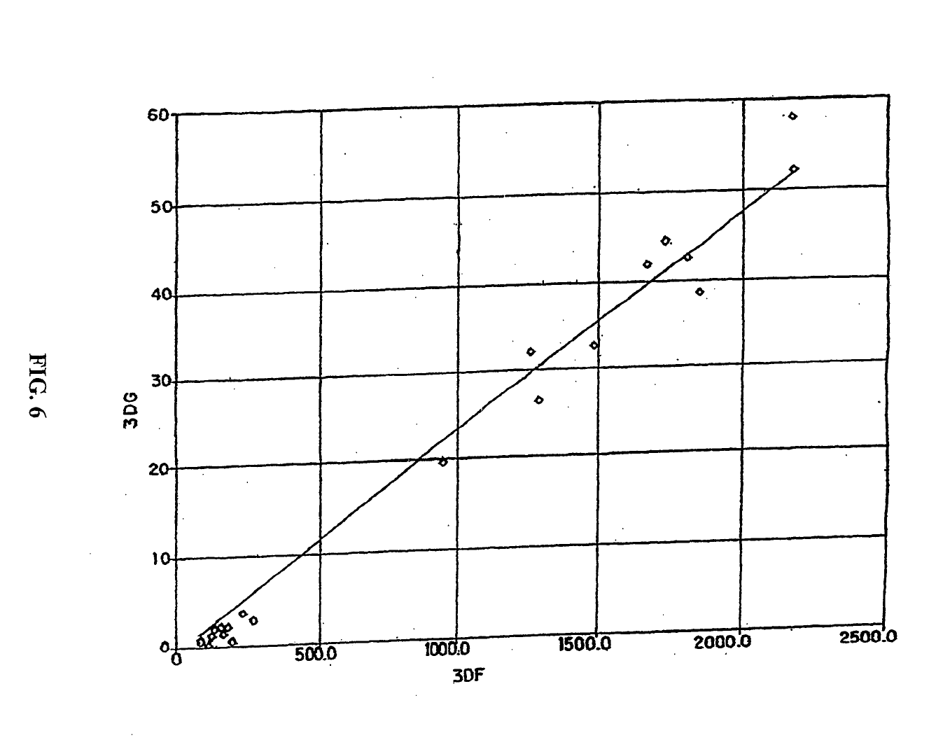

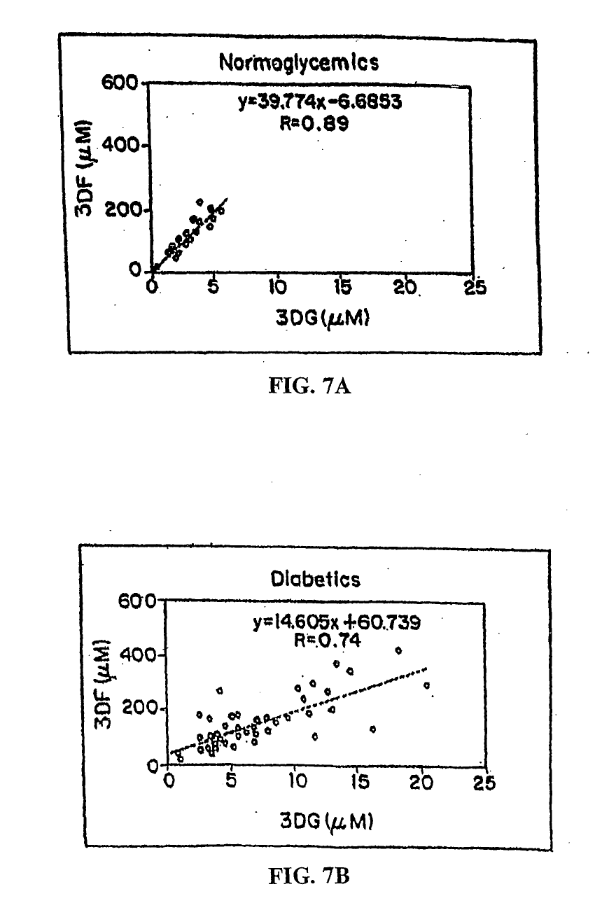

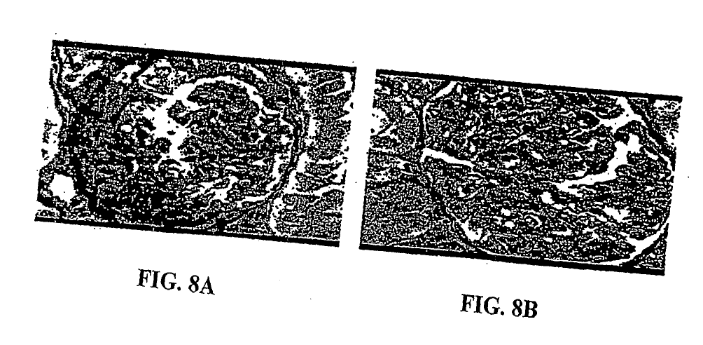

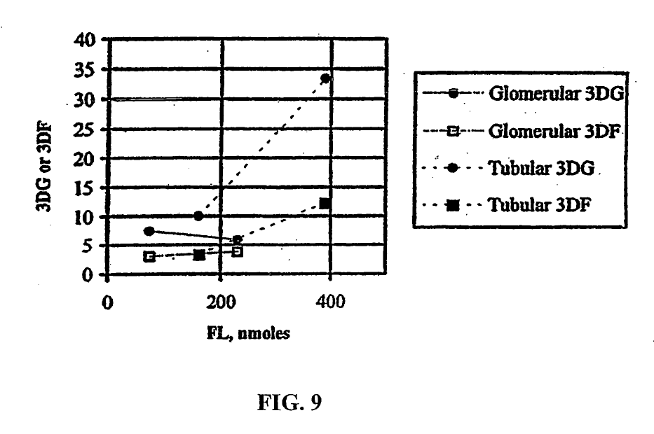

The results obtained in this example demonstrate that in all comparisons 3DF and NAGase levels are elevated in the experimental group relative to the control. Thus, animals fed glycated protein excrete excess NAGase into their urine, similar to results obtained with diabetics. NAGase output increased by approximately 50% in the experimental group, compared with control animals. The experimental animals also had a five-fold increase in urine 3DF compared with controls. Urinary 3DF was found to correlate extremely well with 3DG, as can be seen in FIGS. 5 and 6.

### Example 7

Electrophoretic Analysis of Kidney Proteins.

Two rats were injected daily with 5 μmols of either FL or mannitol (used as a control) for 5 days. The animals were sacrificed and the kidneys removed and dissected into the cortex and medulla. Tissues were homogenized in 5 volumes of 50 mM Tris.HCl containing 150 mM KCl, 15 mM MgCl2 and 5 mM DTT, pH 7.5. Cellular debris was removed by centrifugation at 10,000×g for 15 minutes, and the supernatant was then centrifuged at 150,000×g for 70 minutes. The soluble proteins were analyzed by SDS PAGE on 12% polyacrylamide gels as well as on 4-15 and 10-20% gradient gels.

It was found that in all cases, lower molecular weight bands were missing or visually reduced from the kidney extract of the animal injected with FL when compared with the animal injected with mannitol.

### Example 8

Synthesis of 3-O-methylsorbitollysine (Structure XIX).

3-OMe glucose (25 grams, 129 mmol) and α-Cbz-lysine (12 grams, 43 mmol) were dissolved in 200 ml of water-methanol (2:1). Sodium cyanoborohydride (10 grams, 162 mmol) was added and the reaction stirred for 18 days at room temperature. Reaction of α-Cbz-lysine was monitored by thin layer chromatography on silica gel employing 1-butanol-acetic acid-water (4:1:1) using ninhydrin for visualization. The reaction was complete when no α-Cbz-lysine remained. The solution was adjusted to pH 2 with HCl to decompose excess cyanoborohydride, neutralized and then applied to a column (5×50 cm) of Dowex-50 (H+) and the column washed well with water to remove excess 3-O-me-glucose. The target compound was eluted with 5% ammonium hydroxide. After evaporation the residue was dissolved in 50 ml of water-methanol (2:1) and 10% Pd/C (0.5 gram) was added. The mixture was shaken under 20 psi of hydrogen for 1 hr. The charcoal was filtered off and the filtrate evaporated to a white powder (10.7 gram, 77% yield based on α-Cbz-lysine) that was homogeneous when analyzed by reversed phase HPLC as the phenylisothiocyanate derivative. Elemental analysis: Calculated for C13H28N2O7.CH3OH.2H2OC, 42.86; H, 9.18; N, 7.14. Found: C, 42.94; H, 8.50; N, 6.95.

Other specific compounds having the structure of formula (XIX), above, may be made, e.g., by glycation of a selected nitrogen- or oxygen-containing starting material, which may be an amino acid, polyaminoacid, peptide or the like, with a glycating agent, such as fructose, which may be chemically modified, if desired, according to procedures well know to those skilled in the art.

### Example 9

Additional Assay for FL3P Kinase Activity.

a. Preparation of Stock Solutions:

An assay buffer solution was prepared which was 100 mM HEPES pH 8.0, 10 mM ATP, 2 mM MgCl2, 5 mM DTT, 0.5 mM PMSF. A fructosyl-spermine stock solution was prepared which was 2 mM fructosyl-spermine HCl. A spermine control solution was prepared which was 2 mM spermine HCl.

b. Synthesis of Fructosyl-spermine:

Synthesis of fructosyl-spermine was performed by an adaptation of a known procedure (J. Hodge and B. Fisher, 1963, Methods Carbohydr. Chem., 2:99-107). A mixture of spermine (500 mg), glucose (500 mg), and sodium pyrosulfite (80 mg) was prepared in a molar ratio of 8:4:1 (spermine: glucose: pyrosulfite) in 50 ml of methanol-water (1:1) and refluxed for 12 hours. The product was diluted to 200 ml with water and loaded onto a DOW-50 column (5×90 cm). The unreacted glucose was removed by 2 column volumes of water and the product and unreacted spermine were removed with 0.1 M NH4OH. Pooled peak fractions of the product were lyophilized and concentration of fructosyl-spermine was determined by measuring the integral of the C-2 fructosyl peak in a quantitative ¹³C NMR spectrum of the product (NMR data collected with a 45° pulse, a 10 second relaxation delay and without NOE decoupling).

c. Kinase Assay to Determine Purification:

An incubation mixture was prepared including 10 μl of the enzyme preparation, 10 μl of assay buffer, 1.0 uCi of 33P ATP, 10 μl of fructosyl-spermine stock solution and 70 μl of water and incubated at 37° C. for 1 hour. At the end of the incubation 90 μl (2×45 μl) of the sample was spotted onto two 2.5 cm diameter cellulose phosphate disks (Whatman P-81) and allowed to dry. The disks were washed extensively with water. After drying, the disks were placed in scintillation vials and counted.

Each enzyme fraction was assayed in duplicate with an appropriate spermine control.

### Example 10

Kidney Pathology Observed in Test Animals on Glycated Protein Diet.

Three rats were maintained on a glycated protein diet (20% total protein; 3% glycated) for 8 months and compared to 9 rats of the same age maintained on a control diet. The glycated protein diet consisted of a standard nutritious diet to which 3% glycated protein had been substituted for nonglycated protein. The glycated protein was made by mixing together casein and glucose (2:1), adding water (2× the weight of the dried material), and baking the mixture at 60° C. for 72 hours. The control was prepared in the same way except that no water was used and the casein and glucose were not mixed prior to baking.

The primary finding was a substantial increase in damaged glomeruli in the animals on the glycated diet. Typical lesions observed in these animals were segmental sclerosis of the glomerular tuft with adhesion to Bowman's capsule, tubular metaplasia of the parietal epithelium and interstitial fibrosis. All animals on the glycated protein diet, and only one of the animals on the control diet showed more than 13% damaged glomeruli. The probability of this happening by chance is less than 2%. In addition to the pathological changes observed in the glomeruli, a number of hyalinated casts within tubules were observed. More of these hyalinated casts were found in animals on the glycated diet, although these were not quantitated. Increased levels of NAGase were also observed in the animals on the glycated diet.

Based on the results of this experiment, the glycated diet appeared to cause the test animals to develop a series of histological lesions similar to those seen in the diabetic kidney.

### Example 11

Urinary Excretion of 3-Deoxy-Fructose is Indicative of Progression to Microalbuminuria in Patients with Type I Diabetes.

As set forth herein, serum levels of the glycation intermediate, three deoxy-glucosone (3DG) and its reductive detoxification product, three deoxy-fructose (3DF), are elevated in diabetes. The relationship between baseline levels of these compounds and subsequent progression of microalbuminuria (MA) has been examined in a group of 39 individuals from a prospective cohort of patients at the Joslin Diabetes Center with insulin-dependent diabetes mellitus (IDDM) and microalbuminuria (based on multiple measurements during the two years of baseline starting between 1990-1993) and not on ACE inhibitors.

Baseline levels of 3DF and 3DG in random spot urines were measured by HPLC and GC-MS. Individuals that progressed to either a higher level of MA or proteinuria in the next four years (n=24) had significantly higher baseline levels of log 3DF/urinary creatinine ratios compared to non-progressors (n=15) (p=0.02). Baseline levels determined in this study were approximately 0.24 μmole/mg of creatinine in the progressors vs. approximately 0.18 μmole/mg of creatinine ratios in the non-progressors. Baseline 3DG/urine creatinine ratios did not differ between the groups. Adjustment of the baseline level of HgAIc (the major fraction of glycosylated hemoglobin) did not substantially alter these findings. These results provide additional evidence of the association between urinary 3DF and progression of kidney complications on diabetes.

a. Quantification of 3-deoxyfructose:

Samples were processed by passing a 0.3 ml aliquot of the test sample through an ion-exchange column containing 0.15 ml of AG 1-X8 and 0.15 ml of AG 50W-X8 resins. The columns were then washed twice with 0.3 ml deionized water, aspirated to remove free liquid and filtered through a 0.45 mm Millipore filter. Injections (50 μl) of the treated samples were analyzed using a Dionex DX 500 chromatography system. A carbopac. PA1 anion-exchange column was employed with an eluant consisting of 16% sodium hydroxide (200 mM) and 84% deionized water. 3DF was detected electrochemically using a pulsed amperometric detector. Standard 3DF solutions spanning the anticipated 3DF concentrations were run both before and after each unknown sample.

b. Measurement of Urine Creatinine:

Urine creatinine concentrations were determined by the end-point colorimetric method (Sigma Diagnostic kit 555-A) modified for use with a plate reader. Creatinine concentrations were assessed to normalize urine volumes for measuring metabolite levels present therein.

c. Measurement of Albumin in the Urine:

To assess albumin levels in the urine of the test subjects, spot urines were collected and immunonephelometry performed on a BN 100 apparatus with the N-albumin kit (Behring). Anti-albumin antibodies are commercially available. Albumin levels in urine may be assessed by any suitable assay including but not limited to ELISA assays, radioimmunoassays, Western, and dot blotting.

Based on the data obtained in the study of the Joslin Diabetes Center patients, it appears that elevated levels of urinary 3DF are associated with progression to microalbuminuria in diabetes. This observation provides a new diagnostic parameter for assessing the likelihood of progression to serious kidney complications in patients afflicted with diabetes.

### Example 12

3-O-methyl Sorbitollysine Lowers Systemic Levels of 3DG in Normal and Diabetic Rats.

A cohort of twelve diabetic rats was divided into two groups of six. The first group received saline-only injections, and the second received injections of 3-O-methyl sorbitollysine (50 mg/kg body weight) in saline solution. The same procedure was conducted on a cohort of twelve non-diabetic rats.

As summarized in Table B, within one week, the 3-O-methyl sorbitollysine treatment significantly reduced plasma 3DG levels as compared to the respective saline controls in both diabetic and non-diabetic rats.

TABLE B
3-O-Methyl sorbitollysine (3-OMeSL) reduces plasma 3DG levels in
diabetic and non-diabetic rats.

| | Diabetic rats | Non-diabetic rats |
|---|---|---|
| Saline only | 0.94 ± 0.28 uM | 0.23 ± 0.07 uM |
| | (n = 6) | (n = 6) |
| 3-Ome | 0.44 ± 0.10 uM | 0.13 ± 0.02 uM |
| | (n = 6) | (n = 7) |
| % Reduction | 53% | 43% |
| t-test | p = 0.0006 | p = 0.0024 |

The ability of 3-O-methyl sorbitollysine to reduce systemic 3DG levels indicates that diabetic complications other than nephropathy (e.g., retinopathy and stiffening of the aorta) may also be controllable by Amadorase inhibitor therapy.

### Example 13

Locus of 3-O-methyl Sorbitollysine Uptake in Vivo is the Kidney.

Six rats were injected intraperitoneally with 13.5 nmoles (4.4 mg) of 3-O-methyl sorbitollysine. Urine was collected for 3 hours, after which the rats were sacrificed. The tissues to be analyzed were removed and freeze clamped in liquid nitrogen. Perchloric acid extracts of the tissues were used for metabolite analysis. The tissues examined were taken from the brain, heart, muscle, sciatic nerve, spleen, pancreas, liver, and kidney. Plasma was also analyzed.

The only tissue extract found to contain 3-O-methyl sorbitollysine was that of the kidney. The urine also contained 3-O-methyl sorbitollysine, but plasma did not. The percentage of the injected dose recovered from urine and kidney varied between 39 and 96%, as shown in Table C, below.

TABLE C

| | nmols | nmols | nmols | total | % |
|---|---|---|---|---|---|
| | 3OMeSL* | 3OmeSL | 3OMeSL | 3OMeSL | 3OMeSL |
| Rat # | Injected | in urine | in kidneys | recovered | recovered |
| 2084 | 13500 | 2940 | 10071 | 13011 | 96.4 |
| 2085 | 13500 | 1675 | 6582 | 8257 | 61.2 |
| 2086 | 13500 | 1778 | 5373 | 7151 | 53.0 |
| 2087 | 13500 | 2360 | 4833 | 7193 | 53.3 |
| 2088 | 13500 | 4200 | 8155 | 12355 | 91.5 |
| 2089 | 13500 | 1355 | 3880 | 5235 | 38.8 |

*3-O-methyl sorbitollysine

### Example 14

Amadorase/fructosamine Kinase Activity Accounts for a Majority of 3DG Production.

Enzymatic production of 3DG was demonstrated in an in vitro assay with various key components (10 mM Mg-ATP, partially purified Amadorase, 2.6 mM FL) omitted from the reaction in order to assess their importance in 3DG production.

The results show that 3DG production is 20-fold higher in the presence of kidney extract containing Amadorase and its substrates (compare Table D, reactions 1 and 3). Clearly, the vast majority of 3DG production is enzymatically mediated in the presence of Amadorase.

TABLE D
Amadorase-dependent production of 3DG after 24 hours

| Reaction | Amadorase | ATP | FL (mM) | FL3P (mM) | 3DG (mM) |
|---|---|---|---|---|---|
| 1 | + | + | 2.6 | 0.2 | 1.58 |
| 2 | + | − | 2.6 | 0.0 | 0.08 |
| 3 | − | + | 2.6 | 0.0 | 0.09 |
| 4 | − | − | 2.6 | 0.0 | 0.08 |
| 5 | + | + | 0.0 | 0.0 | 0.00 |
| 6 | − | + | 0.0 | 0.0 | 0.00 |

### Example 15

Effects of 3DG, and Inhibition of 3DG, on Collagen Crosslinking.

Collagen is present at high levels in skin. To this end, it was determined what effect 3DG has on collagen crosslinking.

Collagen I was incubated in the presence or absence of 3DG in vitro. Calf skin collagen type I (1.3 mg; Sigma) was incubated in 20 mM Na-phosphate buffer, pH 7.25, either alone, with 5 mM 3DG, or with 5 mM 3DG plus 10 mM arginine, in a total volume of 1 ml at 37° C. for 24 hours and then frozen and lyophilized. The residue was dissolved in 0.5 ml of 70% formic acid and cyanogen bromide was added (20:1, w/w). This solution was incubated at 30° C. for 18 hours. Samples were dialyzed against 0.125 M Tris, pH 6.8, containing 2% SDS and 2% glycerol, in dialysis tubing with a molecular weight cutoff of 10,000. The samples were all adjusted to a volume of 1 ml. The extent of collagen crosslinking was determined by applying equal volumes of sample and analyzing by SDS-PAGE electrophoresis (16.5% Tris-tricine gel), as determined by the effects of 3DG on the migration of collagen.

It was found that treatment of collagen with 3DG caused the collagen to migrate as if it had a higher molecular weight, which is indicative of crosslinking. The image of the silver-stained gel in FIG. 12 demonstrates that there are fewer high molecular bands in the groups containing collagen alone or collagen plus 3DG plus arginine. There are more high molecular weight bands in the group treated with 3DG, in the absence of a 3DG inhibitor. There appears to be more protein in the sample treated with 3DG alone. Because all three samples started with the same amount of protein, without being bound by theory, it can be concluded that during dialysis fewer peptides escaped from the 3DG treated sample because more crosslinks were produced and higher molecular weight proteins were retained. In other words, there appears to be less protein in the control and 3DG plus arginine groups, because smaller molecular peptides diff-used out during dialysis.

### Example 16

Localization of 3DG in Skin.

The invention as described in the present disclosure identifies for the first time the presence of 3DG in skin.

A mouse skin model was used. One centimeter (1 cm) squares of skin were prepared and subjected to extraction with perchloric acid. 3DG was measured as described above. Six mice were used and the average amount of 3DG detected in the skin was 1.46+/−0.3 microM. This value was substantially higher than the plasma concentrations of 3DG detected in the same animals (0.19+/−0.05 microM). These data, and the data described below in Example 17, indicate that the high levels of 3DG in the skin are due to production of 3DG in the skin.

### Example 17

Localization of Amadorase mRNA in Skin.

Although high levels of 3DG were found in skin (see Example 16), it was not known whether the 3DG was formed locally and whether skin had the ability to produce 3DG enzymatically. The presence of Amadorase mRNA was analyzed and was utilized as one measure of the ability of skin to produce the 3DG present in skin (see previous example).

PolyA+messenger RNA isolated from human kidney and skin was purchased from Stratagene. The mRNA was used in RT-PCR procedures. Using the published sequence for Amadorase [Delpierre, G. et al. Identification, cloning, and heterologous expression of a mammalian fructosamine-3-kinase. 2000. Diabetes 49(10): p.1627-34.; Szwergold, B. S. et al. Purification, sequencing and characterization of fructoseamine-3-kinase (FN3K): An enzyme potentially involved in the control of non-enzymatic glycosylation. (Abstract). 200 1. Diabetes 50 Suppl. (2): p.A167], a reverse primer to the 3′ terminal end of the gene (bp 930-912) was subjected to RT to create a cDNA template for PCR. This same primer was used along with a forward primer from the middle of the Amadorase gene (bp 412-431) to amplify the Amadorase gene from the cDNA template. The product of the PCR should be a 519 bp fragment. Human skin and kidney samples were subjected to RT-PCR and analyzed by agarose gel electrophoresis, as were controls which contained no cDNA templates.

The results demonstrate that skin does indeed express Amadorase mRNA. Subsequent expression of the protein would account for production of 3DG in skin. As expected, a 519 bp product was observed (see FIG. 13). Not only was the 519 bp fragment found in kidney (lane 1), it was also found in skin (lane 3). The 519 bp fragment was not detected in the groups which received no cDNA template (lanes 2 and 4).

### Example 18

Inhibition of 3DG by Inhibiting Amadorase mRNA and Protein.

3DG synthesis may be inhibited by inhibiting the components of the enzymatic pathway leading to its synthesis. This can be done in several ways. For example, the enzyme which leads to the synthesis of 3DG, called Amadorase herein (a fructosamine-3-kinase) can be inhibited from acting using a compound as described above, but it can also be inhibited by blocking the synthesis of its message or protein or by blocking the protein itself, other than with a compound, as described above. Amadorase mRNA and protein synthesis and function may be inhibited using compounds or molecules such as transcription or translation inhibitors, antibodies, antisense messages or oligonucleotides, or competitive inhibitors.

Nucleic Acid and Protein Sequences

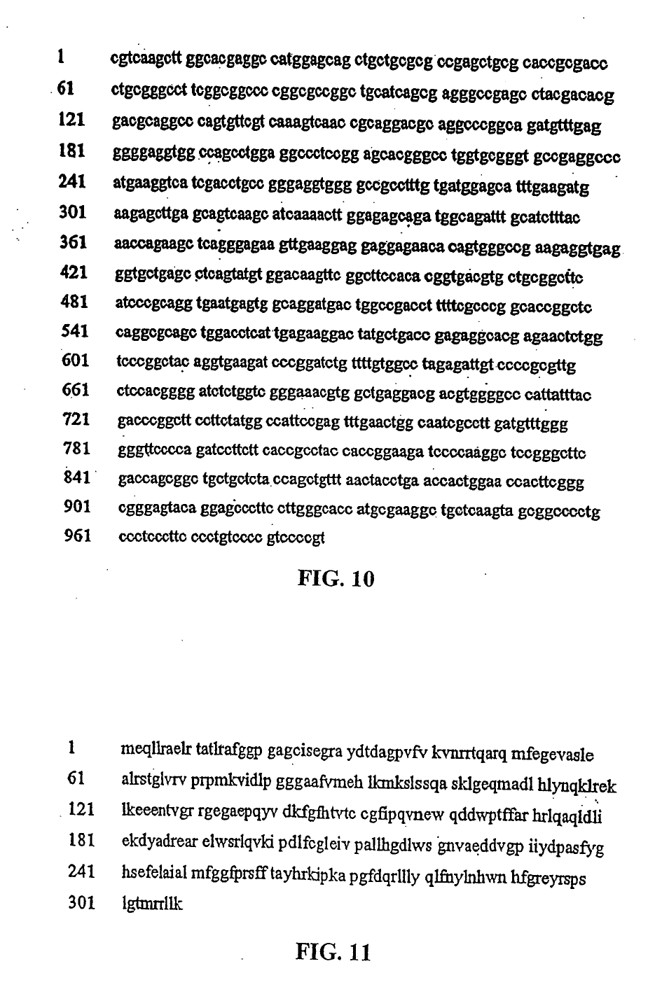

The following represents the 988 bp mRNA-derived DNA sequence for Amadorase (fructosamine-3-kinase), Accession No. NM_{—}022158 (SEQ ID NO: 1) (see FIG. 10):

| | |
|---|---|
| 1 | cgtcaagctt ggcacgaggc catggagcag ctgctgcgog ccgagctgcg caccgcgacc |
| 61 | ctgcgggcct tcggoggccc cggcgccggc tgcatcagcg agggccgagc ctacgacacg |
| 121 | gacgcaggcc cagtgttcgt caaagtcaac cgcaggacgc aggcccggca gatgtttgag |
| 181 | ggggaggtgg ccagcctgga ggccctccgg agcacgggcc tggtgcgggt gccgaggccc |
| 241 | atgaaggtoa tcgacctgcc gggaggtggg gccgcctttg tgatggagca tttgaagatg |
| 301 | aagagcttga gcagtcaagc atcaaaactt ggagagcaga tggcagattt gcatctttac |
| 361 | aaccagaagc tcagggagaa gttgaaggag gaggagaaca cagtgggccg aagaggtgag |
| 421 | ggtgctgagc otcagtatgt ggacaagttc ggcttccaca cggtgacgtg ctgcggcttc |
| 481 | atcccgcagg tgaatgagtg gcaggatgac tggccgacct ttttcgcccg gcaccggctc |
| 541 | caggcgcagc tggacctcat tgagaaggac tatgctgacc gagaggcacg agaactctgg |
| 601 | tcccggctac aggtgaagat cccggatctg ttttgtggcc tagagattgt ccccgcgttg |
| 661 | ctccacgggg atctctggtc gggaaacgtg gctgaggacg acgtggggcc cattatttac |
| 721 | gacccggctt ccttctatgg ccattccgag tttgaactgg caatcgcctt gatgtttggg |
| 781 | gggttcccca gatccttctt caccgcctac caccggaaga tccccaaggc tccgggcttc |
| 841 | gaccagcggc tgctgctcta ccagctgttt aactacctga accactggaa ccacttcggg |
| 901 | cgggagtaca ggagcccttc cttgggcacc atgcgaaggc tgctcaagta gcggcccctg |
| 961 | ccctcccttc ccctgtcccc gtccccgt |

The following represents the 309 amino acid residue sequence of human Amadorase (fructosamine-3-kinase), Accession No. NP_{—}071441 (SEQ ID NO:2) (see FIG. 11):

| | |
|---|---|
| 1 | meqllraelr tatlrafggp gagcisegra ydtdagpvfv kvnrrtqarq mfegevasle |
| 61 | alrstglvrv prpmkvidlp gggaafvmeh lkmkslssqa sklgeqmadl hlynqklrek |
| 121 | lkeeentvgr rgegaepqyv dkfgfhtvtc cgfipqvnew qddwptffar hrlqaqldli |
| 181 | ekdyadrear elwsrlqvki pdlfcgleiv pallhgdlws gnvaeddvgp iiydpasfyg |
| 241 | hsefelaial mfggfprsff tayhrkipka pgfdqrllly qlfhylnhwn hfgreyrsps |
| 301 | lgtmrrllk |

The sequences identified above were submitted by Delpierre et al. [Delpierre, G. et al. Identification, cloning, and heterologous expression of a mammalian fructosamine-3-kinase. 2000. Diabetes 49(10): p.1627-34.]. The sequence data of Szwergold et al. [Szwergold, B. S. et al. Purification, sequencing and characterization of fructoseamine-3-kinase (N73K): An enzyme potentially involved in the control of non-enzymatic glycosylation. (Abstract). 2001. Diabetes 50 Suppl. (2): p.A167] are in excellent agreement with those of Delpierre et al. in 307 of 309 amino acid residues.

### Example 19

Presence of Alpha-dicarbonyl Sugars in Sweat.

As disclosed herein, alpha-dicarbonyl sugars are present in skin, but their presence in sweat had not been determined. One of the functions of skin is to act as an excretory organ, therefore, it was determined whether alpha-dicarbonyl sugars are excreted in sweat.

Samples of human sweat were analyzed for the presence of 3DG, as described above. Samples from four subjects were obtained and 3DG was determined to be present at levels of 0.189, 2.8, 0.312, and 0.11 uM, respectively. Therefore, the results demonstrate the presence of 3DG in sweat.

### Example 20

Effects of DYN 12 (3-O-methylsorbitollysine) on Skin Elasticity.

Administration of DYN 12, a small molecule inhibitor of Amadorase, reduces 3DG levels in the plasma of diabetic and non-diabetic animals [Kappler, F., Su, B., Schwartz, M L, Tobia, A M, and, Brown, T. DYN]12, a small molecule inhibitor of the enzyme Amadorase, lowers 3-deoxyglucosone levels in diabetic rats. 2002. Diabetes Technol. Ther. Winter 3(4): p.609-606].

Experiments were performed to determine the effects of DYN 12 on the loss of skin elasticity associated with diabetes. To this end, two groups of STZ-diabetic rats and two groups of normal rats were subjected to treatment with DYN 12 or saline. One 'group of STZ-diabetic rats (n=9) received daily subcutaneous injections of DYN 12 at 50 mg/kg for eight weeks, as did one group of normal rats (n=6). A group of control diabetic rats (n=10) and a group of normal rats (n=6) received saline instead of DYN 12. One rat was removed from the diabetic DYN 12 group after 2 weeks because its blood glucose readings were inconsistent (too low) with other diabetic rats.

A non-invasive procedure based on CyberDERM, Inc. technology utilizing a skin elasticity measurement device was used to test the effects of DYN 12 treatment on skin elasticity. The procedure provides for non-invasive measurement of skin elasticity based upon the amount of vacuum pull required to displace skin. A suction cup probe is adhered to an area of shaved skin in order to form an airtight seal. Then, a vacuum is applied to the area of the skin inside the suction cup until the skin is displaced past a sensor located inside the probe. Accordingly, the more pressure that is required to displace the skin, the less elastic the skin is.

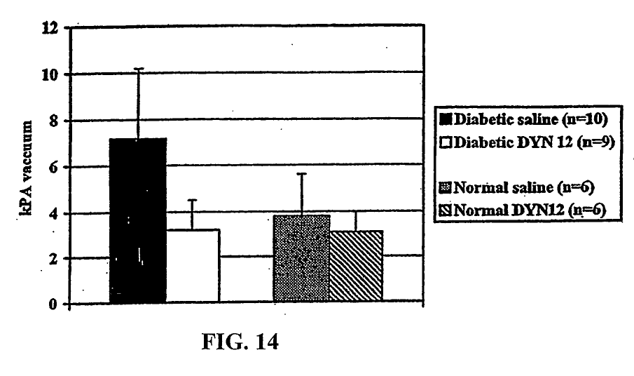

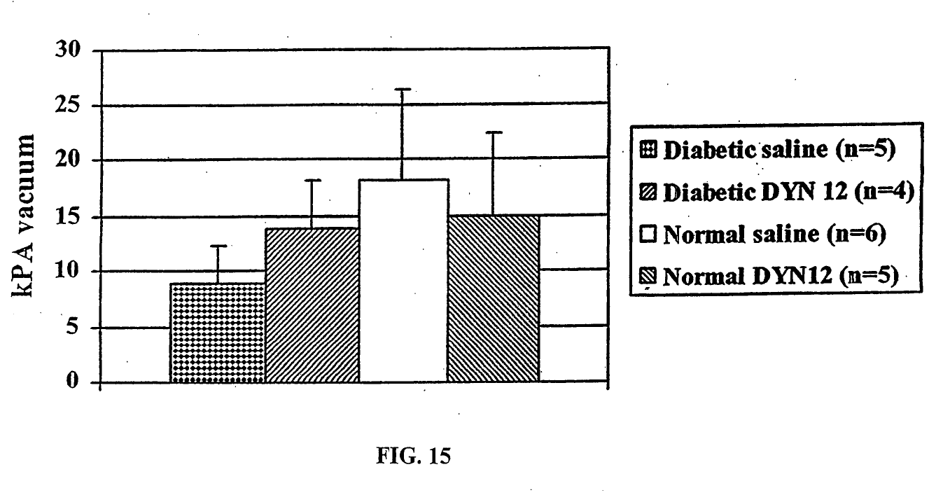

The data demonstrate that after eight weeks of treatment skin elasticity in diabetic rats treated with DYN 12 was greater than skin elasticity in diabetic animals which were treated with saline. As seen in FIG. 14, the amount of pressure needed to displace the skin of diabetic rats treated with saline (7.2+/−3.0 kPA) was approximately 2 to 2.25 fold higher than the pressure needed to displace the skin of diabetic animals treated with DYN 12 (3.2+/−1.2 kPA). Also, the elasticity value observed in diabetic rats treated with DYN 12 was not statistically different from the value found in non-diabetic rats treated with saline (p=0.39) (Table E). Thus, the result of treatment of diabetic animals with DYN 12, an indirect inhibitor of 3DG, was skin with-greater elasticity than skin in diabetic animals which received only saline.

TABLE E
Statistical Analysis and Comparison of Cohort Groups.

| Group 1 | Group 2 | p value |
|---|---|---|
| Diabetic saline | Non-diabetic saline | p = 0.01 |
| Diabetic saline | Diabetic DYN 12 | p = 0.001 |
| Diabetic saline | Non-diabetic DYN 12 | p = 0.003 |
| Diabetic DYN 12 | Non-diabetic DYN 12 | p = 0.39 |
| Diabetic DYN 12 | Non-diabetic saline | p = 0.26 |
| Non-diabetic saline | Non-diabetic DYN 12 | p = 0.20 |

The above data demonstrate that the administration of DYN 12 to diabetic rats prevents the loss of skin elasticity (e.g., sclerosis and thickening of the basement membrane of the skin) that is typically observed in untreated diabetic rats, which is evidence that the excess 3DG found in diabetics is the cause of the loss of elasticity. The data disclosed herein further indicate that reducing 3DG levels can also serve to maintain skin elasticity in normal individuals.

Skin elasticity measurements were also taken on the test subjects as described above, but without sedating the test animals before measurement. FIG. 15 illustrates skin elasticity measurements taken on the hind leg of the test subjects while the subjects were alert and being restrained by a technician.

In these experiments, the animals were fiercely fighting restraint and the results are different. The diabetic animals without drug treatment showed less ability to “pull away” from the suction cup and therefore show less “resistance to pull”. On the other hand, both the diabetic animals receiving drug and the normal animals had a greater capacity to pull away from the suction cup, and both groups of animals demonstrated stiffness and greater muscle tension. This indicates that the inhibition of the enzyme, and most likely, inactivation of 3DG, results in the sparing of microcirculation deterioration and neuro-deterioration that typifies the diabetic condition.

### Example 21

Level of 3DG in Scleroderma Skin.

It has been determined, according to the methods disclosed previously elsewhere herein, that normal skin had the following concentrations of 3DG (data from several subjects): 0.9 μM, 0.7 μM, and 0.6 μM. Several samples of skin from several scleroderma patients were similarly assayed and had the following level of 3DG: 15 μM, 130 μM, and 3.5 μM. Accordingly, these data demonstrate that the level of 3DG in the skin of scleroderma patients is significantly elevated compared with the level of 3DG in the skin of normal humans.

### Example 22

mRNA for Collagen Type 1 is Down Regulated Following Administration of Fructoselysine, the Substrate for Amadorase, to Scleroderma Cells.

In these experiments 5 mM FL was added to cultured human dermal fibroblasts acquired from a patient with scleroderma. After 72 hours, cells were collected and mRNA isolated. Equal amounts of each mRNA preparation was separated by electrophoresis and the amount of collagen 1A1 mRNA detected by Northern blot using a radioactive probe to the collagen 1A1 mRNA as shown in FIG. 19. A phosphoimager was used to quantify the amount of collagen type 1 mRNA. A radioactive probe to the GAPDH mRNA was used as a control to normalize the level of collagen 1A1 mRNA in each sample. The level of collagen 1A1 RNA was decreased by 40%.

These data demonstrate that the Amadorase pathway can downregulate the amount of type I collagen mRNA produced.

### Example 23

Amadorase Activity is Inhibited by Copper in a Concentration Dependent Manner.

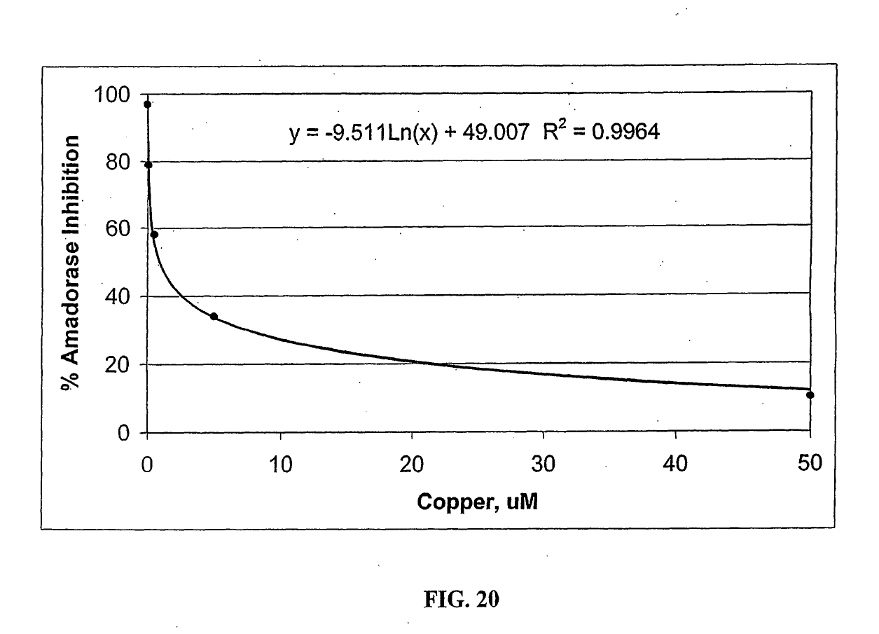

Experiments were performed to test the effect of copper on the activity of the Amadorase enzyme in vitro. Using methods described elsewhere, increasing amounts of copper in the form of CuSO₄, was added to an in vitro assay. Purified Amadorase was added to the reaction, incubated at 37° C. for 15 minutes, and the amount of FL3P measured. The graph depicted in FIG. 20 represents percent Amadorase activity as a function of copper concentration. Copper sulfate inhibits Amadorase by 50% at a concentration of about 1 uM.

### Example 24

Suppression of Collagen Production.

Collagen type I production is suppressed by approximately 40% following administration of 3 mM fructoselysine, the substrate for Amadorase, to human dermal fibroblasts. Conversely, administration of 3 mM DYN 12 (3-O-methylsorbitollysine), an Amadorase inhibitor, increased collagen type I production by 50%.

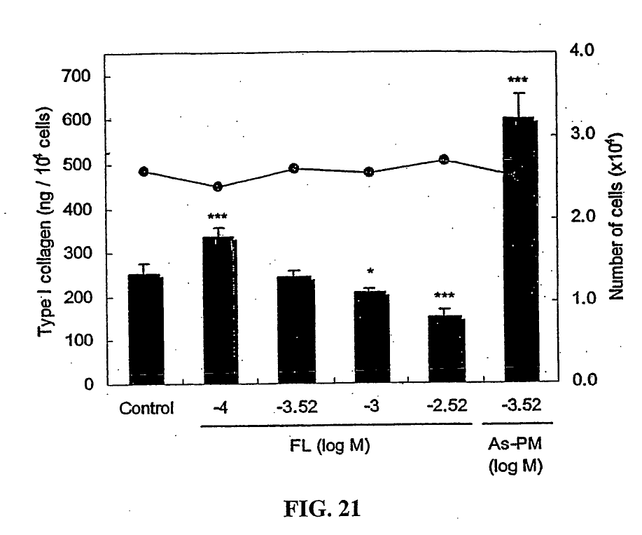

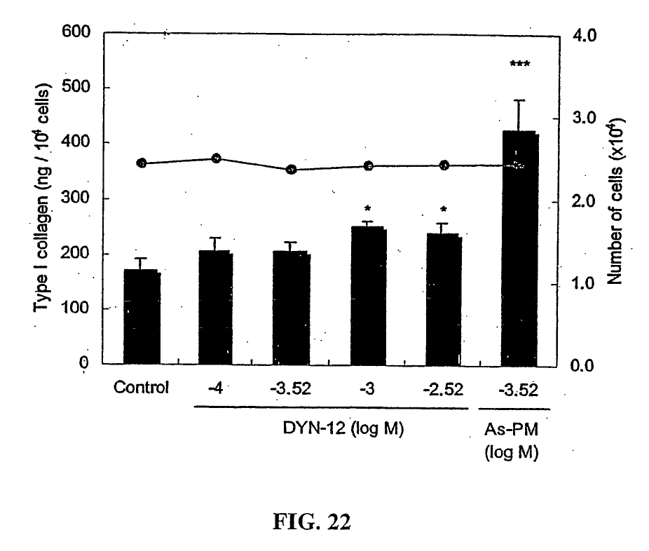

In these experiments FL or DYN 12 was added to cultured human dermal fibroblasts acquired from a 66 year old female. After 72 hours, the concentration of the type 1 collagen (procollagen type I C-peptide) in the supernatant was measured using EIA. Percent changes are relative to controls cultures with no additions. These data (FIG. 21) demonstrate that the Amadorase pathway can affect the production of type I collagen. Increasing activity of the pathway by FL decreases collagen type I while inhibiting the pathway using DYN 12 has the exact opposite effect (FIG. 22).

### Example 25

Desmosine Analysis.

For desmosine analysis, the biopsy of tissue is fixated using paraffin. The paraffin is removed from the secretions in the microfuge tubes by incubating for 10 minutes with 500 μL of xylene. Five microliters of water is added, the tubes gently vortexed and then microfuged. The xylene is carefully removed, 400 μL of 6N HCl is added to the protein pellet, and the samples hydrolyzed for 24 hours at 100° C. The acid is evaporated in a savant vacuum centrifuge and the hydrolysate redissolved in 400 μL distilled water. The samples are vortexed and microfuged and 20 μL removed from each tube for desmosine analysis by radioimmunoassay. Protein content is determined in 2 μL of the hydrolysate by slightly modifying the ninhydrin method. A stock ninhydrin solution is made by dissolving 10 g of ninhydrin in 375 mL of ethylene glycol and 125 mL of 4N sodium acetate buffer, pH 5.5. For the working solution, 250 μL of 10% stannous chloride suspension is added for every 10 mL of the ninhydrin stock solution. Hydroxyproline is determined in 50 μL of hydrolysate by amino acid analysis.

### Example 26

Regulation of Desmosines Via the Amadorase Pathway.

The production of desmosines, a precursor to elastin, can be regulated by compounds that affect the Amadorase pathway. Skin from normal, diabetic, and animals treated with 1-deoxy-1-morpholinofructose were excised and for desmosine content as described in example 29. The results demonstrated that diabetic animals had greater levels of desmosines than non-diabetic animals (P=0.00034) and that diabetic animals treated with 1 deoxy-1-morpholinofructose had lower levels of desmosines compared to untreated diabetic animals (P=0.00242).

| | | | |
|---|---|---|---|
| Rat no. | pmD/mgP | Rat no. | pmD/mgP |

| | |
|---|---|
| Diabetic rats | Normal rats |

| | | | |
|---|---|---|---|
| 1 | 88.7 | 9 | 56.3 |
| 2 | 87.4 | 10 | 61.3 |
| 3 | 69.3 | 11 | 62.1 |
| 4 | 93.3 | 12 | 61.6 |
| 5 | 88.2 | 13 | 40.9 |
| 6 | 81.4 | 14 | 49 |
| 7 | 79.4 | | |
| 8 | 77.8 | | |

| | |
|---|---|
| | Treated |
| Untreated | diabetic rats |
| diabetic rats | 1deoxy-1- |
| Saline | morpholinofructose |

| | | | |
|---|---|---|---|
| 1 | 88.7 | 15 | 63.1 |
| 2 | 87.4 | 16 | 67.9 |
| 3 | 69.3 | 17 | 67.9 |
| 4 | 93.9 | 18 | 88.1 |
| 5 | 88.2 | 19 | 64.3 |
| 6 | 81.4 | 20 | 65.8 |
| 7 | 79.4 | 21 | 65.1 |
| 8 | 77.8 | 22 | 66.8 |

### Example 27

The Production of Desmosines, a Precursor to Elastin, can be Regulated by Compounds that Affect the Amadorase Pathway—Sample Collection.

Lung tissues from mice made diabetic with STZ was excised and analyzed for desmosine levels. The levels of desmosines in diabetic mice were greater than non-diabetic mice. The levels of desmosines in mice treated with meglumine were less than mice not so treated, regardless of whether the mice were diabetic.

### Example 28

The Production of Desmosines, a Precursor to Elastin, can be Regulated by Compounds that Affect the Amadorase Pathway.

Aorta samples from diabetic rats treated with 1-deoxy-1-morpholinofructose showed a decrease in desmosine levels compared to untreated diabetic rats (P=0.104).

| | |
|---|---|
| Untreated | Treated diabetic rats |
| diabetic rats | 1-deoxy-1- |
| Saline | morpholinofructose |

| Rat no. | pmD/mgP | Rat no. | pmD/mgP |
|---|---|---|---|
| A144 | 4650 | A175 | 2558 |
| A145 | 3206 | A176 | 2842 |
| A146 | 3429 | A181 | 2906 |
| A147 | 3411 | A182 | 3633 |
| A149 | 3834 | A183 | 3681 |
| A150 | 3402 | A184 | 3809 |
| A151 | 3341 | | |
| A152 | 3793 | | |
| A153 | 4169 | | |

### Example 29

Distribution of DYN-12

Rats were injected intraperitoneally with 1 ml of a 100 mM solution of DYN-12 (100 micromols). Urine was collected for 1 hr, and DYN-12 levels measured. After 1 hr the animals were sacrificed and DYN-12 levels measured in both plasma and kidney tissue.

| | DYN-12 concentration | | |
|---|---|---|---|
| | Plasma | Urine | Kidney |
| | (micromole/ml) | (micromol/ml) | (micromol/g) |
| Animal #1 | 0.335 | 41.99 | 6.54 |
| Animal #2 | 0.269 | 17.81 | 9.19 |
| Animal #3 | 0.296 | 15.03 | 8.21 |

This illustrates that after 1 hr, DYN-12 is present at very low levels in plasma, excreted in urine, and in kidneys at levels 2-3x higher than the Ki for fructoseamine kinase inhibition (2-3 mM).

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

The disclosures of each and every patent, patent application, and publication cited herein are hereby incorporated herein by reference in their entirety.

1. A method of decreasing desmosine levels in a mammal in need thereof, said method comprising administering to said mammal a composition comprising an inhibitor of the Amadorase pathway.

2. The method of claim 1, wherein said composition comprises an inhibitor of fructoseamine kinase.

3. The method of claim 1, wherein said composition further comprises an inhibitor of 3DG.

4. The method of claim 1, wherein said mammal is a human.

5. The method of claim 4, wherein said human has at least one disease selected from the group consisting of diabetes and lung fibrosis.

6. A method of stabilizing desmosine levels in a mammal in need thereof, said method comprising administering to said mammal a composition comprising an inhibitor of the Amadorase pathway.

7. The method of claim 6, wherein said composition comprises an inhibitor of fructoseamine kinase.

8. The method of claim 6, wherein said composition further comprises an inhibitor of 3DG.

9. The method of claim 6, wherein said mammal is a human.

10. The method of claim 9, wherein said human has at least one disease selected from the group consisting of diabetes and lung fibrosis.

11. The method of claim 1, wherein said desmosine levels are in at least one of the locations selected from the group consisting of the extracellular matrix, lung, kidney, skin, heart, arteries, ligament and elastic cartilage.

12. The method of claim 2, wherein said inhibitor of fructoseamine kinase is administered to said mammal via a route selected from the group consisting of topical, oral, rectal, vaginal, intramuscular, subcutaneous, and intravenous.

13. The method of claim 2, wherein said inhibitor of fructoseamine kinase is an antibody.

14. The method of claim 2, wherein said fructoseamine kinase is encoded by a nucleic acid comprising a nucleic acid encoding the amino acid sequence set forth in SEQ ID NO:2.

15. A method of decreasing desmosine levels in a mammal in need thereof, said method comprising administering to said mammal a composition comprising an inhibitor of the Amadorase pathway, wherein said inhibitor is a compound comprising the formula of formula XIX: 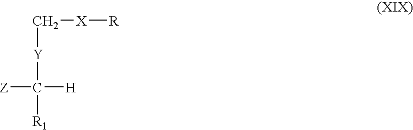
a. wherein X is —NR′—, —S(O)—, —S(O)2—, or —O—, R′ being selected from the group consisting of H, linear or branched chain alkyl group (C1-C4), CH2(CHOR2)nCH2OR2 where n=1-5 and R2 is H, alkyl (C1-C4) or an unsubstituted or substituted aryl group (C6-C10) or araalkyl group (C7-C10), CH(CH2OR2)(CHOR2)nCH2OR2 where n=1-4 and R2 is H, alkyl (C1-C4) or an unsubstituted or substituted aryl group (C6-C10) or araalkyl group (C7-C10), an unsubstituted or substituted aryl group (C6-C10), and an unsubstituted or substituted aralkyl group (C7-C10);
b. R is a substituent selected from the group consisting of H, an amino acid residue, a polyaminoacid residue, a peptide chain, a linear or branched chain aliphatic group (C1-C8), which is unsubstituted or substituted with at least one nitrogen- or oxygen-containing substituent, a linear or branched chain aliphatic group (C1-C8), which is unsubstituted or substituted with at least one nitrogen—or oxygen-containing substituent and interrupted by at least one —O—, —NH—, or —nNR″ —moiety;
c. R″ being linear or branched chain alkyl group (C1-C6) and an unsubstituted or substituted aryl group (C6-C 10) or aralkyl group (C7-C 10), with the proviso that when X represents —NR′—, R and R′, together with the nitrogen atom to which they are attached, may also represent a substituted or unsubstituted heterocyclic ring having from 5 to 7 ring atoms, with at least one of nitrogen and oxygen being the only heteroatoms in said ring, said aryl group (C6-C10) or aralkyl group (C7-C10) and said heterocyclic ring substituents being selected from the group consisting of H, alkyl (C1-C6), halogen, CF3, CN, NO2 and —O—alkyl (C1-C6); R1 is a polyol moiety having 1 to 4 linear carbon atoms, Y is a hydroxymethylene moiety —CHOH—; Z is selected from the group consisting of —H, —O—alkyl (C1-C6), —halogen —CF3, —CN, —COOH, and —SO3H2, and optionally —OH;
d. The isomers and pharmaceutically acceptable salts of said compound, except that X-R in the above formula does not represent hydroxyl or thiol.

16. The method of claim 15, wherein said composition comprises said inhibitor from about 0.0001% to about 15% by weight.

17. The method of claim 16, further wherin said composition is a pharmaceutical composition.

18. The method of claim 15, wherein said compound comprising formula XIX is selected from the group consisting of galactitol lysine, 3-deoxy sorbitol lysine, 3-deoxy-3-fluoro-xylitol lysine, 3-deoxy-3-cyano sorbitol lysine, 3-O-methyl sorbitollysine, meglumine, sorbitol lysine and mannitol lysine.

19. The method of claim 15, wherein said compound is 3-O-methyl sorbitollysine.

20. A method of decreasing the level of mRNA for collagen in a mammal by increasing the flux through the Amadori pathway in said mammal, said method comprising administering to said mammal a compound comprising formula XIX(b): 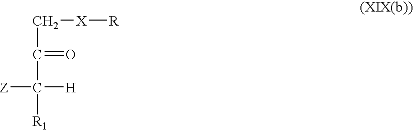
a. wherein X is —NR′—, —S(O)—, —S(O)2-, or —O—, R′ being selected from the group consisting of H or a guanidine group, linear or branched chain alkyl group (C1-C4), CH2(CHOR2)nCH2OR2 where n=1-5 and R2 is H, alkyl (C1-C4) or an unsubstituted or substituted aryl group (C6-C10) or araalkyl group (C7-C10), CH(CH2OR2)(CHOR2)nCH2OR2 where n=1-4 and R2 is H, alkyl (C1-C4) or an unsubstituted or substituted aryl group (C6-C10) or araalkyl group (C7-C10), an unsubstituted or substituted aryl group (C6-C10), and an unsubstituted or substituted aralkyl group (C7-C10);
b. R is a substituent selected from the group consisting of H, an amino acid residue, a polyaminoacid residue, a peptide chain, a linear or branched chain aliphatic group (C1-C8), which is unsubstituted or substituted with at least one nitrogen- or oxygen-containing substituent, a linear or branched chain aliphatic group (C1-C8), which is unsubstituted or substituted with at least one nitrogen- or oxygen-containing substituent and interrupted by at least one —O—, —NH—, or —NR″— moiety;
c. R″ being linear or branched chain alkyl group (C1 -C6) and an unsubstituted or substituted aryl group (C6-C10) or aralkyl group (C7-C10), with the proviso that when X represents —NR′—, R and R′, together with the nitrogen atom to which they are attached, may also represent a substituted or unsubstituted heterocyclic ring having from 5 to 7 ring atoms, with at least one of nitrogen and oxygen being the only heteroatoms in said ring, said aryl group (C6-C10) or aralkyl group (C7-C10) and said heterocyclic ring substituents being selected from the group consisting of H, alkyl (C1-C6), halogen, CF3, CN, NO2 and —O-alkyl (C1-C6); R1 is a polyol moiety having 1 to 4 linear carbon atoms, Z is selected from the group consisting of —H, —O-alkyl (C1-C6), —halogen —CF3, —CN, —COOH, and —SO3H2, and optionally —OH;
d. the isomers and pharmaceutically acceptable salts of said compound, except that X-R in the above formula does not represent hydroxyl or thiol.

20. The method of claim 20, wherein said collagen is Type I collagen.

21. The method of claim 20, wherein said compound is a substrate for fructoseamine kinase.

22. The method of claim 20, wherein said compound is fructoselysine.

23. A method of treating scleroderma in a mammal, said method comprising administering to said mammal a composition comprising a compound that increases the flux through the Amadorase Pathway in said mammal, thereby decreasing the levels of mRNA for collagen Type I.

24. A method of treating keloids in a mammal, said method comprising administering to said mammal a composition comprising a compound that increases the flux through the Amadorase Pathway in said mammal, thereby decreasing the levels of mRNA for collagen Type I.

26. The method of claim 24, wherein said compound stimulates fructoseamine kinase.

27. The method of claim 24, wherein said compound is selected from the group consisting of fructose lysine 3 phosphate and an analog of fructose lysine 3 phosphate.

28. A method of treating scleroderma in a mammal, said method comprising the administration to said mammal of a composition comprising:
a) a first compound that stimulates the flux through the Amad\dot{o}rase pathway; and
b) a second compound that inactivates 3DG.

29. The method of claim 17.4, wherein said second compound is structural formula I: 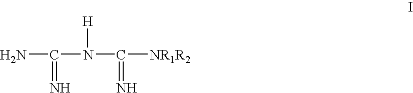
wherein R1 and R2 are independently selected from the group consisting of a hydrogen, a lower alkyl, a lower alkoxy and an arly group; or wherein said R1 said R2 together with a nitrogen atom form a heterocyclic ring containing from 1 to 2 heteroatoms and 2 to 6 carbon atoms, the second of said hereoatoms comprising nitrogen, oxygen, or sulfur; further wherein said lower alkyl group is selected from the group consisting of 1 to 6 carbon atoms; wherein said lower alkoxy group is selected from the group consisting of 1 to 6 carbon atoms; and wherein said aryl group comprises substituted and unsubstituted phenyl and pyridyl groups.

30. A method of inhibiting the reaction of at least one dicarbonyl compound with tropoelastin in a mammal, said method comprising administering to said mammal an effective amount of an inhibitor of an alpha-dicarbonyl sugar function.

31. The method of claim 30, wherein said dicarbonyl compound is 3DG.

32. The method of claim 30, wherein said inhibitor chelates 3DG.

33. The method of claim 30, wherein said inhibitor detoxifies 3DG.

34. The method of claim 31, wherein said inhibitor is selected from the group consisting of structural formulas I-XVII and XVIII.

35. The method of claim 30, wherein said inhibitor is structural formula I: 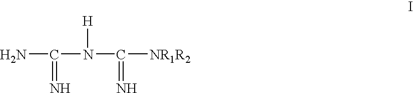
wherein R1 and R2 are independently selected from the group consisting of a hydrogen, a lower alkyl, a lower alkoxy and an aryl group; or wherein said R1 and said R2 together with a nitrogen atom form a heterocyclic ring containing from 1 to 2 heteroatoms and 2 to 6 carbon atoms, the second of said heteroatoms comprising nitrogen, oxygen, or sulfur; further wherein said lower alkyl group is selected from the group consisting of 1 to 6 carbon atoms; wherein said lower alkoxy group is selected from the group consisting of 1 to 6 carbon atoms; and wherein said aryl group comprises substituted and unsubstituted phenyl and pyridyl groups.

36. The method of claim 30, wherein said compound is selected from the group consisting of N, N-dimethylimidodicarbonimidic diamide, imidodicarbonimidic diamide, N-phenylimidodicarbonimidic diamide, N-(aminoiminomethyl)-4-morpholinecarboximidamide, N-(aminoiminomethyl)-4-thiomorpholinecarboximidamide, N-(aminoiminomethyl)-4-methyl-1-piperazinecarboximidamide, N-(aminoiminomethyl)-1-piperidinecarboximidamide, N-(aminoiminomethyl)-1-pyrrolidinecarboximidamide, N-(aminoiminomethyl) -I-hexahydroazepinecarboximidamide, (aminoiminomethyl)-I-hexahydroazepinecarboximidamide, N-4-pyridylimidodicarbonimidic diamide, N, N-di-n-hexylimidodicarbonimidic diamide, N,N-di-n-pentylimidodicarbonimidic diamide, N,N-d-n-butylimidodicarbonimidic diamide, N,N-dipropylimidodicarbonimidic diamide, and N,N-diethylimidodicarbonimidic diamide.

37. The method of claim 30 wherein said structural formula is structural formula II: 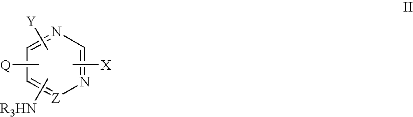
wherein Z is N or CH; wherein X, Y, and Q each independently is selected from the group consisting of a hydrogen, an amino, a heterocyclo, an amino lower alkyl, a lower alkyl, and a hydroxy group; further wherein R3 comprises a hydrogen or an amino group or their corresponding 3-oxides; wherein said lower alkyl group is selected from the group consisting of 1 to 6 carbon atoms; wherein said heterocyclic group is selected from the group consisting of 3 to 6 carbon atoms; and wherein X, Y, and Q can each be present as a hydroxy variant on a nitrogen atom.

38. The method of claim 37, wherein said compound is selected from the group consisting of 4,5-diaminopyrimidine, 4-amino-5-aminomethyl-2-methylpyrimidine, 6-(piperidino)-2,4-diaminopyrimidine 3-oxide, 4,6-diaminopyrimidine, 4,5,6-triaminopyrimidine, 4,5-diamino-6-hydroxy pyrimidine, 2,4,5-triamino-6-hydroxypyrimidine, 2,4,6-triaminopyrimidine, 4,5-diamino-2-methylpyrimidine, 4,5-diamino-2,6-dimethylpyrimidine, 4,5-diamino-2-hydroxy-pyrimidine, and 4,5-diamino-2-hydroxy-6-methylpyrimidine.

39. The method of claim 30, wherein said structural formula is structural formula III: 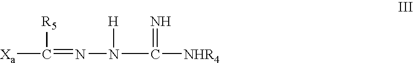
wherein R4 is hydrogen or acyl, R5 is hydrogen or lower alkyl, Xa is a substituent selected from the group consisting of a lower alkyl, a carboxy, a carboxymethyl, an optionally substituted phenyl and an optionally substituted pyridyl group, wherein said optional substituent is selected from the group consisting of a halogen, a lower alkyl, a hydroxy lower alkyl, a hydroxy, and an acetylamino group; further wherein, when X is a phenyl or pyridyl group, optionally substituted, R5 is hydrogen; and wherein, said lower alkyl group is selected from the group consisting of 1 to 6 carbon atoms.

40. The method of claim 39, wherein said compound is selected from the group consisting of N-acetyl-2-(phenylmethylene)hydrazinecarboximidamide, 2-(phenylmethylene)hydrazinecarboximidamide, 2-(2,6-dichlorophenylmethylene) hydrazinecarboximidamide pyridoxal guanylhydrazone, pyridoxal phosphate guanylhydrazone, 2-(1-methylethylidene)hydrazinecarboximidamide, pyruvic acid guanylhydrazone, 4-acetamidobenzaldehyde guanylhydrazone, 4-acetamidobenzaldehyde N-acetylguanylhydrazone, and acetoacetic acid guanylhydrazone.

41. The method of claim 30, wherein said structural formula is structural formula IV: 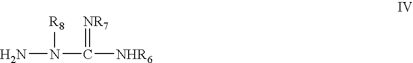
wherein, R6 is selected from the group consisting of a hydrogen, a lower alkyl group, and a phenyl group, further wherein said phenyl group is optionally substituted by a structure selected from the group consisting of a 1-3 halo, an amino, a hydroxy, and a lower alkyl group, wherein when said phenyl group is substituted, a point of said substitution is selected from the group consisting of an ortho, a meta, and a para point of attachment of said phenyl ring to a straight chain of said structural formula IV; R7 is selected from the group consisting of a hydrogen, a lower alkyl group, and an amino group; R8 is hydrogen or a lower alkyl group; further wherein said lower alkyl group is selected from a lower alkyl group consisting of 1 to 6 carbon atoms.

42. The method of claim 41, wherein said compound is selected from the group consisting of equival n-butanehydrazonic acid hydrazide, 4-methylbenzamidrazone, N-methylbenzenecarboximidic acid hydrazide, benzenecarboximidic acid 1-methylhydrazide, 3-chlorobenzamidrazone, 4-chlorobenzamidrazone, 2-fluorobenzamidrazone, 3-fluorobenzamidrazone, 4-fluorobenzamidrazone, 2-hydroxybenzamidrazone, 3-hydroxybenzamidrazone, 4-hydroxybenzamidrazone, 2-aminobenzamidrazone, benzenecarbohydrazonic acid hydrazide, and benzenecarbohydrazonic acid 1-methylhydrazide.

43. The method of claim 30, wherein said structural formula is structural formula V: 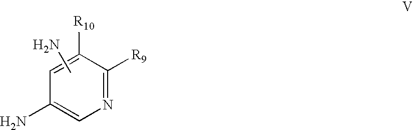
wherein R9 and R10 are independently selected from the group consisting of a hydrogen, a hydroxy, a lower alkyl, and a lower alkoxy, further wherein a “floating” amino group is adjacent to a fixed amino group; said lower alkyl group is selected from a lower alkyl group consisting of 1 to 6 carbon atoms; and said lower alkoxy group is selected from a lower alkoxy group consisting of 1 to 6 carbon atoms.

44. The method of claim 43, wherein said compound is selected from the group consisting of 3,4-diaminopyridine, 2,3-diaminopyridine, 5-methyl-2,3-diaminopyridine, 4-methyl-2,3 -diaminopyridine, 6-methyl-2,3 -pyridinediamine, 4,6-dimethyl-2,3-pyridinediamine, 6-hydroxy-2,3-diaminopyridine, 6-ethoxy-2,3-diaminopyridine, 6-dimethylamino-2,3-diaminopyridine, diethyl 2-(2,3-diamino-6-pyridyl) malonate, 6 (4-methyl-1-pyperazinyl)-2,3-pyridinediamine, 6-(methylthio)-5 (trifluoromethyl)-2,3-pyridinediamine, 5-(trifluoromethyl)-2,3-pyridinediamine, 6-(2,2,2-trifluorethoxy)-5- (trifluoromethyl)-2,3-pyridinediamine, 6-chloro-5-(trifluoromethyl)-2,3-pyridinediamine, 5-methoxy-6-(methylthio)-2,3-pyridinediamine, 5-bromo-4-methyl-2,3-pyridinediamine, 5-(trifluoromethyl-2,3-pyridinediamine, 6-bromo-4-methyl-2,3-pyridinedlamine, 5-bromo-6-methyl-2,3-pyridinediamine, 6-methoxy-3,4-pyridinediamine, 2-methoxy-3,4-pyridinediamine, 5-methyl-3,4-pyridinediamin, 5-methoxy-3,4-pyridinediamine, 5-bromo-3,4-pyridinediamine, 2,3,4-pyridinetriamine, 2,3,5-pyridinetriamine, 4-methyl-2,3,6-pyridinetriamine, 4-(methylthio)-2,3,6-pyridinetriamine, 4-ethoxy-2,3,6-pyridinetriamine, 2,3,6-pyridinetriamine, 3,4,5-pyridinetriamine, 4-methoxy-2,3-pyridinediamine, 5-methoxy-2,3-pyridinediamine, and 6-methoxy-2,3-pyridinediamine.

45. The method of claim 30, wherein said structural formula is structural formula VI: 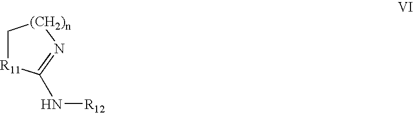
wherein n is 1 or 2, R 11is an amino group or a hydroxyethyl group, and R12 is selected from the group consisting of an amino group, a hydroxyalkylamino group, a lower alkyl group, and a group of the formula alk-Ya, further wherein alk is a lower alkylene group and Ya is selected from the group consisting of a hydroxy, a lower alkoxy group, a lower alkylthio group, a lower alkylamino group, and a heterocyclic group, wherein said heterocyclic group contains 4 to 7 ring members and 1 to 3 heteroatoms; further wherein, when said R11 is a hydroxyethyl group then said R12 is an amino group; said lower alkyl group is selected from the group consisting of 1 to 6 carbon atoms, said lower alkylene group is selected from the group consisting of 1 to 6 carbon atoms, and said lower alkoxy group is selected from the group consisting of 1 to 6 carbon atoms.

46. The method of claim 45, wherein said compound is selected from the group consisting of 1-amino-2-[2-(2-hydroxyethyl) hydrazino]-2-imidazoline, 1-amino-[2-(2-hydroxyethyl) hydrazino]-2-imidazoline, 1-amino-2-(2-hydroxyethylamino)-2-imidazoline, 1-(2-hydroxyethyl)-2-hydrazino-1,4,5,6-tetrahydropyrimidine, 1-(2-hydroxyethyl) 2-hydrazino-2-imidazoline, I -amino-2-([2-(4-morpholino)ethyl]amino)imidazoline, ([2-(4-morpholino)ethyl]amino)imidazoline, 1 -amino-2-([3-(4-morpholino) propyl]amino)imidazoline, 1-amino-2-([3-(4-methylpiperazin-1-yl)propyl]-amino)imidazoline; 1-amino-2-([3-(dimethylamino)propyl] amino)imidazoline, 1-amino-2-[(3-ethoxypropyl)amino] imidazoline, 1-amino-2-([3-(1-imidazolyl)propyl] amino)imidazoline, 1-amino-2-(2-methoxyethylamino)-2-imidazoline, (2-methoxyethylamino)-2-imidazoline, 1-amino-2-(3-isopropoxypropylamino)-2-imidazoline, 1-amino-2-(3-methylthiopropylamino)-2-imidazoline, 1-amino-2 [3-(1-piperidino)propylamino)imidazoline, 1-amino-2-[2, 2-dimethyl-3-(dimethylamino) propylamino]-2-imidazoline, and 1-amino-2-(neopentylamino)-2-imidazoline.

47. The method of claim 30 wherein said structural formula is structural formula VII: 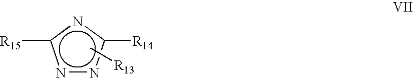
wherein, R13 is selected from the group consisting of a hydrogen and an amino group, R14 and R15 are independently selected from the group consisting of an amino group, a hydrazino group, a lower alkyl group, and an aryl group, further wherein, one of said R13, R14, and R15 must be an amino group or a hydrazino group; wherein said aryl group is selected from the group consisting of 6 to 10 carbon atoms, and said lower alkoxy group is selected from the group consisting of 1 to 6 carbon atoms.

48. The method of claim 47, wherein said compound is selected from the group consisting of 3,4-diamino-5-methyl-1,2,4-triazole, 3,5-dimethyl-4H-1,2,4-triazol-4-amine, 4-triazol-4-amine, 4-triazol-4-amine, 4-triazol-4-amine, 2,4-triazole-3,4-diamine, 5-(1-ethylpropyl)-4H-1,2,4-triazole-3,4-diamine, 5-isopropyl-4H-1,2,4-triazole-3,4-diamine, 5-cyclohexyl-4H-1,2,4-triazole-3,4-diamine, 5-methyl-4H-1,2,4-triazole-3,4-diamine, 5-phenyl-4H-1,2,4-triazole-3,4-diamine, 5-propyl-4H-1 ,2,4-triazole-3,4-diamine, and 5-cyclohexyl-4H-1,2,4-triazole-3,4-diamine.

49. The method of claim 30, wherein said structural formula is structural formula VIII: 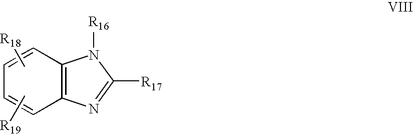
wherein, R16 is selected from the group consisting of a hydrogen and an amino group; R17 is selected from the group consisting of an amino group or a guanidino group, further wherein when said R16 is hydrogen, said R17 is a guanidino group or an amino group, and when said R16 is an amino group, said R17 is an amino group; R18 and R19 are independently selected from the group consisting of a hydrogen, a hydroxy, a lower alkyl group, a lower alkoxy group, and an aryl group; further wherein, said lower alkoxy group is selected from the group consisting of 1 to 6 carbon atoms, and said aryl group is selected from the group consisting of 6 to 10 carbon atoms.

50. The method of claim 49, wherein said compound is selected from the group consisting of 2-guanidinobenzimidazole, 1,2-diaminobenzimidazole, 1,2-diaminobenzimidazole hydrochloride, 5-bromo-2-guanidinobenzimidazole, 5-methoxy-2-guanidinobenzimidazole, 5-methylbenzimidazole- 1,2-diamine, 5-chlorobenzimidazole-1,2-diamine, and 2,5-diaminobenzimidazole.

51. The method of claim 30, wherein said structural formula is structural formula IX:
R₂₀—CH—(NHR₂₁)—CO₂H IX
wherein, R20 is selected from the group consisting of a hydrogen, a lower alkyl group, a lower alkylthiol group, a carboxy group, an aminocarboxy group and an amino group; R21 is selected from the group consisting of a hydrogen and an acyl group; further wherein said lower alkyl group is selected from the group consisting of 1 to 6 carbon atoms and said acyl group is selected from the group consisting of 2 to 10 carbon atoms.

52. The method of claim 51, wherein said compound is selected from the group is consisting of lysine, 2,3-diaminosuccinic acid, and cysteine.

53. The method of claim 30, wherein said compound is a compound comprising the formula of said structural formula X: 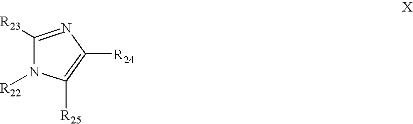
wherein R22 is selected from the group consisting of a hydrogen, an amino group, a mono-amino lower alkyl group, and a di-amino lower alkyl group; R23 is selected from the group consisting of a hydrogen, an amino group, a mono-amino lower alkyl group, and a di-amino lower alkyl group; R24 is selected from the group consisting of a hydrogen, a lower alkyl group, an aryl group and an acyl group; R25 is selected from the group consisting of a hydrogen, a lower alkyl group, an aryl group and an acyl group; further wherein, one of said R22 or R23 must be an amino group, or a mono- or di-amino lower alkyl group; said lower alkyl group is selected from the lower alkyl group consisting of 1 to 6 carbon atom; said mono- or di-amino alkyl groups are lower alkyl groups substituted by one or two amino groups; said aryl group is selected from the aryl group consisting of 6 to 10 carbon atoms; said acyl group is selected from the group consisting of a lower alkyl group, an aryl group, and a heteroaryl carboxylic acid containing 2 to 10 carbon atoms; and said lower alkoxy group is selected from the group consisting of 1 to 6 carbon atoms.

54. The method of claim 53, wherein said compound is selected from the group consisting of 1,2-diamino-4-phenyl[1H]imidazole, 1,2-diaminoimidazole, 1-(2,3-diaminopropyl)imidazole trihydrochloride, 4-(4-bromophenyl)imidazole-1,2-diamine, 4-(4-chlorophenyl)imidazole-1,2-diamine, 4-(4-hexylphenyl)imidazole- 1,2-diamine, 4-(4-methoxyphenyl)imidazole- 1,2-diamine, 4-phenyl-5-propylimidazole-1,2-diamine, 1,2-diamino-4-methylimidazole, 1,2-diamino-4,5-dimethylimidazole, and 1,2-diamino-4-methyl-5-acetylimidazole.

55. The method of claim 30, wherein said structural formula is structural formula XI: 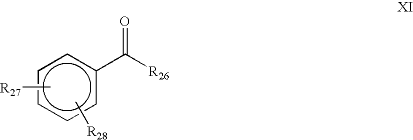
wherein R26 is selected from the group consisting of a hydroxy, a lower alkoxy group, an amino group, an amino lower alkoxy group, a mono-lower alkylamino lower alkoxy group, a di-lower alkylamino lower alkoxy group, a hydrazino group, and the formula NR29R30; R29 is selected from the group consisting of a hydrogen and a lower alkyl group; R30 is selected from the group consisting of an alkyl group of 1 to 20 carbon atoms, an aryl group, a hydroxy lower alkyl group, a carboxy lower alkyl group, a cyclo lower alkyl group and a heterocyclic group containing 4 to 7 ring members and 1 to 3 heteroatoms; further wherein, said R29, R30, and nitrogen form a structure selected from the group consisting of a morpholino, a piperidinyl, and a piperazinyl; R27 is selected from the group consisting of 0 to 3 amino groups, 0 to 3 nitro groups, 0 to 1 hydrazino group, a hydrazinosulfonyl group, a hydroxyethylamino group, and an amidino group; R28 is selected from the group consisting of a hydrogen, a one-fluoro, a two-fluoro, a hydroxy, a lower alkoxy, a carboxy, a lower alkylamino, a di-lower alkylamino and a hydroxy lower alkylamino group; further wherein, when said R26 is a hydroxy or a lower alkoxy, then said R27 is a non-hydrogen substituent; further wherein, when R26 is hydrazino, there must be at least two non-hydrogen substituents on said formula XI's phenyl ring; when said R28 is hydrogen, said R30 is selected from the group consisting of an alkyl group of 1 to 20 carbon atoms, an aryl group, a hydroxy lower alkyl group, a carboxy lower alkyl group, a cyclo lower alkyl group, a heterocyclic group containing 4 to 7 ring members and 1 to 3 heteroatoms, an aminoimino group, a guanidyl group, an aminoguanidinyl group, and a diaminoguanidyl group; said lower alkyl group is selected from the group consisting of 1 to 6 carbon atoms; and said cycloalkyl group is selected from the group consisting of 4 to 7 carbon atoms.

56. The method of claim 55, wherein said compound is selected from the group consisting of 4-(cyclohexylamino-carbonyl)-o-phenylene diamine hydrochloride, 3,4-diaminobenzhydrazide, 4-(n-butylamino-carbonyl)-o-phenylene-diamine dihydrochloride, 4-(ethylamino-carbonyl)-o-phenylene-diamine dihydrochloride, 4-carbamoyl-o-phenyiene diamine hydrochloride, 4-(morpholino-carbonyl)-o-phenylene-diamine hydrochloride, 4-[(4-morpholino)hydrazino-carbonyl]-o-phenylenediamine, 4-(1-piperidinylamino-carbonyl)-o-phenylenediamine dihydrochloride, 2,4-diamino-3-hydroxybenzoic acid, 4,5-diamino-2-hydroxybenzoic acid, 3,4-diaminobenzamide, 3,4-diaminobenzhydrazide, 3,4-diamino-N,N-bis (1-methylethyl)benzamide, 3,4-diamino-N,N-diethylbenzamide, 3,4-diamino-N,N-dipropylbenzamide, 3,4-diamino-N-(2-furanylmethyl)benzamide, 3,4-diamino-N-(2-methylpropyl)benzamide, 3,4-diamino-N-(5-methyl-2-thiazolyl)benzamide, 3,4-diamino-N-(6-methoxy-2-benzothiazolyl)benzamide, 3,4-diamino-N-(6-methoxy-8-quinolinyl)benzamide, 3,4-diamino-N-(6-methyl-2-pyridinyl)benzamide, 3,4-diamino-N-(1H-benzimidazol-2-yl)benzamide, 3,4-diamino-N-(2-pyridinyl)benzamide, 3,4-diamino-N-(2-thiazolyl)benzamide, 3,4-diamino-N -(4-pyridinyl)benzamide, 3,4-diamino-N-[9H-pyrido(3,4-b)indol-6-yl]benzamide, 3,4-diamino-N -butylbenzamide, 3,4-diamino-N-cyclohexylbenzamide, 3,4-diamino-N-cyclopentylbenzamide, 3,4-diamino-N-decylbenzamide, 3,4-diamino-N-dodecylbenzamide, 3,4-diamino-N -methylbenzamide, 3,4-diamino-N-octylbenzamide, 3,4-diamino-N-pentylbenzamide, 3,4-diamino-N-phenylbenzamide, 4-(diethylamino-carbonyl)-o-phenylene diamine, 4-(tert -butylamino-carbonyl)-o-phenylene diamine, 4-isobutylamino-carbonyl)-o-phenylene diamine, 4-(neopentylamino-carbonyl)-o-phenylene diamine, 4-(dipropylamino-carbonyl)-o-phenylene diamine, 4-(n-hexylamino-carbonyl)-o-phenylene diamine, 4-(n-decylamino-carbonyl)-o-phenylene diamine, 4-(n-dodecylamino-carbonyl)-o-phenylene diamine, 4-(1-hexadecylamino -carbonyl)-o-phenylene diamine, 4-(octadecylamino-carbonyl)-o-phenylene diamine,4-(hydroxylamino-carbonyl)-o-phenylene diamine, 4-(2-hydroxyethylamino-carbonyl)-o-phenylene, 4-[(2-hydroxyethylamino)ethylamino-carbonyl]-o-phenylene diamine, 4-[(2-hydroxyethyloxy)ethylamino-carbonyl]-o-phenylene diamine, 4-(6-hydroxyhexylamino -carbonyl)-o-phenylene diamine, 4-(3-ethoxypropylamino-carbonyl)-o-phenylene diamine, 4-(3-isopropoxypropylamino-carbonyl)-o-phenylene diamine, 4-(3-dimethylaminopropylamino -carbonyl)-o-phenylene diamine, 4-[4-(2-aminoethyl)morpholino-carbonyl]-o-phenylene diamine, 4-[4-(3-aminopropyl) morpholino-carbonyl]-o-phenylene diamine, 4-N-(3-aminopropyl)pyrrolidino-carbonyl]-o-phenylene diamine, 4-[3-(N-piperidino)propylamino -carbonyl]-o-phenylene diamine, 4-[3-(4-methylpiperazinyl)propylamino-carbonyl]-o-phenylene diamine, 4-(3-imidazoylpropylamino-carbonyl)-o-phenylene diamine, 4-(3-phenylpropylamino-carbonyl)-o-phenylenediamine, 4-[2-(N, N-diethylamino) ethylamino -carbonyl]-o-phenylene diamine, 4-(imidazolylamino-carbonyl)-o-phenylene diamine, 4-(pyrrolidinyl-carbonyl)-o-phenylene diamine, 4-(piperidino-carbonyl)-o-phenylene diamine, 4-(1-methylpiperazinyl-carbonyl)-o-phenylene diamine, 4-(2,6-dimethylmorpholino-carbonyl)-o-phenylenediamine, 4-(pyrrolidin-1 -ylamino-carbonyl)-o-phenylene diamine, 4-(homopiperidin-1-ylamino-carbonyl)-o-phenylene diamine, 4-(4-methylpiperazine- 1-ylamino-carbonyl)-o-phenylene diamine; 4-(1,2,4-triazol-1-ylamino-carbonyl)-o-phenylene diamine, 4-(guanidinyl -carbonyl)-o-phenylene diamine, 4-(guanidinylamino-carbonyl)-o-phenylene diamine, 4-aminoguanidinylamino-carbonyl)-o-phenylene diamine, 4-(diaminoguanidinylamino-carbonyl) -o-phenylene diamine, 3,4-aminosalicylic acid 4-guanidinobenzoic acid, 3,4-diaminobenzohydroxamic acid, 3,4,5-triaminobenzoic acid, 2,3-diamino-5-fluoro-benzoic acid, and 3,4-diaminobenzoic acid.

57. The method of claim 30, wherein said structural formula is structural formula XII: 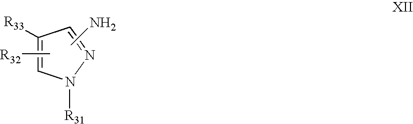
wherein R31 is selected from the group consisting of a hydrogen, a lower alkyl group and a hydroxy group; R32 is selected from the group consisting of a hydrogen, a hydroxy lower alkyl group, a lower alkoxy group, a lower alkyl group, and an aryl group; R33 is selected from the group consisting of a hydrogen and an amino group; said lower alkyl group is selected from the group consisting of 1 to 6 carbon atoms; said lower alkoxy group is selected from the group consisting of 1 to 6 carbon atoms; said hydroxy lower alkyl group is selected from the group consisting of primary, secondary and tertiary alcohol substituent patterns; said aryl group is selected from the group consisting of 6 to 10 carbon atoms; and a halo atom, wherein said halo atom is selected from the group consisting of a fluoro, a chloro, a bromo, and an iodo.

58. The method of claim 57, wherein said compound is selected from the group consisting of 3,4-diaminopyrazole, 3,4-diamino-5-hydroxypyrazole, 3,4-diamino-5-methylpyrazole, 3,4-diamino-5-methoxypyrazole, 3,4-diamino-5-phenylpyrazole, 1-methyl-3-hydroxy-4,5-diaminopyrazole, 1-(2-hydroxyethyl)-3-hydroxy-4,5-diaminopyrazole, 1-(2-hydroxyethyl)-3-phenyl-4,5-diaminopyrazole, 1-(2-hydroxyethyl)-3-methyl-4,5-diaminopyrazole, 1-(2-hydroxyethyl)-4,5-diaminopyrazole, 1-(2-hydroxypropyl)-3-hydroxy-4,5-diaminopyrazole, 3-amino-5-hydroxypyrazole, and 1-(2-hydroxy-2-methylpropyl)-3-hydroxy-4,5-diaminopyrazole.

59. The method of claim 30, wherein said structural formula is structural formula XIII: 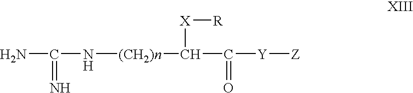
wherein n=1-6; X is selected from the group consisting of —NR1—, —S(O)—, —S(O)2—, and —O—, further wherein R1 is selected from the group consisting of H, linear chain alkyl group (C1-C6) and branched chain alkyl group (C1-C6); Y is selected from the group consisting of —N—, —NH—, and —O—; Z is selected from the group consisting of H, linear chain alkyl group (C1-C6), and branched chain alkyl group (C1-C6).

60. The method of claim 30, wherein said structural formula is structural formula XIV: 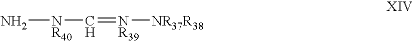
wherein R37 is selected from the group consisting of a lower alkyl group and a group of the formula NR41NR42; further wherein R41 and R42 together are selected from the group consisting of R41 is hydrogen and R42 is a lower alkyl group, R41 is hydrogen and R42 is a hydroxy (lower) alkyl group, and R41 and R42 together with said nitrogen atom form a heterocyclic group, further wherein said heterocyclic group contains 4 to 6 carbon atoms and 0 to 1 additional atoms selected from the group consisting of oxygen, nitrogen and sulfur; R38 is selected from the group consisting of a hydrogen and an amino group; R39 is selected from the group consisting of a hydrogen and an amino group; R40 is selected from the group consisting of a hydrogen and a lower alkyl group; further wherein at least one of said R38, R39, and R40 is other than hydrogen and one of said R37 and said R38 cannot be an amino group; said lower alkyl group is selected from the group consisting of 1 to 6 carbon atoms; said heterocyclic group formed by the NR41R42 group is a 4 to 7 membered ring containing 0 to 1 additional heteroatoms.

61. The method of claim 60, wherein said compound is selected from the group consisting of 2-(2-hydroxy-2-methylpropyl)hydrazinecarboximidic hydrazide, N-(4-morpholino)hydrazinecarboximidamide, 1-methyl-N-(4-morpholino)hydrazinecarboximidamide, 1-methyl-N-(4-piperidino)hydrazinecarboximidamide, 1 -(N-hexahydroazepino)hydrazinecarboximidamide, N,N-dimethylcarbonimidic dihydrazide, 1-methylcarbonimidic dihydrazide, 2-(2-hydroxy-2-methylpropyl) carbohydrazonic dihydrazide, and N-ethylcarbonimidic dihydrazide.

62. The method of claim 30, wherein said structural formula is structural formula V: 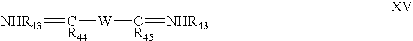
wherein R43 is selected from the group consisting of a pyridyl, a phenyl, and a carboxylic acid substituted phenyl group; wherein R46 is selected from the group consisting of a hydrogen, a lower alkyl group, and a water-solubilizing moiety; wherein W is selected from the group consisting of a carbon-carbon bond and an alkylene group of 1 to 3 carbon atoms; R44 is selected from the group consisting of a lower alkyl group, an aryl group, and a heteroaryl group; R45 is selected from the group consisting of a hydrogen, a lower alkyl group, an aryl group, and a heteroaryl group; said lower alkyl group is selected from the group consisting of 1 to 6 carbon atoms; said alkylene group is selected from the group consisting of a straight chain and a branched chain; said aryl group is selected from the group consisting of 6 to 10 carbon atoms; a halo atom is selected from the group consisting of a fluoro, a chloro, a bromo, and an iodo; said lower alkoxy group is selected from the group consisting of 1 to 6 carbon atoms; and said heteroaryl group is selected from the group consisting of 1 heteroatom and 2 heteroatoms.

63. The method of claim 62, wherein said compound is selected from the group consisting of methylglyoxal bis-(2-hydrazino-benzoic acid)hydrazone, methylglyoxal bis-(dimethyl-2-hydrazinobenzoate)hydrazone, methylglyoxal bis -(phenylhydrazine)hydrazone, methyl glyoxal bis-(dimethyl-2-hydrazinobenzoate)hydrazone, methylglyoxal bis-(4-hydrazinobenzoic acid)hydrazone, methylglyoxal bis-(dimethyl-4-hydrazinobenzoate)hydrazone, methylglyoxal bis-(2-pyridyl)hydrazone, methylglyoxal bis -(diethyleneglycol methylether-2-hydrazinobenzoate)hydrazone, methylglyoxal bis-[1-(2, 3-dihydroxypropane)-2-hydrazinebenzoatehydrazone, methyl glyoxal bis-[1-(2-hydroxyethane)-2-hydrazinobenzoate]hydrazone, methylglyoxal bis-[(1-hydroxymethyl-1-acetoxy))-2-hydrazino-2-benzoate]hydrazone, methylglyoxal bis-[(4-nitrophenyl)-2-hydrazinobenzoate]hydrazone, methylglyoxal bis-[(4-methylpyridyl)-2-hydrazinobenzoate]hydrazone, methylglyoxal bis -(triethylene glycol 2-hydrazinobenzoate)hydrazone, and methylglyoxal bis-(2-hydroxyethylphosphate-2-hydrazinebenzoate)hydrazone.

64. The method of claim 30, wherein said structural formula is structural formula XVI: 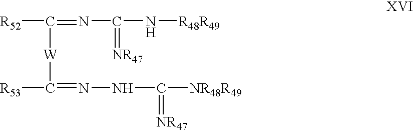
wherein R47 is selected from the group consisting of hydrogen and together with R48 an alkylene group of 2 to 3 carbon atoms; wherein said R48 is selected from the group consisting of hydrogen and alk-N-R5051, when said R47 is a hydrogen; further wherein, said alk is a straight or branched chain alkylene group of 1 to 8 carbon atoms, said R50 and R51 are independently each a lower alkyl group of 1 to 6 carbon atoms, or said R50 and said R51 together with said nitrogen atom form a group selected from the group consisting of a morpholino, a piperdinyl and a methylpiperazinyl; R49 is a hydrogen or said R49 is a hydroxyethyl when said R47 and said R48 are together an alkylene group of 2-3 carbon atoms; W is selected from the group consisting of a carbon-carbon bond, an alkylene group of 1 to 3 carbon atoms, a 1,2-, 1,3- or 1,4- phenylene group, a 2,3-naphthylene group, a 2,5-thiophenylene group, a 2,6-pyridylene group, an ethylene group, an ethenylene group, and a methylene group; R52 is selected from the group consisting of a lower alkyl group, an aryl group, and a heteroaryl group; R53 is selected from the group consisting of a hydrogen, a lower alkyl group, an aryl group, and a heteroaryl group; further wherein, when W is a carbon-carbon bond, R52 and R53 together can also be a 1,4-butylene group, or when W is a 1,2-, 1,3-, or 1,4-phenylene group, optionally substituted by one or two lower alkyl or amino groups, R52 and R53 are both hydrogen or a lower alkyl group; when W is an ethylene group, R52 and R53 together are an ethylene group; when W is a methylene group and R52 and R53 together are a group of the formula ═C (—CH3)—N—(H3C—) C═or—C—W—C—, then R52 and R53 together form a bicyclo—(3,3,1)-nonane or a bicyclo-3,3,1—octane group and R47 and R48 are together an alkylene group of 2-3 carbon atoms and R49 is hydrogen; said lower alkyl group is selected from the group consisting of 1 to 6 carbon atoms and said group may be optionally substituted by a halo hydroxy, an amino group or lower alkylamino group; said alkylene group is selected from the group consisting of straight and branched chain; said aryl group is selected from the group consisting of 6 to 10 carbon atoms; a halo atom, selected from the group consisting of a fluoro, a chloro, a bromo and an iodo; said lower alkoxy group is selecting from the group consisting of 1 to 6 carbon atoms, and said heteroaryl group is selected from the group consisting of 1 to 2 heteroatoms.

65. The method of claim 64, wherein said compound is selected from the group consisting of methyl glyoxal bis(guanylhydrazone), methyl glyoxal bis(2-hydrazino-2-imidazoline-hydrazone), terephthaldicarboxaldehyde bis(2-hydrazino-2-imidazoline hydrazone), terephaldicarboxaldehyde bis(guanylhydrazone), phenylglyoxal bis(2-hydrazino-2-imidazoline hydrazone), furylglyoxal bis(2-hydrazino-2-imidazoline hydrazone), methyl glyoxal bis (1-(2-hydroxyethyl)-2-hydrazino-2-imidazoline hydrazone), methyl glyoxal bis (1-(2-hydroxyethyl)-2-hydrazino-1,4,5,6-tetrahydropyrimidine hydrazone), phenyl glyoxal bis (guanylhydrazone), phenyl glyoxal bis (1-(2-hydroxyethyl)-2-hydrazino-2-imidazoline hydrazone), furyl glyoxal bis (1-(2-hydroxyethyl)-2-hydrazino-2-imidazoline hydrazone), phenyl glyoxal bis (1- (2-hydroxyethyl)-2-hydrazino-1,4,5,6-tetrahydropyrimidine hydrazone), furyl glyoxal bis (1-(2-hydroxyethyl)-2-hydrazino-1,4,5,6-tetrahydropyrimidine hydrazone), 2,3-butanedione bis (2-hydrazino-2- imidazoline hydrazone), 1,4-cyclohexanedione bis(2-hydrazino -2-imidazoline hydrazone), o-phthalic dicarboxaldehyde bis(2-hyd carboximidamide hydrazone), furylglyoxal bis(guanyl hydrazone)dihydrochloride dihydrate, 2,3-pentanedione bis(2-tetrahydropyrimidine) hydrazone dihydrobromide, 1,2-cyclohexanedione bis(2-tetrahydropyrimidine)hydrazone dihydrobromide, 2,3-hexanedione bis(2-tetrahydropyrimidine)hydrazone dihydrobromide, 1,3-diacetyl bis(2-tetrahydropyrimidine) hydrazone dihydrobromide, 2,3-butanedione bis(2-tetrahydropyrimidine)hydrazone dihydrobromide, 2,6-diacetylpyridine-bis-(2-hydrazino-2-imidazoline hydrazone)dihydrobromide; 2,6-diacetylpyridine-bis-(guanyl hydrazone)dihydrochloride, 2,6-pyridine dicarboxaldehyde-bis-(2-hydrazino-2-imidazoline hydrazone)dihydrobromide trihydrate), 2,6-pyridine dicarboxaldehyde-bis (guanyl hydrazone)dihydrochloride,; 1,4-diacetyl benzene-bis-(2-hydrazino-2-imidazoline hydrazone) dihydrobromide dihydrate, 1,3-diacetyl benzene-bis-(2-hydrazino-2-imidazoline) hydrazone dihydrobromide, 1,3-diacetyl benzene-bis (guanyl)-hydrazone dihydrochloride, isophthalaldehyde-bis-(2-hydrazino-2-imidazoline) hydrazone dihydrobromide, isophthalaldehyde-bis-(guanyl)hydrazone dihydrochloride, 2,6-diacetylaniline bis -(guanyl)hydrazone dihydrochloride, 2,6-diacetyl aniline bis-(2-hydrazino-2-imidazoline)hydrazone dihydrobromide, 2,5-diacetylthiophene bis(guanyl)hydrazone dihydrochloride, 2,5-diacetylthiophene bis-(2-hydrazino-2-imidazoline)hydrazone dihydrobromide, 1,4-cyclohexanedione bis(2-tetrahydropyrimidine)hydrazone dihydrobromide, 3,4-hexanedione bis(2-tetrahydropyrimidine)hydrazone dihydrobromide, methylglyoxal-bis-(4-amino-3-hydrazino-1,2,4-triazole)hydrazone dihydrochloride, methylglyoxal-bis-(4-amino-3-hydrazino-5-methyl-1,2,4-triazole)hydrazone dihydrochloride, 2,3-pentanedione-bis- (2-hydrazino-3-imidazoline)hydrazone dihydrobromide, 2,3-hexanedione-bis-(2-hydrazino-2-imidazoline)hydrazone dihydrobromide, 3-ethyl-2,4-pentane dione-bis- (2-hydrazino-2-imidazoline)hydrazone dihydrobromide, methylglyoxal-bis-(4-amino-3-hydrazino-5-ethyl-1,2,4-triazole)hydrazone dihydrochloride, methylglyoxal-bis-(4-amino-3-hydrazino-5-isopropyl-1,2,4triazole)hydrazone dihydrochloride, methyl glyoxal-bis-(4-amino-3-hydrazino-5-cyclopropyl-1,2,4-triazole)hydrazone dihydrochlorimethylglyoxal-bis-(4-amino-3-hydrazino-5-cyclobutyl-1,2,4-triazole) hydrazone dihydrochloride, 1,3-cyclohexanedione-bis-(2-hydrazino-2-imidazoline) hydrazone dihydrobromide, 6-dimethyl pyridine bis(guanyl)hydrazone dihydrochloride, 3,5-diacetyl- 1,4-dihydro-2,6-dimethylpyridine bis-(2-hydrazino-2-imidazoline hydrazone dihydrobromide, bicyclo-(3,3,1)nonane-3,7-dione bis- (2-hydrazino-2-imidazoline)hydrazone dihydrobromide, and cis-bicyclo-(3,3,1)octane-3,7-dione bis-(2-hydrazino-2-imidazoline)hydrazone dihydrobromide.

66. The method of claim 30, wherein said structural formula is structural formula XVII: 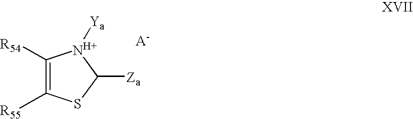
wherein R54 is selected from the group consisting of a hydrogen, a hydroxy (lower) alkyl group, a lower acyloxy (lower) alkyl group, and a lower alkyl group; R55 is selected from the group consisting of a hydrogen, a hydroxy (lower) alkyl group, a lower acyloxy (lower) alkyl group, and a lower alkyl group; further wherein R54 and R55 together with their ring carbons may be an aromatic fused ring; Za is hydrogen or an amino group; Ya is selected from the group consisting of a hydrogen, a group of the formula —CH2C(═O)—R56, and a group of the formula —CHR′, further wherein, when said Ya is a group of said formula —CH2C(=O)—R56, said R is selected from the group consisting of a lower alkyl group, an alkoxy group, a hydroxy, an amino group, and an aryl group; wherein when said Ya is a group of said formula —CHR′, said R′ is selected from the group consisting of a hydrogen, a lower alkyl group, a lower alkynyl group, and an aryl group; wherein A is selected from the group consisting of a halide, a tosylate, a methanesulfonate, and a mesitylenesulfonate ion; said lower alkyl group is selected from the group consisting of 1-6 carbon atoms; said lower alkynyl group is selected from the group consisting of 2 to 6 carbon atoms; said lower alkoxy group is selected from the group consisting of 1 to 6 carbon atoms; said lower acyloxy (lower) alkyl group contains an acyloxy portion and a lower alkyl portion, further wherein said acyloxy portion is selected from the group consisting of 2 to 6 carbon atoms and said lower alkyl portion is selected from the group consisting of 1 to 6 carbon atoms; said aryl group is selected from the group consisting of 6 to 10 carbon atoms; and a halo atom of formula XVII is selected from the group consisting of a fluoro, a chloro, a bromo, and an iodo.

67. The method of claim 66, wherein said compound is selected from the group consisting of 3-aminothiazolium mesitylenesulfonate, 3-amino-4,5-dimethylaminothiazolium mesitylenesulfonate, 2,3-diaminothiazolinium mesitylenesulfonate, 3-(2-methoxy-2-oxoethyl)-thiazolium bromide, 3-(2-methoxy-2-oxoethyl)-4,5-dimethylthiazolium bromide, 3-(2-methoxy-2-oxoethyl)-4-methylthiazolium bromide, 3-(2-phenyl-2-oxoethyl)-4-methylthizolium bromide, 3-(2-phenyl-2-oxoethyl)-4,5-dimethylthiazolium bromide, 3-amino-4-methylthiazolium mesitylenesulfonate, 3-(2-methoxy-2-oxoethyl)-5-methylthiazolium bromide, 3-(3-(2-phenyl-2-oxoethyl)-5-methylthiazolium bromide, 3-[2-(4′-bromophenyl)-2-oxoethyl]thiazolium bromide, 3-[2-(4′-bromophenyl)-2-oxoethyl]-4-methylthiazolium bromide, 3-[2-(4′-bromophenyl)-2-oxoethyl]-5-methylthiazolium bromide, 3-[2-(4′bromophenyl)-2-oxoethyl]-4,5-dimethylthiazolium bromide, 3-(2-methoxy-2-oxoethyl)-4-methyl-5-(2-hydroxyethyl) thiazolium bromide, 3-(2-phenyl-2-oxoethyl)-4-methyl-5-(2-hydroxyethyl) thiazolium bromide, 3-[2-(4′-bromophenyl)-2-oxoethyl]-4-methyl-5-(2-hydroxyethyl) thiazolium bromide, 3,4-dimethyl-5-(2-hydroxyethyl) thiazolium iodide, 3-ethyl-5-(2-hydroxyethyl)-4-methylthiazolium bromide, 3-benzyl-5-(2-hydroxyethyl)-4-methylthiazolium chloride, 3-(2-methoxy-2-oxoethyl)benzothiazolium bromide, 3-(2-phenyl-2-oxoethyl)benzothiazolium bromide, 3-[2-(4′bromophenyl)-2-oxoethyl] benzothiazolium bromide, 3-(carboxymethyl) benzothiazolium bromide, 2,3-(diamino) benzothiazolium mesitylenesulfonate, 3-(2-amino-2-oxoethyl) thiazolium bromide, 3-(2-amino-2-oxoethyl)-4-methylthiazolium bromide, 3-(2-amino-2-oxoethyl)-5-methylthiazolium bromide, 3-(2-amino-2-oxoethyl) 4,5-dimethylthiazolium bromide, 3-(2-amino-2-oxoethyl)benzothiazolium bromide, 3-(2-amino-2- oxoethyl) 4-methyl-5-(2-hydroxyethyl)thiazolium bromide, 3-amino-5-(2-hydroxyethyl)-4-methylthiazolium mesitylenesulfonate, 3-(2-methyl-2-oxoethyl)thiazolium chloride, 3-amino-4-methyl-5-(2-acetoxyethyl)thiazolium mesitylenesulfonate, 3-(2-phenyl-2-oxoethyl)thiazolium bromide, 3-(2-methoxy-2-oxoethyl)-4-methyl-5-(2-acetoxyethyl) thiazoliumbromide, 3-(2-amino-2-oxoethyl) -4-methyl-5- (2-acetoxyethyl)thiazolium bromide, 2-amino-3- (2-methoxy-2-oxoethyl) thiazolium bromide, 2-amino-3 -(2 -methoxy-2-oxoethyl) benzothiazolium bromide, 2-amino-3 -(2-amino-2-oxoethyl)thiazolium bromide, 2-amino-3-(2-amino-2-oxoethyl)benzothiazolium bromide, 3-[2-(4′-methoxyphenyl)-2-oxoethyl]-thiazolinium bromide, 3-[2-(2′,4′-dimethoxyphenyl)-2-oxoethyl]-thiazolinium bromide, 3-[2-(4′-fluorophenyl)-2-oxoethyl]-thiazolinium bromide, 3-[2-(2′,4′-difluorophenyl)-2-oxoethyl]-thiazolinium bromide, 3-[2-(4′-diethylaminophenyl)-2-oxoethyl]-thiazolinium bromide, 3-propargyl-thiazolinium bromide, 3-propargyl-4-methylthiazolinium bromide, 3-propargyl-5-methylthiazolinium bromide, 3-propargyl-4,5-dimethylthiazolinium bromide, and 3-propargyl-4-methyl-5-(2-hydroxyethyl)-thiazolinium bromide.

68. The method of claim 30, wherein said structural formula is structural formula XVIII: 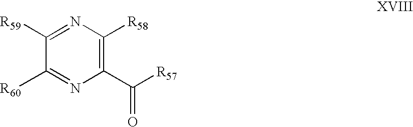
wherein, R57 is selected from the group consisting of a hydroxy, a NHCONCR61R62, and a N═C(NR61R62)2; R61 and R62 are each independently selected from the group consisting of a hydrogen, a 1 to 10 carbon atom straight chain alkyl, a 1 to 10 carbon atom branched chain alkyl, an aryl 1 to 4 carbon atom alkyl, a mono-substituted aryl 1 to 4 carbon alkyl, and a di-substituted aryl 1 to 4 carbon atom alkyl, wherein said substituents are selected from the group consisting of a fluoro, a chloro, a bromo, an iodo, a 1 to 10 carbon atom alkyl straight chain, and a 1 to 10 carbon atom alkyl branched chain; wherein R58 is selected from the group consisting of a hydrogen, an amino, a mono-substituted amino and a di-substituted amino, and R59 is selected from the group consisting of a hydrogen, an amino, a mono-substituted amino and a di-substituted amino; further wherein, when R58 and R59 are not both amino or substituted amino, the substituents are selected from the group consisting of a 1 to 10 carbon atom straight chain alkyl, a 1 to 10 carbon atom branched chain alkyl, and a 3 to 8 carbon atom cycloalkyl; and wherein R60 is selected from the group consisting of a hydrogen, a trifluoromethyl, a fluoro, a chloro, a bromo, and an iodo.

69. A method of treating a mammal having a disease selected from the group consisting of scleroderma, keloids, and scarring, wherein said mammal is in need of said treatment, said method comprising administering to said mammal an effective amount of a composition comprising at least one compound capable of disrupting a crosslinkage between crosslinked proteins.

70. The method of claim 69, wherein said compound is selected from the group consisting of compounds of the formula XXV: 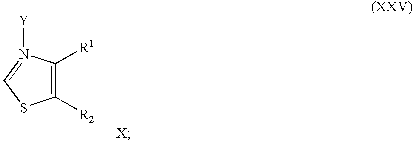
wherein R.sup.1 and R.sup.2 are independently selected from the group consisting of hydrogen and an alkyl group, which can be substituted by a hydroxy group;
Y is a group of the formula —CH.sub.2 C(═O)R wherein R is a heterocyclic group other than alkylenedioxyaryl containing 4-10 ring members and 1-3 heteroatoms selected from the group consisting of oxygen, nitrogen and sulfur, the heterocyclic group can be substituted by one or more substituents selected from the group consisting of alkyl, oxo, alkoxycarbonylalkyl, aryl, and aralkyl groups; and said one or more substituents can be substituted by one or more alkyl or alkoxy groups; or group of the formula —CH.sub.2 C(.dbd.O)—NHR′ wherein R′ is a heterocyclic group other than alkylenedioxyaryl containing 4-10 ring members and 1-3 heteroatoms selected from the group consisting of oxygen, nitrogen, and sulfur, the heterocyclic group can be substituted by one or more alkoxycarbonylalkyl groups; and
X is a pharmaceutically acceptable ion; and a carrier therefor.

71. A method of treating a mammal having a disease selected from the group consisting of scleroderma, keloids, and scarring, wherein said mammal is in need of said treatment, said method comprising administering to said mammal an effective amount of a composition comprising at least one compound capable of preventing protein crosslinkage.

72. A method of treating a mammal having a disease selected from the group consisting of scleroderma, keloids, and scarring, wherein said mammal is in need of said treatment, said method comprising administering to said mammal an effective amount of a composition comprising:
a) at least one compound capable of preventing protein crosslinkage; and
b) at least one compound capable of disrupting a crosslinkage between crosslinked proteins.

73. A method of preventing the crosslinking of collagen in a patient in need thereof, said method comprising administering to said patient a composition comprising a compound that inactivates 3DG.

74. The method of claim 73, wherein said compound inhibits the formation of 3DG.

75. The method of claim 73, wherein said compound is selected from the group consisting of compounds having structural formula I: 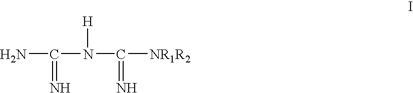
wherein R1 and R2 are independently selected from the group consisting of a hydrogen, a lower alkyl, a lower alkoxy and an aryl group; or wherein said R1 and said R2 together with a nitrogen atom form a heterocyclic ring containing from 1 to 2 heteroatoms and 2 to 6 carbon atoms, the second of said heteroatoms comprising nitrogen, oxygen, or sulfur; further wherein said lower alkyl group is selected from the group consisting of 1 to 6 carbon atoms; wherein said lower alkoxy group is selected from the group consisting of 1 to 6 carbon atoms; and wherein said aryl group comprises substituted and unsubstituted phenyl and pyridyl groups.

76. The method of claim 74, wherein said compound is selected from the group consisting of meglumine, sorbitollysine, mannitollysine and galactitollysine.

77. The method of claim 73, wherein said patient has at least one disease selected from the group consisting of scleroderma, keloids and scarring.

78. A method of inhibiting fructosamine kinase in a mammal, said method comprising administering to said mammal a composition comprising a copper-containing compound.

79. The method of claim 78, wherein said copper-containing compound is selected from the group consisting of a copper-salicylic acid conjugate, a copper-peptide conjugate, a copper-amino acid conjugate, and a copper salt.

80. The method of claim 79, wherein said copper-containing compound is selected from the group consisting of a copper-lysine conjugate and a copper-arginine conjugate.

81. The method of claim 78, wherein said mammal has a disease associated with at least one diabetic complication.

82. A method of claim 81, wherein said diabetic complication is selected from the group consisting of retinopathy, neuropathy, cardiovascular disease, dementia, and nephropathy.

83. A method of increasing the production of collagen in a mammal by administering to said mammal a composition that inhibits the Amadorase pathway, wherein said composition comprises a copper-containing compound, thereby increasing the production of collagen in said mammal.

84. The method of claim 83, wherein said copper-containing compound inhibits fructoseamine kinase.

85. The method of claim 83, wherein said collagen is Type I collagen.

86. The method of claim 83, wherein said collagen is Type III collagen.

87. The method of claim 67, wherein said collagen comprises Type I and Type III collagens.

88. A method of increasing the level of mRNA for collagen in a mammal, said method comprising administering to said mammal a composition that inhibits the Amadorase pathway, said composition comprising a copper-containing compound, thereby increasing the level of mRNA collagen in said mammal.

89. A method of decreasing desmosine levels in a mammal, said method comprising administering to said mammal a composition comprising an inhibitor of the Amadorase pathway, wherein said inhibitor is a copper-containing compound.

90. A method of stabilizing desmosine levels in a mammal, said method comprising administering to said mammal a composition comprising an inhibitor of the Amadorase pathway, wherein said inhibitor is a copper-containing compound.

91. A method of decreasing the level of mRNA for collagen in a mammal by increasing the flux through the Amadori pathway in said mammal, said method comprising administering to said mammal a composition comprising at least one copper chelator.

92. The method of claim 91, wherein said compound is selected from the group consisting of triethylenetetramine dihydrochloride (triene), penicillamine, sar, diamsar, ethylenediamine tetraacetic acid, o-phenanthroline, and histidine.


SEQUENCE LISTING (2 sequences)

SEQ ID NO: 1 - DNA, 988 bp, Homo sapiens
cgtcaagcttggcacgaggccatggagcagctgctgcgcgccgagctgcgcaccgcgaccctgcgggccttcggcggccccggcgccggctgcatcagcgagggccgagcctacgacacggacgcaggcccagtgttcgtcaaagtcaaccgcaggacgcaggcccggcagatgtttgagggggaggtggccagcctggaggccctccggagcacgggcctggtgcgggtgccgaggcccatgaaggtcatcgacctgccgggaggtggggccgcctttgtgatggagcatttgaagatgaagagcttgagcagtcaagcatcaaaacttggagagcagatggcagatttgcatctttacaaccagaagctcagggagaagttgaaggaggaggagaacacagtgggccgaagaggtgagggtgctgagcctcagtatgtggacaagttcggcttccacacggtgacgtgctgcggcttcatcccgcaggtgaatgagtggcaggatgactggccgacctttttcgcccggcaccggctccaggcgcagctggacctcattgagaaggactatgctgaccgagaggcacgagaactctggtcccggctacaggtgaagatcccggatctgttttgtggcctagagattgtccccgcgttgctccacggggatctctggtcgggaaacgtggctgaggacgacgtggggcccattatttacgacccggcttccttctatggccattccgagtttgaactggcaatcgccttgatgtttggggggttccccagatccttcttcaccgcctaccaccggaagatccccaaggctccgggcttcgaccagcggctgctgctctaccagctgtttaactacctgaaccactggaaccacttcgggcgggagtacaggagcccttccttgggcaccatgcgaaggctgctcaagtagcggcccctgccctcccttcccctgtccccgtccccgt

SEQ ID NO: 2 - protein, 309 aa, Homo sapiens
MEQLLRAELRTATLRAFGGPGAGCISEGRAYDTDAGPVFVKVNRRTQARQMFEGEVASLEALRSTGLVRVPRPMKVIDLPGGGAAFVMEHLKMKSLSSQASKLGEQMADLHLYNQKLREKLKEEENTVGRRGEGAEPQYVDKFGFHTVTCCGFIPQVNEWQDDWPTFFARHRLQAQLDLIEKDYADREARELWSRLQVKIPDLFCGLEIVPALLHGDLWSGNVAEDDVGPIIYDPASFYGHSEFELAIALMFGGFPRSFFTAYHRKIPKAPGFDQRLLLYQLFNYLNHWNHFGREYRSPSLGTMRRLLK